(19) United States
(12) Patent Application Publication
(10) Pub. No.: US 2013/0005596 A1
(43) Pub. Date: Jan. 3, 2013

(54) NOVEL GENOMIC BIOMARKERS FOR IRRITABLE BOWEL SYNDROME DIAGNOSIS

(75) Inventors: Hua Gong, San Diego, CA (US); Sharat Singh, Rancho Santa Fe, CA (US); Nicholas Hoe, San Diego, CA (US)

(73) Assignee: Nestec S.A., Vevey (CH)

(21) Appl. No.: 13/479,127

(22) Filed: May 23, 2012

Related U.S. Application Data
(63) Continuation of application No. PCT/US10/58099, filed on Nov. 24, 2010.
(60) Provisional application No. 61/264,634, filed on Nov. 25, 2009.

(51) Int. Cl. C40B 30/04; G01N 27/62; C12Q 1/68

(52) U.S. Cl. 506/9; 435/6.11; 435/6.12

(57) ABSTRACT

The invention provides novel biomarkers, kits, and methods of diagnosing, prognosing, and subtyping IBS. In one aspect, the invention provides novel genomic biomarkers for diagnosing, classifying, providing a prognosis for, and assigning therapy for IBS in a subject in need thereof. In another aspect, the present invention provides novel algorithms for the diagnosis and prognosis of IBS.

### CROSS-REFERENCE TO RELATED APPLICATIONS

This application is a continuation of PCT/US2010/058099, filed Nov. 24, 2010, which application claims priority to U.S. Application No. 61/264,634, filed Nov. 25, 2009, the teachings of which are incorporated herein by reference in their entirety for all purposes.

### BACKGROUND OF THE INVENTION

Irritable bowel syndrome (IBS) is the most common of all gastrointestinal disorders, affecting 10-20% of the general population and accounting for more than 50% of all patients with digestive complaints. However, studies suggest that only about 10% to 50% of those afflicted with IBS actually seek medical attention. Patients with IBS present with disparate symptoms such as, for example, abdominal pain predominantly related to defecation, diarrhea, constipation or alternating diarrhea and constipation, abdominal distention, gas, and excessive mucus in the stool. More than 40% of IBS patients have symptoms so severe that they have to take time off from work, curtail their social life, avoid sexual intercourse, cancel appointments, stop traveling, take medication, and even stay confined to their house for fear of embarrassment. The estimated health care cost of IBS in the United States is $8 billion per year (Talley et al., Gastroenterol., 109:1736-1741 (1995)).

The precise pathophysiology of IBS is not well understood. Nevertheless, there is a heightened sensitivity to visceral pain perception, known as peripheral sensitization. This sensitization involves a reduction in the threshold and an increase in the gain of the transduction processes of primary afferent neurons, attributable to a variety of mediators including monoamines (e.g., catecholamines and indoleamines), substance P, and a variety of cytokines and prostanoids such as E-type prostaglandins (see, e.g., Mayer et al., Gastroenterol., 107:271-293 (1994)). Also implicated in the etiopathology of IBS is intestinal motor dysfunction, which leads to abnormal handling of intraluminal contents and/or gas (see, e.g., Kellow et al., Gastroenterol., 92:1885-1893 (1987); Levitt et al., Ann. Int. Med., 124:422-424 (1996)). Psychological factors may also contribute to IBS symptoms appearing in conjunction with, if not triggered by, disturbances including depression and anxiety (see, e.g., Drossman et al., Gastroenterol. Int., 8:47-90 (1995)).

The causes of IBS are not well understood. The walls of the intestines are lined with layers of muscle that contract and relax as they move food from the stomach through the intestinal tract to the rectum. Normally, these muscles contract and relax in a coordinated rhythm. In IBS patients, these contractions are typically stronger and last longer than normal. As a result, food is forced through the intestines more quickly in some cases causing gas, bloating, and diarrhea. In other cases, the opposite occurs: food passage slows and stools become hard and dry causing constipation.

The precise pathophysiology of IBS remains to be elucidated. While gut dysmotility and altered visceral perception are considered important contributors to symptom pathogenesis (Quigley, Scand. J. Gastroenterol., 38 (Suppl. 237): 1-8 (2003); Mayer et al., Gastroenterol., 122:2032-2048 (2002)), this condition is now generally viewed as a disorder of the brain-gut axis. Recently, roles for enteric infection and intestinal inflammation have also been proposed. Studies have documented the onset of IBS following bacteriologically confirmed gastroenteritis, while others have provided evidence of low-grade mucosal inflammation (Spiller et al., Gut, 47:804-811 (2000); Dunlop et al., Gastroenterol., 125:1651-1659 (2003); Cumberland et al., Epidemiol. Infect., 130:453-460 (2003)) and immune activation (Gwee et al., Gut, 52:523-526 (2003); Pimentel et al., Am. J. Gastroenterol., 95:3503-3506 (2000)) in IBS. The enteric flora has also been implicated, and a recent study demonstrated the efficacy of the probiotic organism Bifidobacterium in treating the disorder through modulation of immune activity (O'Mahony et al., Gastroenterol., 128:541-551 (2005)).

The hypothalamic-pituitary-adrenal axis (HPA) is the core endocrine stress system in humans (De Wied et al., Front. Neuroendocrinol., 14:251-302 (1993)) and provides an important link between the brain and the gut immune system. Activation of the axis takes place in response to both physical and psychological stressors (Dinan, Br. J. Psychiatry, 164 :365-371 (1994)), both of which have been implicated in the pathophysiology of IBS (Cumberland et al., Epidemiol. Infect., 130:453-460 (2003)). Patients with IBS have been reported as having an increased rate of sexual and physical abuse in childhood together with higher rates of stressful life events in adulthood (Gaynes et al., Baillieres Clin. Gastroenterol., 13:437-452 (1999)). Such psychosocial trauma or poor cognitive coping strategy profoundly affects symptom severity, daily functioning, and health outcome.

Although the etiology of IBS is not fully characterized, the medical community has developed a consensus definition and criteria, known as the Rome II criteria, to aid in the diagnosis of IBS based upon patient history. The Rome II criteria requires three months of continuous or recurrent abdominal pain or discomfort over a one-year period that is relieved by defecation and/or associated with a change in stool frequency or consistency as well as two or more of the following: altered stool frequency, altered stool form, altered stool passage, passage of mucus, or bloating and abdominal distention. The absence of any structural or biochemical disorders that could be causing the symptoms is also a necessary condition. As a result, the Rome II criteria can be used only when there is a substantial patient history and is reliable only when there is no abnormal intestinal anatomy or metabolic process that would otherwise explain the symptoms. Similarly, the Rome III criteria recently developed by the medical community can be used only when there is presentation of a specific set of symptoms, a detailed patient history, and a physical examination.

It is well documented that diagnosing a patient as having IBS can be challenging due to the similarity in symptoms between IBS and other diseases or disorders. In fact, because the symptoms of IBS are similar or identical to the symptoms of so many other intestinal illnesses, it can take years before a correct diagnosis is made. For example, patients who have inflammatory bowel disease (IBD), but who exhibit mild signs and symptoms such as bloating, diarrhea, constipation, and abdominal pain, may be difficult to distinguish from patients with IBS. As a result, the similarity in symptoms between IBS and IBD renders rapid and accurate diagnosis difficult. The difficulty in differentially diagnosing IBS and IBD hampers early and effective treatment of these diseases. Unfortunately, rapid and accurate diagnostic methods for definitively distinguishing IBS from other intestinal diseases or disorders presenting with similar symptoms are currently not available. The present invention satisfies this need and provides related advantages as well.

### BRIEF SUMMARY OF THE INVENTION

The present invention provides methods, systems, and code for accurately classifying whether a sample from an individual is associated with Irritable Bowel Syndrome (IBS) or a subtype thereof. As a non-limiting example, the present invention is useful for classifying a sample from an individual as an IBS sample using a statistical algorithm and/or empirical data. The present invention is also useful for ruling out one or more diseases or disorders that present with IBS-like symptoms and ruling in IBS using a combination of statistical algorithms and/or empirical data. Thus, the present invention provides an accurate diagnostic prediction of IBS, classification of an IBS subtype, and prognostic information useful for guiding treatment decisions.

In one aspect, the present invention provides a method for diagnosing Irritable Bowel Syndrome (IBS) in a subject in need thereof, the method comprising: (a) isolating and/or amplifying RNA from a biological sample taken from the subject; (b) contacting the isolated and/or amplified RNA with a detection reagent under conditions suitable to transform the detection reagent into a complex comprising the detection reagent and an IBS RNA biomarker; (c) detecting the level of the complex; and (d) determining if the level of the complex more closely resembles a first reference level associated with IBS or a second reference level associated with an absence of IBS, thereby diagnosing IBS or a subtype thereof in the subject, wherein the biomarker is an RNA from a gene selected from the group consisting of those found in Table 4 such as CCDC147. In another embodiment, the gene is selected from the group consisting of those found in Table 6. In a more preferred embodiment, the gene is selected from the group consisting of those found in Table 7.

In another aspect, the present invention provides a method for monitoring the progression or regression of Irritable Bowel Syndrome (IBS) in a subject, said method comprising: (a) determining a first biomarker profile from a first biological sample taken from the subject at a first point in time; (b) determining a second biomarker profile from a second biological sample taken from the subject at a second point in time; and (c) comparing said first and said second biomarker profiles to (i) determine which biomarker profile most resembles or least resembles a first reference profile associated with IBS, (ii) determine which biomarker profile least resembles or most resembles a second reference profile associated with the absence of IBS, or (iii) determining at least two of the foregoing resemblances, wherein the biomarker profiles comprise information about the expression of at least 2 biomarkers found in Table 4, thereby monitoring progression or regression of IBS in said subject. In another embodiment, biomarker profiles comprise information about the expression of at least 2 biomarkers found in Table 6. In a more preferred embodiment, the biomarker profiles comprise information about the expression of at least 2 biomarkers found in Table 7.

In yet another aspect, the present invention provides a method for assigning therapy for IBS to a subject in need thereof, the method comprising: (a) isolating and/or amplifying RNA from a biological sample taken from the subject; (b) contacting the isolated and/or amplified RNA with a detection reagent under conditions suitable to transform the detection reagent into a complex comprising the detection reagent and an IBS RNA biomarker; (c) detecting the level of the complex; (d) determining if the level of the complex more closely resembles a first reference level associated with IBS or a second reference level associated with an absence of IBS; and (e) assigning therapy for IBS if said level more closely resembles said first reference level associated with IBS, wherein the IBS RNA biomarker is selected from the group consisting of those found in Table 4. In another embodiment, the gene is selected from the group consisting of those found in Table 6. In a more preferred embodiment, the gene is selected from the group consisting of those found in Table 7.

In certain embodiments, the methods of the invention further comprise classifying a sample as an IBS-constipation (IBS-C), IBS-diarrhea (IBS-D), IBS-mixed (IBS-M), IBS-alternating (IBS-A), or post-infectious IBS (IBS-PI) sample. In other embodiments, the methods further comprise classifying a non-IBS sample as a normal, inflammatory bowel disease (IBD), or non-IBD sample.

In certain embodiments, the methods for diagnosing IBS, monitoring the progression or regression of IBS and/or assigning therapy for IBS comprise the detection of at least 2, 3, 4, 5, or more of the biomarkers found in Table 2, Table 4, Table 6, and Table 7. In other embodiments, the methods further comprises the detection of a biomarker selected from the group consisting of a cytokine, a growth factor, an anti-neutrophil antibody, an anti-Saccharomyces cerevisiae antibody, an antimicrobial antibody, an anti-tissue transglutaminase (tTG) antibody, a lipocalin, a matrix metalloproteinase (MMP), a complex of lipocalin and MMP, a tissue inhibitor of metalloproteinases (TIMPs), a globulin (e.g., an alpha-globulin), an actin-severing protein, an S100 protein, a fibrinopeptide, calcitonin gene-related peptide (CGRP), a tachykinin, ghrelin, neurotensin, corticotropin-releasing hormone (CRH), elastase, C-reactive protein (CRP), lactoferrin, an anti-lactoferrin antibody, calprotectin, hemoglobin, NOD2/CARD15, serotonin reuptake transporter (SERT), tryptophan hydroxylase-1, 5-hydroxytryptamine (5-HT), lactulose, and a combination thereof.

In certain other embodiments, the methods of the present invention further comprise determining a symptom profile, wherein said symptom profile is determined by identifying the presence or severity of at least one symptom in said individual; and classifying said sample as an IBS sample or non-IBS sample using an algorithm based upon said diagnostic marker profile and said symptom profile.

These and other objects, aspects and embodiments will become more apparent with the detailed description and figures that follow.

### BRIEF DESCRIPTION OF THE DRAWINGS

FIG. 1 illustrates a Box-and-Whisker plot of the gene expression data for the eight training samples after processing via the RMA algorithm. Samples HG1 and 2 correspond to IBS-C samples, HG3, 4, and 5 correspond to IBS-D, and HG6, 7, and 8 correspond to healthy control samples.

FIG. 2 illustrates gene plots of the top 5 differentially expressed genes based on ANOVA analysis.

FIG. 3 illustrates the clustering results of unsupervised hierarchical clustering analysis performed using all probe sets on the arrays.

FIG. 4 illustrates a heat map of the clustering results of unsupervised hierarchical clustering analysis performed using all unmasked probe sets on the arrays. Global expression analysis of transcipts from IBS patients and healthy volunteers. Total RNA obtained from 3 IBS-D, 2 IBS-C and 3 healthy volunteers were analyzed on Affymetrix array containing more than 35,000 human genes. An unsupervisored hierarchical cluster analysis of 3 normal and 5 IBS samples. Red indicates genes that are elevated relative to the average expression values across all experiments. Green indicates genes that are decreased relative to the average expression value.

FIG. 5 illustrates a multidimensional scaling plot to visualize the separation among samples based on the gene expression profiles of all unmasked probe sets.

FIG. 6 illustrates the principal component analysis results. The left plot (A) shows how many percent of total variation can be explained by the top principal components. The right plot (B) shows the separation of the samples by the top 2 principal components.

FIGS. 7A-C illustrate volcano plots of the comparison between each pair of groups, specifically, between (A) IBS-C and IBS-D groups, (B) IBS-C and control groups, and (C) IBS-D and control groups.

FIG. 8 illustrates the results of qRT-PCR validation of the differential gene expression of the FOXD3, PI4K2A, ACSS2, ASIP, and OR2L8 genes in samples from IBS-M, IBS-C, IBS-D, and control (HV) subjects.

FIGS. 9A-C illustrate the results of qRT-PCR validation of the differential gene expression of the selected candidate biomarkers in samples from IBS-M, IBS-C, IBS-D, and control (HV) subjects.

FIGS. 10A-C illustrate the results of real time quantitative PCR validation of the expression of 36 selected differently expressed genes (DEGs) in samples from IBS-M, IBS-C, IBS-D, and control (HV) subjects.

FIG. 11 illustrate the results of real time quantitative PCR for five targeted genes (SERT, TPH1, MAO-A, TLR4, and TLR7) in samples from IBS-M, IBS-C, IBS-D, and control (HV) subjects.

The figures from US Patent Publication No. 2008/0085524, filed Aug. 14, 2007, are herein incorporated by reference in their entirety for all purposes.

### DETAILED DESCRIPTION OF THE INVENTION

### I. Introduction

Irritable bowel disease (IBS) is a highly prevalent functional gastrointestinal disorder affecting 15-20% population in Western countries, with a higher prevalence in women. IBS is classified into three groups according to predominant bowel symptoms: constipation predominant IBD (IBS-C), diarrhea predominant IBS (IBS-D), and IBS with alternating symptoms of diarrhea and constipation (IBS-A).

Diagnosing a patient as having IBS can be challenging due to the similarity in symptoms between IBS and other diseases or disorders. For example, patients who have inflammatory bowel disease (IBD), but who exhibit mild signs and symptoms such as bloating, diarrhea, constipation, and abdominal pain can be difficult to distinguish from patients with IBS. As a result, the similarity in symptoms between IBS and IBD renders rapid and accurate diagnosis difficult and hampers early and effective treatment of the disease.

IBS is a diagnosis of exclusion in the current clinical practice. Patients are diagnosed by symptom-based Rome criteria, which are recurrent abdominal pain or discomfort at least 3 days per month for the past 3 months, associated with improvement with defecation and onsets associated with a change in frequency or form of stool. The symptoms are often seen in other GI disorders such as functional dyspepsia, fibromyalgia, chronic pelvic pain, and interstitial cystitis. Existence of co-morbidities further complicates the diagnosis.

While the etiology of this disease remains obscure, there are a body of evidence suggesting several pathophysiological pathways are dysregulated including serotonin biosynthesis and metabolism (Gershon M D., J Clin Gastroenterol 39(5 Suppl 3):S184-93 (2005); Coates M D et al., Gastroenterology 126(7):1657-64 (2004); Mawe GM et al., Aliment Pharmacol Ther 23(8):1067-76 (2006)), mast cell infiltration (Róka R et al., Clin Gastroenterol Hepatol 5(5):550-5 (2007); Barbara G et al., Gastroenterology 2007 January; 132(1):26-37; Guilarte M et al., Gut 56(2):203-9 (2007); Barbara G et al., Gastroenterology 126(3):693-702 (2004); O'Sullivan M et al., Neurogastroenterol Motil 12(5):449-57 (2000)), visceral hypersensitivity, stress response and bacteria infection (post infectious-IBS). Given the multiple potential pathophysiologic etiologies of this phenotypic ally heterogeneous disease, it is unlikely that any single diagnostic test or biomarker will reliably identify subjects with IBS. Moreover, the reluctance of clinicians to rely upon symptom-based criteria to diagnose IBS plus the poor diagnostic values of the currently available tests justify the development of a simple but sensitive and specific assay to assist clinicians to make a confident diagnosis of IBS. Towards this goal, Prometheus Laboratories developed the first blood based test for IBS which consists 10 serum biomarkers and an algorithm. The markers are associated with biochemical or physiological pathways that are involved in gut motility, brain-gut axis, neuronal regulation or immune function. The sensitivity, specificity and accuracy of the Prometheus IBS Diagnostic test are 50%, 88% and 70%, respectively.

The present invention provides, among other aspects, a second generation IBS diagnostic test, employing a candidate gene focus pathway driven approach as well as genome wide gene expression profiling. Gene expression profiling in tissue samples taken from patients with IBS has been reported using sigmoid colonic mucosal tissue (Schmulson M W and Chang L., Am J Med 107(5A): 20S-26S (1999)). However, it is unknown whether there exist “surrogate” transcriptional biomarkers in peripheral blood cells of patients with IBS. Although such gene expression biomarkers have been reported in the literature, however, the markers were derived from data mining of a published inflammatory bowel disease study (Tillisch K and Chang L., Curr Gastroenterol Rep 7(4):249-56 (2005)). Here, we have conducted the first microarray study to identify gene expression biomarkers in peripheral blood samples taken from IBS patients and healthy subjects and the results are presented in this publication.

In current clinical practice, diagnosis of IBS is based on symptoms presented by the patients plus the exclusion of other gastrointestinal disorders. This practice leads clinicians to order a wide variety of tests before making a confident diagnosis of IBS. Unfortunately, most of the tests that clinicians routinely order, including complete blood count, chemistry, liver enzymes, thyroid function studies, and stool sampling, have very low diagnostic values in subjects with typical IBS symptoms and no alarm features (weight loss, blood in the stool, unexplained iron deficiency anemia, nocturnal diarrhea, or a family history of IBD, celiac sprue, or colon cancer) (Cash BD et al., American Journal of Gastroenterology 97(11): 2812-2819 (2002)). Patients are diagnosed by the symptom-based Rome criteria, which are recurrent abdominal pain or discomfort at least 3 days per month for the past 3 months, associated with improvement with defecation and onsets associated with a change in frequency or form of stool. The symptoms are often seen in other GI disorders such as functional dyspepsia, fibromyalgia, chronic pelvic pain, and interstitial cystitis. As a result, patients with IBS visit physicians more often, consume more medications, and undergo more diagnostic tests than nonIBS patients (Schmulson M W and Chang L., Am J Med 107 (5A): 20S-26S (1999); Tillisch K and Chang L., Curr Gastroenterol Rep 7(4):249-56 (2005)). Existence of co-morbidities further complicates the diagnosis. IBS symptoms significantly compromise patient's quality of life and increase health care costs (Spiegel BM et al., Arch Intern Med 164(16):1773-80 (2004)).

A gene expression profile study of IBS patient samples has been reported using sigmoid colon mucosa. Although “surrogate” biomarkers in peripheral blood cells in patients with IBS have been reported previously, the markers were selected by data mining from an existing inflammatory bowel disease study. As such, a need exists for the discovery of novel surrogate biomarkers that will better facilitate the diagnosis of IBS.

The present invention, in one aspect, fulfills this need through the discovery of novel gene expression markers useful for the diagnosis and prognosis of IBS. An Affymetrix microarray study using peripheral whole blood samples from 3 IBS-D, 2 IBS-C patients and 3 healthy volunteers. All IBS patients met Rome III criteria and healthy volunteers had no history of IBS or other active co-morbidities. Unsupervised analysis of the microarray data identified a set of 72 genes that distinguished IBS patients and healthy volunteers. The microarray expression profile of selected genes was further verified by real-time quantitative polymerase chain reaction. Validation of the selected genes was conducted in 22 IBS-C, 17 IBS-D, 12 IBD-M, and 21 healthy volunteers. The expression data was analyzed using Multiple Logistic Regression and Random Forest prediction. In this fashion, a subset of novel predictor genes distinguishing IBS patients from healthy subjects with high accuracy was confirmed. Expression of those genes was further compared in whole blood cells and its matching gut biopsy tissues.

The present invention has important implications for IBS diagnosis. For example, in one aspect of the invention, these novel IBS expression markers can be used for diagnosing, providing a prognosis for, and/or subtyping IBS in a subject in need thereof. In another aspect, these markers can complement the existing symptom-based diagnosis of IBS. In yet another aspect, these markers can be used in combination with other serological markers known in the art for the diagnosis and prognosis of IBS.

### II. Definitions

As used herein, the following terms have the meanings ascribed to them unless specified otherwise.

The term “classifying” includes “to associate” or “to categorize” a sample with a disease state. In certain instances, “classifying” is based on statistical evidence, empirical evidence, or both. In certain embodiments, the methods and systems of classifying use a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “Irritable Bowel Syndrome” or “IBS” includes a group of functional bowel disorders characterized by one or more symptoms including, but not limited to, abdominal pain, abdominal discomfort, change in bowel pattern, loose or more frequent bowel movements, diarrhea, and constipation, typically in the absence of any apparent structural abnormality. There are at least three forms of IBS, depending on which symptom predominates: (1) diarrhea-predominant (IBS-D); (2) constipation-predominant (IBS-C); and (3) IBS with alternating stool pattern (IBS-A). IBS can also occur in the form of a mixture of symptoms (IBS-M). There are also various clinical subtypes of IBS, such as post-infectious IBS (IBS-PI).

The term “sample” includes any biological specimen obtained from an individual. Suitable samples for use in the present invention include, without limitation, whole blood, plasma, serum, saliva, urine, stool, sputum, tears, any other bodily fluid, tissue samples (e.g., biopsy), and cellular extracts thereof (e.g., red blood cellular extract). In a preferred embodiment, the sample is a serum sample. The use of samples such as serum, saliva, and urine is well known in the art (see, e.g., Hashida et al., J. Clin. Lab. Anal., 11:267-86 (1997)). One skilled in the art will appreciate that samples such as serum samples can be diluted prior to the analysis of marker levels.

The term “biomarker” or “marker” includes any diagnostic marker such as a biochemical marker, serological marker, genetic marker, or other clinical or echographic characteristic that can be used to classify a sample from an individual as an IBS sample or to rule out one or more diseases or disorders associated with IBS-like symptoms in a sample from an individual. The term “biomarker” or “marker” also encompasses any classification marker such as a biochemical marker, serological marker, genetic marker, or other clinical or echographic characteristic that can be used to classify IBS into one of its various forms or clinical subtypes. Non-limiting examples of diagnostic markers suitable for use in the present invention are described below and include mRNAs and proteins found in Tables 2 and 3 below (e.g., FOXD3, PI4K2A, MAP1LC3A, ACSS2, ASIP, OR2L8, LPAR5, JARID1B, CDKN1C, etc.). Other examples of diagnostic markers include those described in US Patent Publication No. 2008/0085524, filed Aug. 14, 2007, U.S. Provisional Application Ser. No. 61/220,525, filed Jun. 25, 2009, and U.S. Provisional Application Ser. No. 61/256,717, filed Oct. 30, 2009, all of which are herein incorporated by reference in their entirety for all purposes. In some embodiments, diagnostic markers can be used to classify IBS into one of its various forms or clinical subtypes. In other embodiments, classification markers can be used to classify a sample as an IBS sample or to rule out one or more diseases or disorders associated with IBS-like symptoms. One skilled in the art will know of additional diagnostic and classification markers suitable for use in the present invention.

As used herein, the term “profile” includes any set of data that represents the distinctive features or characteristics associated with a disease or disorder such as IBS or IBD. The term encompasses a “diagnostic marker profile” that analyzes one or more diagnostic markers in a sample, a “symptom profile” that identifies one or more IBS-related clinical factors (i.e., symptoms) an individual is experiencing or has experienced, and combinations thereof. For example, a “diagnostic marker profile” can include a set of data that represents the presence or level of one or more diagnostic markers associated with IBS and/or IBD. In one embodiment, a profile includes an “expression profile” or “nucleic acid profile” comprising a set of data corresponding to the level of expression of a marker or set of markers (e.g., RNAs, mRNAs, miRNAs, non-coding RNAs, proteins, and the like) in a sample taken from a subject. A “gene expression profile” includes a set of gene expression data that represents the RNA, mRNA, miRNA, and/or non-coding RNA levels of one or more genes associated with IBS, IBD, or a subtype thereof. Likewise, a “symptom profile” can include a set of data that represents the presence, severity, frequency, and/or duration of one or more symptoms associated with IBS and/or IBD.

The term “individual,” “subject,” or “patient” typically refers to humans, but also to other animals including, e.g., other primates, rodents, canines, felines, equines, ovines, porcines, and the like.

The term “gene” includes segments of DNA that are transcribed into RNA, including mRNA, miRNA, tRNA, rRNA, non-coding RNA, and the like. The term embraces segments of DNA involved in producing a polypeptide chain as well as regions preceding and following the coding region, such as the promoter, 5′-untranslated region (5′UTR), and 3′-untranslated region (3′UTR), as well as intervening sequences (introns) located between individual coding segments (exons).

The term “nucleic acid” or “polynucleotide” includes deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, splice variants, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, and RNA encoded by a gene (e.g., mRNA, miRNA, tRNA, rRNA, etc.).

The term “polymorphism” include the occurrence of two or more genetically determined alternative sequences or alleles in a population. A “polymorphic site” includes the locus at which divergence occurs. A polymorphic locus can be as small as one base pair (single nucleotide polymorphism, or SNP) or can comprise an insertion or deletion of multiple nucleotides. Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allele is arbitrarily designated as the reference allele and other alleles are designated as alternative or “variant alleles.” The allele occurring most frequently in a selected population is sometimes referred to as the “wild-type” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest.

A “single nucleotide polymorphism” or “SNP” occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

The term “genotype” as used herein includes to the genetic composition of an organism, including, for example, whether a diploid organism is heterozygous or homozygous for one or more variant alleles of interest.

As used herein, the term “substantially the same amino acid sequence” includes an amino acid sequence that is similar, but not identical to, the naturally-occurring amino acid sequence. For example, an amino acid sequence that has substantially the same amino acid sequence as a naturally-occurring peptide, polypeptide, or protein can have one or more modifications such as amino acid additions, deletions, or substitutions relative to the amino acid sequence of the naturally-occurring peptide, polypeptide, or protein, provided that the modified sequence retains substantially at least one biological activity of the naturally-occurring peptide, polypeptide, or protein such as immunoreactivity. Comparison for substantial similarity between amino acid sequences is usually performed with sequences between about 6 and 100 residues, preferably between about 10 and 100 residues, and more preferably between about 25 and 35 residues. A particularly useful modification of a peptide, polypeptide, or protein of the present invention, or a fragment thereof, is a modification that confers, for example, increased stability. Incorporation of one or more D-amino acids is a modification useful in increasing stability of a polypeptide or polypeptide fragment. Similarly, deletion or substitution of lysine residues can increase stability by protecting the polypeptide or polypeptide fragment against degradation.

The term “monitoring the progression or regression of IBS” includes the use of the methods, systems, and code of the present invention to determine the disease state (e.g., presence or severity of IBS) of an individual. In certain instances, the results of an algorithm (e.g., a learning statistical classifier system) are compared to those results obtained for the same individual at an earlier time. In some embodiments, the methods, systems, and code of the present invention can be used to predict the progression of IBS, e.g., by determining a likelihood for IBS to progress either rapidly or slowly in an individual based on an analysis of diagnostic markers and/or the identification or IBS-related symptoms. In other embodiments, the methods, systems, and code of the present invention can be used to predict the regression of IBS, e.g., by determining a likelihood for IBS to regress either rapidly or slowly in an individual based on an analysis of diagnostic markers and/or the identification or IBS-related symptoms.

The term “monitoring drug efficacy in an individual receiving a drug useful for treating IBS” includes the use of the methods, systems, and code of the present invention to determine the effectiveness of a therapeutic agent for treating IBS after it has been administered. In certain instances, the results of an algorithm (e.g., a learning statistical classifier system) are compared to those results obtained for the same individual before initiation of use of the therapeutic agent or at an earlier time in therapy. As used herein, a drug useful for treating IBS is any compound or drug used to improve the health of the individual and includes, without limitation, IBS drugs such as serotonergic agents, antidepressants, chloride channel activators, chloride channel blockers, guanylate cyclase agonists, antibiotics, opioids, neurokinin antagonists, antispasmodic or anticholinergic agents, belladonna alkaloids, barbiturates, glucagon-like peptide-1 (GLP-1) analogs, corticotropin releasing factor (CRF) antagonists, probiotics, free bases thereof, pharmaceutically acceptable salts thereof, derivatives thereof, analogs thereof, and combinations thereof.

The term “therapeutically effective amount or dose” includes a dose of a drug that is capable of achieving a therapeutic effect in a subject in need thereof. For example, a therapeutically effective amount of a drug useful for treating IBS can be the amount that is capable of preventing or relieving one or more symptoms associated with IBS. The exact amount can be ascertainable by one skilled in the art using known techniques (see, e.g., Lieberman, Pharmaceutical Dosage Forms, Vols. 1-3 (1992); Lloyd, The Art, Science and Technology of Pharmaceutical Compounding (1999); Pickar, Dosage Calculations (1999); and Remington: The Science and Practice of Pharmacy, 20th Edition, Gennaro, Ed., Lippincott, Williams & Wilkins (2003)).

### III. Description of the Embodiments

The present invention provides methods, systems, and code for accurately classifying whether a sample from an individual is associated with IBS. In some embodiments, the present invention is useful for classifying a sample from an individual as an IBS sample using a statistical algorithm (e.g., a learning statistical classifier system) and/or empirical data (e.g., the presence or level of an IBS marker). The present invention is also useful for ruling out one or more diseases or disorders that present with IBS-like symptoms and ruling in IBS using a combination of statistical algorithms and/or empirical data. Accordingly, the present invention provides an accurate diagnostic prediction of IBS and prognostic information useful for guiding treatment decisions.

A. Diagnosing IBS

In one aspect, the present invention provides a method for diagnosing Irritable Bowel Syndrome (IBS) in a subject in need thereof, the method comprising: (a) isolating and/or amplifying RNA from a biological sample taken from the subject; (b) contacting the isolated and/or amplified RNA with a detection reagent under conditions suitable to transform the detection reagent into a complex comprising the detection reagent and an IBS RNA biomarker; (c) detecting the level of the complex; and (d) determining if the level of the complex more closely resembles a first reference level associated with IBS or a second reference level associated with an absence of IBS, thereby diagnosing IBS in the subject, wherein the biomarker is an RNA from a gene selected from the group consisting of those found in Table 4. In another embodiment, the gene is selected from the group consisting of those found in Table 6. In a more preferred embodiment, the gene is selected from the group consisting of those found in Table 7 such as at least 1, at least 2 or at least 3, 4, 5, 6, 7, 8, 9, 10, 11, 12, 13, 14, 15, 16, 17, 18, 19, 20, or 26.

In some embodiments of the invention, the IBS RNA biomarker is an mRNA or expressed non-coding RNA. In certain embodiments, the method comprises the detection of at least 2 or at least 3, 4, 5, 6, 7, 8, 9, 10, 11, 12, 13, 14, 15, 16, 17, 18, 19, 20, 25, 30, 35, 40, 45, 50, 55, 60, 65, 70, or more of the genes found in Table 4. In another preferred embodiment, the RNA biomarker(s) are found in Table 6 such as at least 1, at least 2 or at least 3, 4, 5, 6, 7, 8, 9, 10, 11, 12, 13, 14, 15, 16, 17, 18, 19, 20, 25, 30, 35, or 40. In a more preferred embodiment, the RNA biomarker(s) are found in Table 7 such as at least 1, at least 2 or at least 3, 4, 5, 6, 7, 8, 9, 10, 11, 12, 13, 14, 15, 16, 17, 18, 19, 20, or 26.

In one embodiment of the invention, the IBS RNA biomarker is a mRNA molecule encoding a protein having an amino acid sequence of any one of SEQ ID NOS:1 to 75 and 154 to 162. In another embodiment, the IBS RNA biomarker is an RNA molecule comprising a nucleic acid sequence of any one of SEQ ID NOS:76 to 153.

In a particular embodiment, the IBS RNA biomarker is an RNA molecule transcribed from a gene selected from the group consisting of CCDC147, VIPR1, LPAR5, CCDC144A, GNG3, ACSS2, ZNF33B, PMS2L2, RUSC1, ARHGE, ASIP, OR2L8, PI4K2A, and FOXD3. In a preferred embodiment, the gene is CCDC147, VIPR1, LPAR5, CCDC144A, or GNG3. In another embodiment, the method for diagnosing or subtyping IBS comprises detecting a panel of at least about 5 biomarkers. In a preferred embodiment, the markers comprise CCDC147, VIPR1, LPAR5, CCDC144A, and GNG3.

In certain embodiments, the detection reagent comprises an oligonucleotide and the step of detecting the level of the complex (e.g., via transformation) comprises oligonucleotide hybridization (e.g., microarray or bead-based hybridization assays, xMAP assay, northern blot, dot blot, RNase protection assay, and the like) and/or nucleic acid amplification (e.g., PCR, qPCR, RT-PCR, qRT-PCR, mass spectrometry, and the like). In yet other embodiments, the detection reagent is an antibody and the method of determining the level of complex (e.g., transformation) in the sample comprises an immunochemical assay (i.e., immunofluorescence assay, ELISA, IFA, and the like).

The sample used for detecting or determining the presence or level of at least one diagnostic marker is typically whole blood, plasma, serum, saliva, urine, stool (i.e., feces), tears, and any other bodily fluid, or a tissue sample (i.e., biopsy) such as a small intestine or colon sample. Preferably, the sample is serum, whole blood, plasma, stool, urine, or a tissue biopsy. In certain instances, the methods of the present invention further comprise obtaining the sample from the individual prior to detecting or determining the presence or level of at least one diagnostic marker in the sample.

In certain embodiments, the methods of the present invention comprise determining an RNA IBS biomarker profile in combination with an additional protein or serological IBS biomarker. In some embodiments, the additional diagnostic marker profile is determined by detecting the presence or level of at least one, two, three, four, five, six, seven, eight, nine, ten, or more additional diagnostic markers selected from those found in Table 2, those found in US Patent Publication No. 2008/0085524, filed Aug. 14, 2007, U.S. Provisional Application Ser. No. 61/220,525, filed Jun. 25, 2009, and U.S. Provisional Application Ser. No. 61/256,717, filed Oct. 30, 2009.

In some embodiments, a panel for measuring one or more of the diagnostic markers described above may be constructed and used for classifying the sample as an IBS sample, an IBS-subtype sample, or a non-IBS sample. One skilled in the art will appreciate that the presence or level of a plurality of diagnostic markers can be determined simultaneously or sequentially, using, for example, an aliquot or dilution of the individual's sample. In certain instances, the level of a particular diagnostic marker in the individual's sample is considered to be elevated when it is at least about 25%, 50%, 75%, 100%, 125%, 150%, 175%, 200%, 250%, 300%, 350%, 400%, 450%, 500%, 600%, 700%, 800%, 900%, or 1000% greater than the level of the same marker in a comparative sample (e.g., a normal, GI control, IBS, IBD, and/or Celiac disease sample) or population of samples (e.g., greater than a median level of the same marker in a comparative population of normal, GI control, IBS, IBD, and/or Celiac disease samples). In certain other instances, the level of a particular diagnostic marker in the individual's sample is considered to be lowered when it is at least about 5%, 10%, 15%, 20%, 25%, 30%, 35%, 40%, 45%, 50%, 55%, 60%, 65%, 70%, 75%, 80%, 85%, 90%, or 95% less than the level of the same marker in a comparative sample (e.g., a normal, GI control, IBS, IBD, and/or Celiac disease sample) or population of samples (e.g., less than a median level of the same marker in a comparative population of normal, GI control, IBS, IBD, and/or Celiac disease samples). In yet other embodiments, an IBS marker is considered to be differentially expressed when the magnitude its log2 fold change (i.e., positive or negative value) is at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 or greater, with respect to the same marker in a comparative population of normal, GI control, IBS, IBD, and/or Celiac disease samples. In a preferred embodiment, the magnitude of a differentially expressed IBS biomarker is at least about 1.0, more preferably at least about 1.5, and most preferably at least about 2.5.

In some embodiments, the method of ruling in IBS, diagnosing IBS, or classifying IBS comprises determining a diagnostic marker profile optionally in combination with a symptom profile, wherein the symptom profile is determined by identifying the presence or severity of at least one symptom in the individual; and classifying the sample as an IBS sample or non-IBS sample using an algorithm based upon the diagnostic marker profile and the symptom profile. One skilled in the art will appreciate that the diagnostic marker profile and the symptom profile can be determined simultaneously or sequentially in any order.

In some embodiments, classifying a sample as an IBS sample or non-IBS sample is based upon the diagnostic marker profile, alone or in combination with a symptom profile, in conjunction with a statistical algorithm. In certain instances, the statistical algorithm is a learning statistical classifier system. The learning statistical classifier system can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. Preferably, the learning statistical classifier system is a tree-based statistical algorithm (e.g., RF, C&RT, etc.) and/or a NN (e.g., artificial NN, etc.).

In certain instances, the statistical algorithm is a single learning statistical classifier system. Preferably, the single learning statistical classifier system comprises a tree-based statistical algorithm such as a RF or C&RT. As a non-limiting example, a single learning statistical classifier system can be used to classify the sample as an IBS sample or non-IBS sample based upon a prediction or probability value and the presence or level of at least one diagnostic marker (i.e., diagnostic marker profile), alone or in combination with the presence or severity of at least one symptom (i.e., symptom profile). The use of a single learning statistical classifier system typically classifies the sample as an IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In certain other instances, the statistical algorithm is a combination of at least two learning statistical classifier systems. Preferably, the combination of learning statistical classifier systems comprises a RF and a NN, e.g., used in tandem or parallel. As a non-limiting example, a RF can first be used to generate a prediction or probability value based upon the diagnostic marker profile, alone or in combination with a symptom profile, and a NN can then be used to classify the sample as an IBS sample or non-IBS sample based upon the prediction or probability value and the same or different diagnostic marker profile or combination of profiles. Advantageously, the hybrid RF/NN learning statistical classifier system of the present invention classifies the sample as an IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some instances, the data obtained from using the learning statistical classifier system or systems can be processed using a processing algorithm. Such a processing algorithm can be selected, for example, from the group consisting of a multilayer perceptron, backpropagation network, and Levenberg-Marquardt algorithm. In other instances, a combination of such processing algorithms can be used, such as in a parallel or serial fashion.

In certain embodiments, the methods of the present invention further comprise classifying the non-IBS sample as a normal, inflammatory bowel disease (IBD), or non-IBD sample. Classification of the non-IBS sample can be performed, for example, using at least one of the diagnostic markers described above.

In certain other embodiments, the methods of the present invention further comprise sending the IBS classification results to a clinician, e.g., a gastroenterologist or a general practitioner. In another embodiment, the methods of the present invention provide a diagnosis in the form of a probability that the individual has IBS. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having IBS. In yet another embodiment, the methods of the present invention further provide a prognosis of IBS in the individual. For example, the prognosis can be surgery, development of a category or clinic al subtype of IBS, development of one or more symptoms, or recovery from the disease.

In some embodiments, the diagnosis of an individual as having IBS is followed by administering to the individual a therapeutically effective amount of a drug useful for treating one or more symptoms associated with IBS. Suitable IBS drugs include, but are not limited to, serotonergic agents, antidepressants, chloride channel activators, chloride channel blockers, guanylate cyclase agonists, antibiotics, opioid agonists, neurokinin antagonists, antispasmodic or anticholinergic agents, belladonna alkaloids, barbiturates, GLP-1 analogs, CRF antagonists, probiotics, free bases thereof, pharmaceutically acceptable salts thereof, derivatives thereof, analogs thereof, and combinations thereof. Other IBS drugs include bulking agents, dopamine antagonists, carminatives, tranquilizers, dextofisopam, phenytoin, timolol, and diltiazem. Additionally, amino acids like glutamine and glutamic acid which regulate intestinal permeability by affecting neuronal or glial cell signaling can be administered to treat patients with IBS.

In other embodiments, the methods of the present invention further comprise classifying the IBS sample as an IBS-constipation (IBS-C), IBS-diarrhea (IBS-D), IBS-mixed (IBS-M), IBS-alternating (IBS-A), or post-infectious IBS (IBS-PI) sample. In certain instances, the classification of the IBS sample into a category, form, or clinical subtype of IBS is based upon the presence or level of at least one, two, three, four, five, six, seven, eight, nine, ten, or more classification markers. Non-limiting examples of classification markers are described below. Preferably, at least one form of IBS is distinguished from at least one other form of IBS based upon the presence or level of leptin. In certain instances, the methods of the present invention can be used to differentiate an IBS-C sample from an IBS-A and/or IBS-D sample in an individual previously identified as having IBS. In certain other instances, the methods of the present invention can be used to classify a sample from an individual not previously diagnosed with IBS as an IBS-A sample, IBS-C sample, IBS-D sample, or non-IBS sample.

In certain embodiments, the methods further comprise sending the results from the classification to a clinician. In certain other embodiments, the methods further provide a diagnosis in the form of a probability that the individual has IBS-A, IBS-C, IBS-D, IBS-M, or IBS-PI. The methods of the present invention can further comprise administering to the individual a therapeutically effective amount of a drug useful for treating IBS-A, IBS-C, IBS-D, IBS-M, or IBS-PI. Suitable drugs include, but are not limited to, tegaserod (Zelnorm), alosetron (Lotronex®), lubiprostone (Amitiza), rifamixin (Xifaxan), MD-1100, probiotics, and a combination thereof. In instances where the sample is classified as an IBS-A or IBS-C sample and/or the individual is diagnosed with IBS-A or IBS-C, a therapeutically effective dose of tegaserod or other 5-HT₄ agonist (e.g., mosapride, renzapride, AG1-001, etc.) can be administered to the individual. In some instances, when the sample is classified as IBS-C and/or the individual is diagnosed with IBS-C, a therapeutically effective amount of lubiprostone or other chloride channel activator, rifamixin or other antibiotic capable of controlling intestinal bacterial overgrowth, MD-1100 or other guanylate cyclase agonist, asimadoline or other opioid agonist, or talnetant or other neurokinin antagonist can be administered to the individual. In other instances, when the sample is classified as IBS-D and/or the individual is diagnosed with IBS-D, a therapeutically effective amount of alosetron or other 5-HT₃ antagonist (e.g., ramosetron, DDP-225, etc.), crofelemer or other chloride channel blocker, talnetant or other neurokinin antagonist (e.g., saredutant, etc.), or an antidepressant such as a tricyclic antidepressant can be administered to the individual.

In additional embodiments, the methods of the present invention further comprise ruling out intestinal inflammation. Non-limiting examples of intestinal inflammation include acute inflammation, diverticulitis, ileal pouch-anal anastomosis, microscopic colitis, infectious diarrhea, and combinations thereof. In some instances, the intestinal inflammation is ruled out based upon the presence or level of C-reactive protein (CRP), lactoferrin, calprotectin, or combinations thereof.

B. Monitoring IBS

In another aspect, the present invention provides a method for monitoring the progression or regression of Irritable Bowel Syndrome (IBS) in a subject, said method comprising: (a) determining a first biomarker profile from a first biological sample taken from the subject at a first point in time; (b) determining a second biomarker profile from a second biological sample taken from the subject at a second point in time; and (c) comparing said first and said second biomarker profiles to (i) determine which biomarker profile most resembles or least resembles a first reference profile associated with IBS, (ii) determine which biomarker profile least resembles or most resembles a second reference profile associated with the absence of IBS, or (iii) determining at least two of the foregoing resemblances, wherein said biomarker profiles comprise information about the expression of at least 2 biomarkers found in Table 4, thereby monitoring progression or regression of IBS in said subject. In another embodiment, the biomarker profiles comprise information about the expression of at least 2 biomarkers found in Table 6 such as at least 3, 4, 5, 6, 7, 8, 9, 10, 11, 12, 13, 14, 15, 16, 17, 18, 19, 20, 25, 30, 35, or 40 In a preferred embodiment, the biomarker profiles comprise information about the expression of at least 2 biomarkers found in Table 7 such as at least 3, 4, 5, 6, 7, 8, 9, 10, 11, 12, 13, 14, 15, 16, 17, 18, 19, 20, or 26.

In some embodiments of the invention, the IBS RNA biomarker is an mRNA or expressed non-coding RNA. In certain embodiments, the method comprises the detection of at least 2 or at least 3, 4, 5, 6, 7, 8, 9, 10, 11, 12, 13, 14, 15, 16, 17, 18, 19, 20, 25, 30, 35, 40, 45, 50, 55, 60, 65, 70, or more of the genes found in Table 4. In another preferred embodiment, the RNA biomarker(s) are found in Table 6 such as at least 1, at least 2 or at least 3, 4, 5, 6, 7, 8, 9, 10, 11, 12, 13, 14, 15, 16, 17, 18, 19, 20, 25, 30, 35, or 40. In a more preferred embodiment, the RNA biomarker(s) are found in Table 7 such as at least 1, at least 2 or at least 3, 4, 5, 6, 7, 8, 9, 10, 11, 12, 13, 14, 15, 16, 17, 18, 19, 20, or 26.

In one embodiment of the invention, the IBS RNA biomarker is a mRNA molecule encoding a protein having an amino acid sequence of any one of SEQ ID NOS:1 to 75 and 154 to 162. In another embodiment, the IBS RNA biomarker is an RNA molecule comprising a nucleic acid sequence of any one of SEQ ID NOS:76 to 153.

In a particular embodiment, the IBS RNA biomarker is an RNA molecule transcribed from a gene selected from the group consisting of CCDC147, VIPR1, LPAR5, CCDC144A, GNG3, ACSS2, ZNF33B, PMS2L2, RUSC1, ARHGE, ASIP, OR2L8, PI4K2A, and FOXD3. In a preferred embodiment, the gene is CCDC147, VIPR1, LPAR5, CCDC144A, or GNG3. In another embodiment, the method for monitoring the progression or regression of IBS in a subject comprises detecting a panel of at least about 5 biomarkers. In a preferred embodiment, the markers comprise CCDC147, VIPR1, LPAR5, CCDC144A, and GNG3.

In certain embodiments, the detection reagent comprises an oligonucleotide and the step of detecting the level of the complex (e.g., via transformation) comprises oligonucleotide hybridization (e.g., microarray or bead-based hybridization assays, xMAP assay, northern blot, dot blot, RNase protection assay, and the like) and/or nucleic acid amplification (e.g., PCR, qPCR, RT-PCR, qRT-PCR, mass spectrometry, and the like). In yet other embodiments, the detection reagent is an antibody and the method of determining the level of complex (e.g., via transformation) in the sample comprises an immunochemical assay (i.e., immunofluorescence assay, ELISA, IFA, and the like).

The sample used for detecting or determining the presence or level of at least one diagnostic marker is typically whole blood, plasma, serum, saliva, urine, stool (i.e., feces), tears, and any other bodily fluid, or a tissue sample (i.e., biopsy) such as a small intestine or colon sample. Preferably, the sample is serum, whole blood, plasma, stool, urine, or a tissue biopsy. In certain instances, the methods of the present invention further comprise obtaining the sample from the individual prior to detecting or determining the presence or level of at least one diagnostic marker in the sample.

In certain embodiments, the methods of the present invention comprise determining an RNA IBS biomarker profile in combination with an additional protein or serological IBS biomarker. In some embodiments, the additional diagnostic marker profile is determined by detecting the presence or level of at least one, two, three, four, five, six, seven, eight, nine, ten, or more additional diagnostic markers selected from those found in Table 2, those found in US Patent Publication No. 2008/0085524, filed Aug. 14, 2007, U.S. Provisional Application Ser. No. 61/220,525, filed Jun. 25, 2009, and U.S. Provisional Application Ser. No. 61/256,717, filed Oct. 30, 2009.

In some embodiments, a panel for measuring one or more of the diagnostic markers described above may be constructed and used for monitoring the progression or regression of IBS in a subject. One skilled in the art will appreciate that the presence or level of a plurality of diagnostic markers can be determined simultaneously or sequentially, using, for example, an aliquot or dilution of the individual's sample. In certain instances, the level of a particular diagnostic marker in the individual's sample is considered to be elevated when it is at least about 25%, 50%, 75%, 100%, 125%, 150%, 175%, 200%, 250%, 300%, 350%, 400%, 450%, 500%, 600%, 700%, 800%, 900%, or 1000% greater than the level of the same marker in a comparative sample (e.g., a normal, GI control, IBS, IBD, and/or Celiac disease sample) or population of samples (e.g., greater than a median level of the same marker in a comparative population of normal, GI control, IBS, IBD, and/or Celiac disease samples).

In one aspect, a method for monitoring the progression or regression of Irritable Bowel Syndrome (IBS) in a subject comprises determining the level or profile of one or more biomarkers at a first point in time and a second point in time and comparing said levels or profiles. In one embodiment, wherein an elevated level or expression of a biomarker is associated with IBS, a decrease in the level of a biomarker in a sample taken from a subject at a second time, as compared to the expression of the biomarker in a sample taken from the subject at a first time, is indicative of regression of IBS in the subject. In another embodiment, wherein an elevated level or expression of a biomarker is associated with IBS, an increase in the level of a biomarker in a sample taken from a subject at a second time, as compared to the expression of the biomarker in a sample taken from the subject at a first time, is indicative of progression of IBS in the subject.

In certain other instances, the level of a particular diagnostic marker in the individual's sample is considered to be lowered when it is at least about 5%, 10%, 15%, 20%, 25%, 30%, 35%, 40%, 45%, 50%, 55%, 60%, 65%, 70%, 75%, 80%, 85%, 90%, or 95% less than the level of the same marker in a comparative sample (e.g., a normal, GI control, IBS, IBD, and/or Celiac disease sample) or population of samples (e.g., less than a median level of the same marker in a comparative population of normal, GI control, IBS, IBD, and/or Celiac disease samples).

In another embodiment, wherein a reduced level or expression of a biomarker is associated with IBS, an increase in the level of a biomarker in a sample taken from a subject at a second time, as compared to the expression of the biomarker in a sample taken from the subject at a first time, is indicative of regression of IBS in the subject. In another embodiment, wherein a reduced level or expression of a biomarker is associated with IBS, a decrease in the level of a biomarker in a sample taken from a subject at a second time, as compared to the expression of the biomarker in a sample taken from the subject at a first time, is indicative of progression of IBS in the subject.

In yet other embodiments, an IBS marker is considered to be differentially expressed when the magnitude its log2 fold change (i.e., positive or negative value) is at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 or greater, with respect to the same marker in a comparative population of normal, GI control, IBS, IBD, and/or Celiac disease samples. In a preferred embodiment, the magnitude of a differentially expressed IBS biomarker is at least about 1.0, more preferably at least about 1.5, and most preferably at least about 2.5.

In some embodiments, the method of monitoring the progression or regression of IBS in a subject comprises determining a diagnostic marker profile optionally in combination with a symptom profile, wherein the symptom profile is determined by identifying the presence or severity of at least one symptom in the individual at a first point in time; identifying the presence or severity of at least one symptom in the individual at a second point in time; comparing the presence or severity of the at least one symptom profile at said first point in time and said second point in time; a determining if there has been progression or regression of IBS in the individual using an algorithm based upon the diagnostic marker profile and the symptom profile. One skilled in the art will appreciate that the diagnostic marker profile and the symptom profile can be determined simultaneously or sequentially in any order.

In some embodiments, the method of monitoring the progression or regression of IBS in a subject comprises determining a diagnostic marker profile optionally in combination with a symptom profile, wherein the symptom profile is determined by identifying the presence or severity of at least one symptom in the individual; and classifying the sample as an IBS sample or non-IBS sample using an algorithm based upon the diagnostic marker profile and the symptom profile. One skilled in the art will appreciate that the diagnostic marker profile and the symptom profile can be determined simultaneously or sequentially in any order.

In some embodiments, monitoring the progression or regression of IBS in a subject is based upon the diagnostic marker profile, alone or in combination with a symptom profile, in conjunction with a statistical algorithm. In certain instances, the statistical algorithm is a learning statistical classifier system. The learning statistical classifier system can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. Preferably, the learning statistical classifier system is a tree-based statistical algorithm (e.g., RF, C&RT, etc.) and/or a NN (e.g., artificial NN, etc.).

In certain instances, the statistical algorithm is a single learning statistical classifier system. Preferably, the single learning statistical classifier system comprises a tree-based statistical algorithm such as a RF or C&RT. As a non-limiting example, a single learning statistical classifier system can be used to monitor the progression or regression of IBS in a subject based upon a prediction or probability value and the presence or level of at least one diagnostic marker (i.e., diagnostic marker profile), alone or in combination with the presence or severity of at least one symptom (i.e., symptom profile). The use of a single learning statistical classifier system typically classifies the sample as a progressing or regressing IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In certain other instances, the statistical algorithm is a combination of at least two learning statistical classifier systems. Preferably, the combination of learning statistical classifier systems comprises a RF and a NN, e.g., used in tandem or parallel. As a non-limiting example, a RF can first be used to generate a prediction or probability value based upon the diagnostic marker profile, alone or in combination with a symptom profile, and a NN can then be used to determine if the sample corresponds to a progression or regression of IBS based upon the prediction or probability value and the same or different diagnostic marker profile or combination of profiles. Advantageously, the hybrid RF/NN learning statistical classifier system of the present invention classifies the sample as a progressing or regressing IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some instances, the data obtained from using the learning statistical classifier system or systems can be processed using a processing algorithm. Such a processing algorithm can be selected, for example, from the group consisting of a multilayer perceptron, backpropagation network, and Levenberg-Marquardt algorithm. In other instances, a combination of such processing algorithms can be used, such as in a parallel or serial fashion.

In certain other embodiments, the methods of the present invention further comprise sending the IBS classification results to a clinician, e.g., a gastroenterologist or a general practitioner. In another embodiment, the methods of the present invention provide a diagnosis in the form of a probability that IBS is progressing or regressing in the subject. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having IBS that is progressing or regressing. In yet another embodiment, the methods of the present invention further provide a prognosis of IBS in the individual. For example, the prognosis can be surgery, development of a category or clinic al subtype of IBS, development of one or more symptoms, or recovery from the disease.

In some embodiments, the diagnosis of an individual as having IBS is followed by administering to the individual a therapeutically effective amount of a drug useful for treating one or more symptoms associated with IBS. Suitable IBS drugs include, but are not limited to, serotonergic agents, antidepressants, chloride channel activators, chloride channel blockers, guanylate cyclase agonists, antibiotics, opioid agonists, neurokinin antagonists, antispasmodic or anticholinergic agents, belladonna alkaloids, barbiturates, GLP-1 analogs, CRF antagonists, probiotics, free bases thereof, pharmaceutically acceptable salts thereof, derivatives thereof, analogs thereof, and combinations thereof. Other IBS drugs include bulking agents, dopamine antagonists, carminatives, tranquilizers, dextofisopam, phenytoin, timolol, and diltiazem. Additionally, amino acids like glutamine and glutamic acid which regulate intestinal permeability by affecting neuronal or glial cell signaling can be administered to treat patients with IBS.

The methods of the present invention can further comprise administering to the individual a therapeutically effective amount of a drug useful for treating IBS-A, IBS-C, IBS-D, IBS-M, or IBS-PI. Suitable drugs include, but are not limited to, tegaserod (Zelnorm), alosetron (Lotronex®), lubiprostone (Amitiza), rifamixin (Xifaxan), MD-1100, probiotics, and a combination thereof. In instances where the sample is classified as an IBS-A or IBS-C sample and/or the individual is diagnosed with IBS-A or IBS-C, a therapeutically effective dose of tegaserod or other 5-HT₄ agonist (e.g., mosapride, renzapride, AG1-001, etc.) can be administered to the individual. In some instances, when the sample is classified as IBS-C and/or the individual is diagnosed with IBS-C, a therapeutically effective amount of lubiprostone or other chloride channel activator, rifamixin or other antibiotic capable of controlling intestinal bacterial overgrowth, MD-1100 or other guanylate cyclase agonist, asimadoline or other opioid agonist, or talnetant or other neurokinin antagonist can be administered to the individual. In other instances, when the sample is classified as IBS-D and/or the individual is diagnosed with IBS-D, a therapeutically effective amount of alosetron or other 5-HT₃ antagonist (e.g., ramosetron, DDP-225, etc.), crofelemer or other chloride channel blocker, talnetant or other neurokinin antagonist (e.g., saredutant, etc.), or an antidepressant such as a tricyclic antidepressant can be administered to the individual.

In one embodiment, the method for monitoring the progression or regression of IBS may comprise monitoring a subject who has been administered a therapy for IBS, for example a subject who has been administered a therapy for IBS during the intervening time between the collection of a first biological sample and the collection of a second biological sample. Accordingly, in one embodiment the method for monitoring the progression or regression of IBS is useful for evaluating the clinical efficacy of a therapy for IBS.

In one embodiment, wherein an elevated level or expression of a biomarker is associated with IBS, a decrease in the level of a biomarker in a sample taken from a subject at a time point after the administration of a therapy for IBS, as compared to the expression of the biomarker in a sample taken from the subject at a time point prior to administration of the therapy, is indicative of the efficacy of the therapy. In another embodiment, wherein an elevated level or expression of a biomarker is associated with IBS, an increase in the level of a biomarker in a sample taken from a subject at a time point after the administration of a therapy for IBS, as compared to the expression of the biomarker in a sample taken from the subject at a time point prior to administration of the therapy, is indicative of the lack of efficacy of the therapy.

In another embodiment, wherein a reduced level or expression of a biomarker is associated with IBS, an increase in the level of a biomarker in a sample taken from a subject at a time point after the administration of a therapy for IBS, as compared to the expression of the biomarker in a sample taken from the subject at a time point prior to administration of the therapy, is indicative of the efficacy of the therapy. In yet another embodiment, wherein a reduced level or expression of a biomarker is associated with IBS, a reduction in the level of a biomarker in a sample taken from a subject at a time point after the administration of a therapy for IBS, as compared to the expression of the biomarker in a sample taken from the subject at a time point prior to administration of the therapy, is indicative of the lack of efficacy of the therapy.

After determining the efficacy of an IBS therapy in a subject, the method may further comprise continued administration of the therapy, in the case that the subject is responsive to the therapy, or alternatively may comprise discontinuing, altering, and/or administering alternative therapy to the subject, in the case that the subject is not responsive to the therapy.

C. Assigning Therapy for IBS

In another aspect, the present invention provides a method for assigning therapy for IBS to a subject in need thereof, the method comprising (a) isolating and/or amplifying RNA from a biological sample taken from the subject; (b) contacting the isolated and/or amplified RNA with a detection reagent under conditions suitable to transform the detection reagent into a complex comprising the detection reagent and an IBS RNA biomarker; (c) detecting the level of the complex; (d) determining if the level of the complex more closely resembles a first reference level associated with IBS or a second reference level associated with an absence of IBS; and (e) assigning therapy for IBS if said level more closely resembles said first reference level associated with IBS, wherein the IBS RNA biomarker is selected from the group consisting of those found in Table 4. In a preferred embodiment, the RNA biomarker(s) are found in Table 6 such as at least 1, at least 2 or at least 3, 4, 5, 6, 7, 8, 9, 10, 11, 12, 13, 14, 15, 16, 17, 18, 19, 20, 25, 30, 35, or 40. In a more preferred embodiment, the RNA biomarker(s) are found in Table 7 such as at least 1, at least 2 or at least 3, 4, 5, 6, 7, 8, 9, 10, 11, 12, 13, 14, 15, 16, 17, 18, 19, 20, or 26.

In some embodiments of the invention, the IBS RNA biomarker is an mRNA or expressed non-coding RNA. In certain embodiments, the method comprises the detection of at least 2 or at least 3, 4, 5, 6, 7, 8, 9, 10, 11, 12, 13, 14, 15, 16, 17, 18, 19, 20, 25, 30, 35, 40, 45, 50, 55, 60, 65, 70, or more of the genes found in Table 4. In a preferred embodiment, the method comprises the detection of at least 2 or at least 3, 4, 5, 6, 7, 8, 9, 10, 11, 12, 13, 14, 15, 16, 17, 18, 19, 20, 25, 30, 35, or 40 of the genes found in Table 6. In a preferred embodiment, the RNA biomarker(s) are found in Table 6 such as at least 1, at least 2 or at least 3, 4, 5, 6, 7, 8, 9, 10, 11, 12, 13, 14, 15, 16, 17, 18, 19, 20, 25, 30, 35, or 40. In a more preferred embodiment, the method comprises the detection of at least 2 or at least 3, 4, 5, 6, 7, 8, 9, 10, 11, 12, 13, 14, 15, 16, 17, 18, 19, 20, 25, or all 26 of the genes found in Table 7.

In one embodiment of the invention, the IBS RNA biomarker is a mRNA molecule encoding a protein having an amino acid sequence of any one of SEQ ID NOS:1 to 75 and 154 to 162. In another embodiment, the IBS RNA biomarker is an RNA molecule comprising a nucleic acid sequence of any one of SEQ ID NOS:76 to 153.

In a particular embodiment, the IBS RNA biomarker is an RNA molecule transcribed from a gene selected from the group consisting of CCDC147, VIPR1, LPAR5, CCDC144A, GNG3, ACSS2, ZNF33B, PMS2L2, RUSC1, ARHGE, ASIP, OR2L8, PI4K2A, and FOXD3. In a preferred embodiment, the gene is CCDC147, VIPR1, LPAR5, CCDC144A, or GNG3. In another embodiment, the method for assigning therapy for IBS to a subject in need thereof comprises detecting a panel of at least about 5 biomarkers. In a preferred embodiment, the markers comprise CCDC147, VIPR1, LPAR5, CCDC144A, and GNG3.

In certain embodiments, the detection reagent comprises an oligonucleotide and the step of detecting the level of the complex (e.g., via transformation) comprises oligonucleotide hybridization (e.g., microarray or bead-based hybridization assays, xMAP assay, northern blot, dot blot, RNase protection assay, and the like) and/or nucleic acid amplification (e.g., PCR, qPCR, RT-PCR, qRT-PCR, mass spectrometry, and the like). In yet other embodiments, the detection reagent is an antibody and the method of determining the level of complex (e.g., via transformation) in the sample comprises an immunochemical assay (i.e., immunofluorescence assay, ELISA, IFA, and the like).

The sample used for detecting or determining the presence or level of at least one diagnostic marker is typically whole blood, plasma, serum, saliva, urine, stool (i.e., feces), tears, and any other bodily fluid, or a tissue sample (i.e., biopsy) such as a small intestine or colon sample. Preferably, the sample is serum, whole blood, plasma, stool, urine, or a tissue biopsy. In certain instances, the methods of the present invention further comprise obtaining the sample from the individual prior to detecting or determining the presence or level of at least one diagnostic marker in the sample.

In certain embodiments, the methods of the present invention comprise determining an RNA IBS biomarker profile in combination with an additional protein or serological IBS biomarker. In some embodiments, the additional diagnostic marker profile is determined by detecting the presence or level of at least one, two, three, four, five, six, seven, eight, nine, ten, or more additional diagnostic markers selected from those found in Table 2, those found in US Patent Publication No. 2008/0085524, filed Aug. 14, 2007, U.S. Provisional Application Ser. No. 61/220,525, filed Jun. 25, 2009, and U.S. Provisional Application Ser. No. 61/256,717, filed Oct. 30, 2009.

In some embodiments, a panel for measuring one or more of the diagnostic markers described above may be constructed and used for assigning therapy for IBS to a subject in need thereof. One skilled in the art will appreciate that the presence or level of a plurality of diagnostic markers can be determined simultaneously or sequentially, using, for example, an aliquot or dilution of the individual's sample. In certain instances, the level of a particular diagnostic marker in the individual's sample is considered to be elevated when it is at least about 25%, 50%, 75%, 100%, 125%, 150%, 175%, 200%, 250%, 300%, 350%, 400%, 450%, 500%, 600%, 700%, 800%, 900%, or 1000% greater than the level of the same marker in a comparative sample (e.g., a normal, GI control, IBS, IBD, and/or Celiac disease sample) or population of samples (e.g., greater than a median level of the same marker in a comparative population of normal, GI control, IBS, IBD, and/or Celiac disease samples). In certain other instances, the level of a particular diagnostic marker in the individual's sample is considered to be lowered when it is at least about 5%, 10%, 15%, 20%, 25%, 30%, 35%, 40%, 45%, 50%, 55%, 60%, 65%, 70%, 75%, 80%, 85%, 90%, or 95% less than the level of the same marker in a comparative sample (e.g., a normal, GI control, IBS, IBD, and/or Celiac disease sample) or population of samples (e.g., less than a median level of the same marker in a comparative population of normal, GI control, IBS, IBD, and/or Celiac disease samples). In yet other embodiments, an IBS marker is considered to be differentially expressed when the magnitude its log2 fold change (i.e., positive or negative value) is at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 or greater, with respect to the same marker in a comparative population of normal, GI control, IBS, IBD, and/or Celiac disease samples. In a preferred embodiment, the magnitude of a differentially expressed IBS biomarker is at least about 1.0, more preferably at least about 1.5, and most preferably at least about 2.5.

In some embodiments, the method of assigning therapy for IBS comprises determining a diagnostic marker profile optionally in combination with a symptom profile, wherein the symptom profile is determined by identifying the presence or severity of at least one symptom in the individual; and assigning therapy for IBS using an algorithm based upon the diagnostic marker profile and the symptom profile. One skilled in the art will appreciate that the diagnostic marker profile and the symptom profile can be determined simultaneously or sequentially in any order.

In some embodiments, assigning therapy for IBS is based upon the diagnostic marker profile, alone or in combination with a symptom profile, in conjunction with a statistical algorithm. In certain instances, the statistical algorithm is a learning statistical classifier system. The learning statistical classifier system can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. Preferably, the learning statistical classifier system is a tree-based statistical algorithm (e.g., RF, C&RT, etc.) and/or a NN (e.g., artificial NN, etc.).

In certain instances, the statistical algorithm is a single learning statistical classifier system. Preferably, the single learning statistical classifier system comprises a tree-based statistical algorithm such as a RF or C&RT. As a non-limiting example, a single learning statistical classifier system can be used to assign therapy for IBS based upon a prediction or probability value and the presence or level of at least one diagnostic marker (i.e., diagnostic marker profile), alone or in combination with the presence or severity of at least one symptom (i.e., symptom profile). The use of a single learning statistical classifier system typically classifies the sample as an IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In certain other instances, the statistical algorithm is a combination of at least two learning statistical classifier systems. Preferably, the combination of learning statistical classifier systems comprises a RF and a NN, e.g., used in tandem or parallel. As a non-limiting example, a RF can first be used to generate a prediction or probability value based upon the diagnostic marker profile, alone or in combination with a symptom profile, and a NN can then be used to assigning therapy for IBS based upon the prediction or probability value and the same or different diagnostic marker profile or combination of profiles. Advantageously, the hybrid RF/NN learning statistical classifier system of the present invention classifies the sample as an IBS sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some instances, the data obtained from using the learning statistical classifier system or systems can be processed using a processing algorithm. Such a processing algorithm can be selected, for example, from the group consisting of a multilayer perceptron, backpropagation network, and Levenberg-Marquardt algorithm. In other instances, a combination of such processing algorithms can be used, such as in a parallel or serial fashion.

In certain other embodiments, the methods of the present invention further comprise sending the assignment of a therapy to a clinician, e.g., a gastroenterologist or a general practitioner. In another embodiment, the methods of the present invention provide therapeutic assignments in the form of a probability that the individual will respond to the particular therapy assigned. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of responding to the therapy. In yet another embodiment, the methods of the present invention further provide a prognosis of therapy in the individual. For example, the prognosis can be surgery, development of a category or clinic al subtype of IBS, development of one or more symptoms, regression of IBS, progression of IBS, or recovery from the disease.

In some embodiments, the assignment of a therapy is followed by administering to the individual a therapeutically effective amount of a drug useful for treating one or more symptoms associated with IBS (i.e., administration of the assigned therapy). Suitable IBS drugs include, but are not limited to, serotonergic agents, antidepressants, chloride channel activators, chloride channel blockers, guanylate cyclase agonists, antibiotics, opioid agonists, neurokinin antagonists, antispasmodic or anticholinergic agents, belladonna alkaloids, barbiturates, GLP-1 analogs, CRF antagonists, probiotics, free bases thereof, pharmaceutically acceptable salts thereof, derivatives thereof, analogs thereof, and combinations thereof. Other IBS drugs include bulking agents, dopamine antagonists, carminatives, tranquilizers, dextofisopam, phenytoin, timolol, and diltiazem. Additionally, amino acids like glutamine and glutamic acid which regulate intestinal permeability by affecting neuronal or glial cell signaling can be administered to treat patients with IBS.

In other embodiments, the methods of the present invention further comprise classifying the IBS sample as an IBS-constipation (IBS-C), IBS-diarrhea (IBS-D), IBS-mixed (IBS-M), IBS-alternating (IBS-A), or post-infectious IBS (IBS-PI) sample. In certain instances, the classification of the IBS sample into a category, form, or clinical subtype of IBS is based upon the presence or level of at least one, two, three, four, five, six, seven, eight, nine, ten, or more classification markers. Non-limiting examples of classification markers are described below. Preferably, at least one form of IBS is distinguished from at least one other form of IBS based upon the presence or level of leptin. In certain instances, the methods of the present invention can be used to differentiate an IBS-C sample from an IBS-A and/or IBS-D sample in an individual previously identified as having IBS. In certain other instances, the methods of the present invention can be used to classify a sample from an individual not previously diagnosed with IBS as an IBS-A sample, IBS-C sample, IBS-D sample, or non-IBS sample.

In certain embodiments, the methods further comprise sending the results from the classification to a clinician. In certain other embodiments, the methods further provide a diagnosis in the form of a probability that the individual has IBS-A, IBS-C, IBS-D, IBS-M, or IBS-PI. The methods of the present invention can further comprise administering to the individual a therapeutically effective amount of a drug useful for treating IBS-A, IBS-C, IBS-D, IBS-M, or IBS-PI. Suitable drugs include, but are not limited to, tegaserod (Zelnorm), alosetron (Lotronex®), lubiprostone (Amitiza), rifamixin (Xifaxan), MD-1100, probiotics, and a combination thereof. In instances where the sample is classified as an IBS-A or IBS-C sample and/or the individual is diagnosed with IBS-A or IBS-C, a therapeutically effective dose of tegaserod or other 5-HT₄ agonist (e.g., mosapride, renzapride, AG1-001, etc.) can be administered to the individual. In some instances, when the sample is classified as IBS-C and/or the individual is diagnosed with IBS-C, a therapeutically effective amount of lubiprostone or other chloride channel activator, rifamixin or other antibiotic capable of controlling intestinal bacterial overgrowth, MD-1100 or other guanylate cyclase agonist, asimadoline or other opioid agonist, or talnetant or other neurokinin antagonist can be administered to the individual. In other instances, when the sample is classified as IBS-D and/or the individual is diagnosed with IBS-D, a therapeutically effective amount of alosetron or other 5-HT₃ antagonist (e.g., ramosetron, DDP-225, etc.), crofelemer or other chloride channel blocker, talnetant or other neurokinin antagonist (e.g., saredutant, etc.), or an antidepressant such as a tricyclic antidepressant can be administered to the individual.

D. Determination of a Symptom Profile

The symptom profile is typically determined by identifying the presence or severity of at least one symptom selected from the group consisting of chest pain, chest discomfort, heartburn, uncomfortable fullness after having a regular-sized meal, inability to finish a regular-sized meal, abdominal pain, abdominal discomfort, constipation, diarrhea, bloating, abdominal distension, negative thoughts or feelings associated with having pain or discomfort, and combinations thereof.

In preferred embodiments, the presence or severity of 1, 2, 3, 4, 5, 6, 7, 8, 9, 10, or more of the symptoms described herein is identified to generate a symptom profile that is useful for diagnosing IBS, ruling in IBS, ruling out IBD, predicting IBS, monitoring the progression or regression of IBS, providing a prognosis for IBS, assigning therapy for IBS, and the like. In certain instances, a questionnaire or other form of written, verbal, or telephone survey is used to produce the symptom profile. The questionnaire or survey typically comprises a standardized set of questions and answers for the purpose of gathering information from respondents regarding their current and/or recent IBS-related symptoms. For instance, Example 13 from US Patent Publication No. 2008/0085524 provides exemplary questions that can be included in a questionnaire for identifying the presence or severity of one or more IBS-related symptoms in the individual.

In certain embodiments, the symptom profile is produced by compiling and/or analyzing all or a subset of the answers to the questions set forth in the questionnaire or survey. In certain other embodiments, the symptom profile is produced based upon the individual's response to the following question: “Are you currently experiencing any symptoms?” The symptom profile generated in accordance with either of these embodiments can be used in combination with a diagnostic marker profile in the algorithmic-based methods described herein to improve the accuracy of diagnosing IBS, ruling in IBS, ruling out IBD, predicting IBS, monitoring the progression or regression of IBS, providing a prognosis for IBS, assigning therapy for IBS, and the like.

### IV. Diagnostic Markers

As provided herein, 66 genes were identified with transcripts detected as present in at least 20% of the samples that varied greater than 2-fold between IBS and normal subjects. Among the most significantly elevated transcripts in IBS were pain, inflammation, and gut permeability related genes, including TACR2, SH3GRL3, MICALL1, Rab7L1, VIPR1. TACR2 is a receptor for the tachykinin neuropeptide substance K (neurokinin A). It is associated with G proteins that activate a phosphatidylinositol-calcium second messenger system. Ibodutant is a tachykinin NK2 receptor (TACR2) antagonist currently under phase II clinical trials for IBS. Serotonin triggers contractions in the rabbit ileum by mediate neuronal excitation. Antagonists for neurokinin (NK1 and NK2) receptors partially blocked the serotonin response. VIPR1 is a receptor for VIP. The activity of this receptor is mediated by G proteins which activate adenylyl cyclase. VIP concentration is elevated in serum of IBS patients. Another interesting DEG is MICALL1, which is a cytoskeletal regulator binding to Rab13. MICALL1/Rab13 interaction has been reported to be involved in integrin trafficking to cell surface. Integrin is an important mediator in leukocyte infiltration to the intestine in an inflammatory condition. Increased leukocyte infiltration has been observed in a subset of IBS patients. While those above genes may have biology relevance to IBS, other DEGs with unknown biology functions were identified, for example, CCDC147 is coiled-coil containing protein with undefined biological functions. Since IBS is a complex disease and its etiology remain to be defined, such gene may warrant future studies for IBS research. The unchanged levels of many other genes in the same family suggested that a specific, rather than global activation of those pathways constituted an important part of the disease signature in IBS peripheral blood.

IBS is not associated with any definitive biochemical, structural, or serologic abnormalities that define its presence. The hallmark feature of IBS is abdominal pain or discomfort associated with altered bowel habits, and, often, the abdominal pain prompts patients to seek medical care. Because the symptoms of IBS are common to a number of other GI conditions, IBS was long considered a “diagnosis of exclusion,” leading to excessive testing of patients with characteristic symptoms. Fortunately, advances in research have increased our understanding of IBS pathophysiology, which enabled the development of biologically relevant biomarkers and the development of consensus guidelines advocating a positive diagnosis of IBS based primarily on the pathways involved in the disease and transcript alteration in IBS patients. The identification of gene expression biomarkers with biological relevance such TACR2 and VIPR1 will further enhance our understanding of the pathogenesis of IBS.

The biomarkers provided herein, identified from peripheral blood cells, do not overlap with the published biomarkers identified using gut biopsy tissues. The general limitations of relying on a surrogate end point or a putative surrogate is the possibility of lacking biological relevance of “surrogate markers”. As an example, change in expression of TACR2 in peripheral blood cells may not correlate with change of TACR2 expression in gut tissue, especially in enteric neuron cells, where the pain response is initiated and transmitted. Although gene expression profiling using intestinal tissues is a better measurement for predicting IBS, it is not a feasible test for diagnosis of IBS. Future studies will be performed to compare expression of selected genes using matching peripheral blood and intestinal biopsy tissues.

In the initial gene chip study, only IBS-C and IBS-D patient samples were used (Example 2). The selected genes were validated in IBS-M patient samples using qPCR (Example 5). The relative expression of individual genes vary among the 3 subtypes of IBS, the overall patterns of most DEGs are consistent among the 3 subtypes. Since clinical assignment of IBS subtypes is straightforward base on symptoms of diarrhea, constipation, or mixed types, the focus of this study is to identify genes which are universally regulated in all 3 subtypes.

IBS presents a diagnostic challenge because symptoms overlap with those of other GI disorders such as inflammatory bowel disease, Celiac disease, biliary tract disease, peptic ulcer disease, colorectal carcinoma. Co-morbidities further complicate the diagnosis. Lack of “Gold Standard” has made it very difficult for developing a diagnostic test. In the examples provided herein, the samples were collected by leading GI physicians specialized in IBS diagnosis and treatment. In order to avoid confounding markers which may be associated with co-morbidities, patients who have other GI disorders and psychiatric diseases were excluded. The samples we used for this study were from “homogenous” IBS patient population.

As clinical pharmacogenomic analysis gain acceptance and become more commonplace in clinical trials, it is increasingly evident that microarrays is commonly used as diagnostic devices. One of the importance issues is to establish a rigorous and numerically based method for reporting expression pattern results from a diagnostic assay and how an associated reference range for that pattern is calculated and reported. In one embodiment, the weighted voting method may be used to collapse expression pattern results from many genes into a single numerical confidence score. On important advantage is that it reports a predictive strength score, indicative of the confidence on the prediction for each patient. In the future average confidence scores collected for the accumulating pool of correctly diagnosed patients and correctly nondiagnosed disease-free individuals could be calculated, and a reference range of values for the particular predictive gene set diagnostic in question, could be reported.

As such, in one embodiment, the present invention establishes that there exists disease associated gene signature in peripheral blood of IBS patients. It is possible that because blood circulates throughout the body, their expression profile may serve as a sensitive indicator and physiological monitor of disease and health.

A. RNA Markers

A variety of diagnostic markers are suitable for use in the methods, systems, and code of the present invention for classifying a sample from an individual as an IBS sample or for ruling out one or more diseases or disorders associated with IBS-like symptoms in a sample from an individual. Examples of diagnostic markers include, without limitation, any of the genes, expressed RNAs, or proteins found differentially expressed in IBS or an IBS-subtype, for example those found in Table 1, Table 4, Table 5, Table 6, or Table 7. In a particular embodiment, a diagnostic marker useful in the methods, systems, and code of the present invention is a gene, expressed RNA, or protein found in Table 1. In a preferred embodiment, a diagnostic marker useful in the methods, systems, and code of the present invention is a gene, expressed RNA, or protein found in Table 6. In a more preferred embodiment, a diagnostic marker useful in the methods, systems, and code of the present invention is a gene, expressed RNA, or protein found in Table 7. In one embodiment of the invention, the biomarker is an mRNA molecule encoding a protein having an amino acid sequence of any one of SEQ ID NOS:1 to 75 and 154 to 162. In a related embodiment, the biomarker is an RNA molecule comprising a nucleic acid sequence of any one of SEQ ID NOS:76 to 153.

In a preferred embodiment of the invention, an IBS RNA biomarker comprises an RNA (e.g., mRNA) expressed from a gene selected from CCDC147, VIPR1, LPAR5, CCDC144A, GNG3, ACSS2, ZNF33B, PMS2L2, RUSC1, ARHGE, ASIP, OR2L8, PI4K2A, and FOXD3. In a related embodiment, the biomarker may be a protein or polypeptide encoded by a gene selected from those found in Table 4. In a preferred embodiment, the biomarker may be a protein or polypeptide encoded by a gene selected from those found in Table 6. In a more preferred embodiment, the biomarker may be a protein or polypeptide encoded by a gene selected from those found in Table 7. In a particular embodiment, the protein is encoded by a gene selected from CCDC147, VIPR1, LPAR5, CCDC144A, GNG3, ACSS2, ZNF33B, PMS2L2, RUSC1, ARHGE, ASIP, OR2L8, PI4K2A, and FOXD3.

In a most preferred embodiment, a biomarker of the invention is encoded by a gene selected from CCDC147, VIPR1, LPAR5, CCDC144A, and GNG3. In one embodiment, the methods of the invention comprise the detection of at least two, three, four, or all of CCDC147, VIPR1, LPAR5, CCDC144A, and GNG3. In certain embodiments, the biomarker is an RNA (e.g., mRNA). In other embodiments, the biomarker is a protein or polypeptide encoded by an IBS RNA biomarker.

### 1. CCDC147 Coiled-Coil Domain Containing 147 (CCDC147)

CCDC147 is a 104 kDa protein (NP_{—}001008723 (SEQ ID NO:144)) encoded by the CCDC147 gene (Entrez GeneID: 159686; NM_{—}001008723 (SEQ ID NO:75)). Little is known about the biology of CCDC147. qRT-PCR validation studies of peripheral blood samples from 98 patients with IBS indicate that CCDC147 is highly predictive of IBS, and in particular of the IBS-D subtype (Example 4). In certain embodiments, CCDC147 and/or an mRNA encoding CCDC147 are useful biomarkers for IBS.

In certain instances, the presence or level of CCDC147 or a precursor thereof, is detected at the level of mRNA expression with an assay (e.g., via transformation) such as, e.g., a hybridization assay, an amplification-based assay, e.g. qPCR assay, RT-PCR assay, or a mass spectrometry based assay. In certain other instances, the presence or level of CCDC147 is detected at the level of protein expression (e.g., via transformation) using, e.g., an immunoassay (e.g., ELISA), an immunohistochemical assay, or a mass spectrometry based assay.

### 2. Vasoactive Intestinal Peptide Receptor 1 (VIPR1)

VIPR1 is a 7 transmembrane domain neuropeptide receptor that interacts with the vasoative intestinal peptide (VIP). VIPR1 is found in a number of tissues including brain, peripheral blood leukocytes, and small intestine. Notably, VIP induces smooth muscle relaxation, causes inhibition of gastric acids secretion and absorption from the intestinal lumen, and stimulates the secretion of water into pancreatic juice and bile. VIPR1 is a 48.5 kDa transmembrane protein encoded by the vasoactive intestinal peptide receptor 1 gene (Entrez GeneID: 7433; NM_{—}004624 (SEQ ID NO:58)) and is produced after processing of the vasoactive intestinal peptide receptor 1 precursor polypeptide (NP_{—}004615 (SEQ ID NO:127)). qRT-PCR validation studies of peripheral blood samples from 98 patients with IBS indicate that VIPR1 is highly predictive of IBS, and in particular of the IBS-D subtype (Example 4). In certain embodiments, VIPR1, a VIPR1 precursor protein, and/or an mRNA encoding VIPR1 are useful biomarkers for IBS.

In certain instances, the presence or level of VIPR1 is detected at the level of mRNA expression (e.g., via transformation) with an assay such as, e.g., a hybridization assay, an amplification-based assay, e.g. qPCR assay, RT-PCR assay, or a mass spectrometry based assay. In certain other instances, the presence or level of VIPR1, or a precursor thereof, is detected at the level of protein expression using, e.g., an immunoassay (e.g., ELISA), an immunohistochemical assay, or a mass spectrometry based assay. Suitable ELISA kits for determining the presence or level of VIPR1 in a serum, plasma, saliva, or urine sample are available from, e.g., Sigma-Aldrich (St. Louis, Mo.), US Biological (Swampscott, Mass.), and Novus Biologicals (Littleton, Colo.).

### 3. Lysophosphatidic Acid Receptor 5 (GPR98; LPAR5)

LPAR5 is a 7 transmembrane domain G protein-coupled receptor that transmits extracellular signals from lysophosphatidic acid to cells through heterotrimeric G proteins. LPAR5 interacts with a number of signaling molecules including farnesyl pyrophosphate (FPP), N-arachidonylglycine (NAG), and lysophosphatidic acid. LPAR is a 41.3 kDa transmembrane protein (NP_{—}065133 (SEQ ID NOS:84 and 85)) that is encoded by the lysophosphatidic acid receptor 5 gene (Entrez GeneID: 57121; NM_{—}020400 (SEQ ID NO:9); NM_{—}001142961 (SEQ ID NO:10)). qRT-PCR validation studies of peripheral blood samples from 98 patients with IBS indicate that LPAR5 is highly predictive of IBS, and in particular of the IBS-D subtype (Example 4). In certain embodiments, LPAR5 and/or an mRNA encoding LPAR5 are useful biomarkers for IBS.

In certain instances, the presence or level of LPAR5 or a precursor thereof, is detected at the level of mRNA expression (e.g., via transformation) with an assay such as, e.g., a hybridization assay, an amplification-based assay, e.g. qPCR assay, RT-PCR assay, or a mass spectrometry based assay. In certain other instances, the presence or level of LPAR5 is detected at the level of protein expression using, e.g., an immunoassay (e.g., ELISA), an immunohistochemical assay, or a mass spectrometry based assay. Suitable ELISA kits for determining the presence or level of LPAR5 in a serum, plasma, saliva, or urine sample are available from, e.g., Sigma-Aldrich (St. Louis, Mo.), Abcam (Cambridge, Mass.), and Novus Biologicals (Littleton, Colo.).

### 4. Coiled-Coil Domain Containing 144A (CCDC144A)

CCDC144A is a 165 kDa protein (NP_{—}055510 (SEQ ID NO:103)) encoded by the CCDC147 gene (Entrez GeneID: 9720; NM_{—}014695 (SEQ ID NO:28)). Little is known about the biology of CCDC144A. qRT-PCR validation studies of peripheral blood samples from 98 patients with IBS indicate that CCDC144A is highly predictive of IBS, and in particular of the IBS-D subtype (Example 4). In certain embodiments, CCDC144A and/or an mRNA encoding CCDC144A are useful biomarkers for IBS.

In certain instances, the presence or level of CCDC144A or a precursor thereof, is detected at the level of mRNA expression (e.g., via transformation) with an assay such as, e.g., a hybridization assay, an amplification-based assay, e.g. qPCR assay, RT-PCR assay, or a mass spectrometry based assay. In certain other instances, the presence or level of CCDC144A is detected at the level of protein expression using, e.g., an immunoassay (e.g., ELISA), an immunohistochemical assay, or a mass spectrometry based assay.

### 5. Guanine Nucleotide-Binding Protein G(I)/G(S)/G(O) Subunit Gamma-3 (GNG3)

GNG3 is a gamma subunit for a heterotrimeric G protein. GNG3 provides specificity for the interaction between the heterotrimeric G protein and the G protein receptor (GPR). GNG3 is encoded by the guanine nucleotide binding protein (G protein), gamma 3 gene (Entrez GeneID: 2785; NM_{—}012202 (SEQ ID NO:16)) and is produced after processing of the guanine nucleotide binding protein (G protein), gamma 3 precursor polypeptide (NP_{—}036334 (SEQ ID NO:91)). qRT-PCR validation studies of peripheral blood samples from 98 patients with IBS indicate that GNG3 is highly predictive of IBS, and in particular of the IBS-D subtype (Example 4). In certain embodiments, GNG3, a GNG3 precursor polypeptide, and/or an mRNA encoding GNG3 are useful biomarkers for IBS.

In certain instances, the presence or level of GNG3 is detected at the level of mRNA expression (e.g., via transformation) with an assay such as, e.g., a hybridization assay, an amplification-based assay, e.g. qPCR assay, RT-PCR assay, or a mass spectrometry based assay. In certain other instances, the presence or level of GNG3, or a precursor thereof, is detected at the level of protein expression using, e.g., an immunoassay (e.g., ELISA), an immunohistochemical assay, or a mass spectrometry based assay. Suitable ELISA kits for determining the presence or level of GNG3 in a serum, plasma, saliva, or urine sample are available from, e.g., Sigma-Aldrich (St. Louis, Mo.), Abcam (Cambridge, Mass.), and Novus Biologicals (Littleton, Colo.).

TABLE 1
Differentially expressed genes selected for validation via qRT-PCR.

| | | Gene | |
|---|---|---|---|
| Category/Gene group | Gene Name | Symbol | Assay ID |
| Transcription factor | forkhead box D3 | FOXD3 | Hs00255287_s1 |
| Molecular function unclassified | phosphatidylinositol 4-kinase type 2 | PI4K2A | Hs00218300_m1 |
| | alpha | | |
| Synthase and synthetase | acyl-CoA synthetase short-chain | ACSS2 | Hs00218766_m1 |
| | family member 2 | | |
| Signaling molecule | agouti signaling protein, nonagouti | ASIP | Hs00181770_m1 |
| | homolog (mouse) | | |
| G-protein coupled receptor | olfactory receptor, family 2, | OR2L8 | Hs02338632_g1 |
| | subfamily L, member 8 | | |
| G-protein coupled receptor | lysophosphatidic acid receptor 5 | LPAR5 | Hs01051307_m1 |
| Zinc finger transcription factor | jumonji, AT rich interactive domain | JARID1B | Hs00981910_m1 |
| | 1B | | |
| Kinase modulator | cyclin-dependent kinase inhibitor | CDKN1C | Hs00175938_m1 |
| | 1C (p57, Kip2) | | |
| Molecular function unclassified | coiled-coil domain containing 147 | CCDC147 | Hs01001247_m1 |
| G-protein select regulatory | guanine nucleotide binding protein | GNG3 | Hs00360009_g1 |
| molecule | (G protein), gamma 3 | | |
| Molecular function unclassified | Rho guanine nucleotide exchange | ARHGEF10 | Hs00744267_s1 |
| | factor (GEF) 10 | | |
| Carbohydrate transporter | chromosome 20 open reading frame | C20orf71 | Hs00420455_m1 |
| | 71 | | |
| Carbohydrate transporter | chromosome 20 open reading frame | C20orf114 | Hs01113243_m1 |
| | 114 | | |
| Molecular function unclassified | sushi domain containing 4 | SUSD4 | Hs00215864_m1 |
| Zinc finger transcription factor | zinc finger protein 33B | ZNF33B | Hs00300609_s1 |
| G-protein coupled receptor | olfactory receptor, family 10, | OR10W1 | Hs01398519_s1 |
| | subfamily W, member 1 | | |
| Molecular function unclassified | RCSD domain containing 1 | RCSD1 | Hs00364590_m1 |
| Transcription factor | coiled-coil domain containing 144 | CCDC144A | Hs00417617_m1 |
| | family | | |
| Molecular function unclassified | postmeiotic segregation increased 2- | PMS2L2 | Hs02379621_u1 |
| | like 2 pseudogene | | |
| Signaling molecule | attractin-like 1 | ATRNL1 | Hs00390459_m1 |
| Receptor/G-protein coupled | olfactory receptor, family 51, | OR51E1 | Hs00379183_m1 |
| receptor | subfamily E, member 1 | | |
| Signaling molecule/Peptide | islet amyloid polypeptide | IAPP | Hs00169095_m1 |
| hormone | | | |
| Molecular function unclassified | leucine rich repeat containing 18 | LRRC18 | Hs00736427_m1 |
| Molecular function unclassified | lysophosphatidylglycerol | SNORD77 | Hs00360353_m1 |
| | acyltransferase 1 | | |
| Molecular function unclassified | ring finger protein 26 | RNF26 | Hs00259249_s1 |
| Cell junction protein | gap junction protein, alpha 8, 50 kDa | GJA8 | Hs01102028_m1 |
| Extracellular matrix glycoprotein | glypican 2 | GPC2 | Hs00415099_m1 |
| Select regulatory molecule/ | angiotensinogen (serpin peptidase | AGT | Hs00174854_m1 |
| Protease inhibitor | inhibitor, clade A, member 8) | | |
| Select regulatory molecule/G- | dynamin 3 | DNM3 | Hs00399015_m1 |
| protein | | | |
| Molecular function unclassified | ribosomal RNA processing 7 | RRP7A | Hs00414229_m1 |
| | homolog A (S. cerevisiae) | | |
| Calmodulin related protein | ankyrin repeat domain 5 | ANKRD5 | Hs00223080_m1 |
| Receptor | glycoprotein A33 (transmembrane) | GPA33 | Hs00170690_m1 |
| Molecular function unclassified | RUN and SH3 domain containing 1 | RUSC1 | Hs00204904_m1 |
| Molecular function unclassified | cell division cycle 123 homolog (S. cerevisiae) | CDC123 | Hs00195709_m1 |
| Receptor/G-protein coupled | vasoactive intestinal peptide | VIPR1 | Hs00270351_m1 |
| receptor | receptor 1 | | |
| Transcription factor/Zinc finger | metastasis associated 1 family, | MTA2 | Hs00191018_m1 |
| transcription factor | member 2 | | |
| Molecular function unclassified | ring finger and CCCH-type zinc | RC3H1 | Hs02577215_m1 |
| | finger domains 1 | | |
| Molecular function unclassified | KIAA0090 | KIAA0090 | Hs01076375_m1 |
| Receptor/G-protein coupled | G protein-coupled receptor 87 | GPR87 | Hs00225057_m1 |
| receptor | | | |
| Molecular function unclassified | MAP6 domain containing 1 | MAP6D1 | Hs00227533_m1 |

B. Additional Diagnostic Markers

In certain embodiments, the methods of the present invention comprise determining an RNA IBS biomarker profile in combination with an additional protein or serological IBS biomarker. In some embodiments, the additional diagnostic marker profile is determined by detecting the presence or level of at least one, two, three, four, five, six, seven, eight, nine, ten, or more diagnostic markers selected from the group consisting of a cytokine (e.g., IL-8, IL-β, TWEAK, leptin, OPG, MIP-β, GROα, CXCL4/PF-4, and/or CXCL7/NAP-2), growth factor (e.g., EGF, VEGF, PEDF, BDNF, and/or SDGF), anti-neutrophil antibody (e.g., ANCA, pANCA, cANCA, NSNA, and/or SAPPA), ASCA (e.g., ASCA-IgA, ASCA-IgG, and/or ASCA-IgM), antimicrobial antibody (e.g., anti-OmpC antibody, anti-flagellin antibody, and/or anti-I2 antibody), mast cell marker (e.g., tryptase, histamine, and/or prostaglandin E2 (PGE2)), stress marker (e.g., Urocortin (Ucn), Corticotropin-releasing hormone-binding protein (CRFBP), Cortisol, and/or Adrenocorticotropic hormone (ACTH, corticotrophin)), gastrointestinal hormone (e.g., Calcitonin gene-related peptide (CGRP), Substance P, Nerve Growth Factor (NGF), Neurokinin A, Neurokinin B, Vasoactive Intestinal Peptide (VI P), Glucagon-Like Peptide 2 (GLP-2), Motilin, and/or Pituitary Adenylate Cyclase-Activating Peptide (PACAP)), serotonin metabolite (e.g., tryptophan, 5-HT-o-sulfate, serotonin O-sulfate (5-Hydroxytryptamine O-sulfate; 5-HT-o-sulfate), 5-Hydroxyindoleacetic acid (5-HIAA), 5-HT glucuronide (5-HT-GA), or 5-hydroxytrytophol (5-HTOL)), serotonin pathway marker (e.g., UDP-glucuronosyltransferase 1-6 (UGT1A6), serotonin reuptake transporter (SERT), Tryptophan hydroxylase 1 (TPH1), Monoamine oxidase A (MAO-A), Monoamine oxidase B (MAO-B), or Hydroxytryptamine (serotonin) receptor 3A (5-HT3A; 5-HT3R)), carbohydrate deficient transferrin (CDT), lactoferrin, an anti-tissue transglutaminase (tTG) antibody, lipocalin (e.g., NGAL, NGAL/MMP-9 complex), a matrix metalloproteinase (MMP; e.g., MMP-9), a complex of lipocalin and MMP, a tissue inhibitor of metalloproteinases (TIMPs; e.g., TIMP-1), a globulin (e.g., an alpha-globulin, alpha-2-macroglobulin, haptoglobin, and/or orosomucoid), an actin-severing protein (e.g., gelsolin), an 5100 protein (e.g., calgranulin), a fibrinopeptide (i.e., FIBA), calcitonin gene-related peptide (CGRP), a tachykinin (e.g., Substance P), ghrelin, neurotensin, corticotropin-releasing hormone (CRH), elastase, C-reactive protein (CRP), lactoferrin, an anti-lactoferrin antibody, calprotectin, hemoglobin, NOD2/CARD15, serotonin reuptake transporter (SERT), tryptophan hydroxylase-1,5-hydroxytryptamine (5-HT), lactulose, a serine protease (e.g., tryptase such as (β-tryptase), prostaglandin (e.g., PGE₂), histamine, and a combination thereof. In certain embodiments, the additional biomarker is selected from those found in Table 2. Other non-limiting examples of biomarkers suitable for use in the methods of the present invention include those found in US Patent Publication No. 2008/0085524, filed Aug. 14, 2007, U.S. Provisional Application Ser. No. 61/220,525, filed Jun. 25, 2009, and U.S. Provisional Application Ser. No. 61/256,717, filed Oct. 30, 2009. In one embodiment of the invention, the novel RNA IBS biomarkers of the invention may be combined with a diagnostic marker found in Table 2. One skilled in the art will also know of other diagnostic markers suitable for use in the present invention.

TABLE 2
Exemplary diagnostic markers suitable for use in IBS diagnosis,
prognosis, and classification.

| Family | Biomarker |
|---|---|
| Cytokines | CXCL8/IL-8 |
| | IL-1β |
| | TNF-related weak inducer of apoptosis (TWEAK) |
| | Leptin |
| | Osteoprotegerin (OPG) |
| | CCL19/MIP-3β |
| | CXCL1/GRO1/GROα |
| | CXCL4/PF-4 |
| | CXCL7/NAP-2 |
| | INFβ2/IL-6 |
| | IL-12 |
| | CSIF/IL-10 |
| | Tumor necrosis factor-alpha (TNF-α) |
| Growth Factors | Epidermal growth factor (EGF) |
| | Vascular endothelial growth factor (VEGF) |
| | Pigment epithelium-derived factor (PEDF) |
| | Brain-derived neurotrophic factor (BDNF) |
| | Schwannoma-derived growth factor |
| | (SDGF)/amphiregulin |
| Anti-neutrophil | Anti-neutrophil cytoplasmic antibody (ANCA) |
| antibodies | Perinuclear anti-neutrophil cytoplasmic antibody |
| | (pANCA) |
| | Anti-I2 antibody |
| ASCAs | ASCA-IgA |
| | ASCA-IgG |
| Antimicrobial | Anti-outer membrane protein C (OmpC) antibody |
| antibodies | Anti-Cbir-1 flagellin antibody |
| Lipocalin | Neutrophil gelatinase-associated lipocalin (NGAL) |
| MMP | MMP-9 |
| TIMP | TIMP-1 |
| Alpha-globulins | Alpha-2-macroglobulin (α-MG) |
| | Haptoglobin precursor alpha-2 (Hpα2) |
| | Orosomucoid |
| Actin-severing | Gelsolin |
| protein | |
| S100 protein | Calgranulin A/S100A8/MRP-8 |
| Fibrinopeptide | Fibrinopeptide A (FIBA) |
| Others | Lactoferrin |
| | Anti-tissue transglutaminase (tTG) antibody |
| | Carbohydrate Deficient Transferrin (CDT) |
| Stress Markers | Urocortin (Ucn) |
| | Corticotropin-releasing hormone-binding protein |
| | (CRFBP) |
| | Cortisol |
| | Adrenocorticotropic hormone (ACTH, corticotrophin) |
| Mast Cell | Tryptase |
| Markers | Histamine |
| | Prostaglandin E2 (PGE2) |
| Gastrointestinal | Calcitonin gene-related peptide (CGRP) |
| hormones | Substance P |
| | Nerve Growth Factor (NGF) |
| | Neurokinin A |
| | Neurokinin B |
| | Vasoactive Intestinal Peptide (VIP) |
| | Glucagon-Like Peptide 2 (GLP-2) |
| | Motilin |
| | Pituitary Adenylate Cyclase-Activating Peptide |
| | (PACAP) |
| Serotonin | Serotonin |
| Metabolites | Tryptophan |
| | Serotonin O-sulfate (5-Hydroxytryptamine O-sulfate; |
| | 5-HT-o-sulfate) |
| | 5-Hydroxyindoleacetic acid (5-HIAA) |
| | 5-HT glucuronide (5-HT-GA) |
| Serotonin | UDP-glucuronosyltransferase 1-6 (UGT1A6) |
| Pathway | serotonin reuptake transporter (SERT) |
| Markers | Tryptophan hydroxylase 1 (TPH1) |
| | Monoamine oxidase A (MAO-A) |
| | Monoamine oxidase B (MAO-B) |
| | Hydroxytryptamine (serotonin) receptor 3A (5-HT3A; |
| | 5-HT3R) |

C. Classification Markers

A variety of classification markers are suitable for use in the methods, systems, and code of the present invention for classifying IBS into a category, form, or clinical subtype such as, for example, IBS-constipation (IBS-C), IBS-diarrhea (IBS-D), IBS-mixed (IBS-M), IBS-alternating (IBS-A), or post-infectious IBS (IBS-PI). Examples of classification markers include, without limitation, any of the diagnostic mRNA markers described above, as well as e.g., leptin, serotonin reuptake transporter (SERT), tryptophan hydroxylase-1, 5-hydroxytryptamine (5-HT), tryptase, PGE₂, histamine, mucosal protein 8, keratin-8, claudin-8, zonulin, corticotropin-releasing hormone receptor-1 (CRHR1), corticotropin-releasing hormone receptor-2 (CRHR2), and the like.

For instance, Examples 1 and 2 from U.S. Provisional Application Ser. No. 61/220,525, filed Jun. 25, 2009, which is herein incorporated by reference in its entirety for all purposes, illustrate that measuring α-tryptase levels is particularly useful for distinguishing IBS-C patient samples from IBS-A and IBS-D patient samples. Similarly, Example 1 from US Patent Publication No. 2008/0085524, filed Aug. 14, 2007, which is herein incorporated by reference in its entirety for all purposes, illustrates that measuring leptin levels is particularly useful for distinguishing IBS-C patient samples from IBS-A and IBS-D patient samples. In addition, mucosal SERT and tryptophan hydroxylase-1 expression have been shown to be decreased in IBS-C and IBS-D (see, e.g., Gershon, J. Clin. Gastroenterol., 39 (5 Suppl): 5184-193 (2005)). Furthermore, IBS-C patients show impaired postprandial 5-HT release, whereas IBS-PI patients have higher peak levels of 5-HT (see, e.g., Dunlop, Clin Gastroenterol Hepatol., 3:349-357 (2005)).

### V. Assays

Any of a variety of assays, techniques, and kits known in the art can be used to determine the presence or level of one or more markers in a sample to classify whether the sample is associated with IBS.

The present invention relies, in part, on determining the presence or level of at least one marker in a sample obtained from an individual. As used herein, the term “determining the presence of at least one marker” includes determining the presence of each marker of interest by using any quantitative or qualitative assay known to one of skill in the art. In certain instances, qualitative assays that determine the presence or absence of a particular trait, variable, or biochemical or serological substance (e.g., RNA, mRNA, miRNA, protein, or antibody) are suitable for detecting each marker of interest. In certain other instances, quantitative assays that determine the presence or absence of RNA, protein, antibody, or activity are suitable for detecting each marker of interest. As used herein, the term “determining the level of at least one marker” includes determining the level of each marker of interest by using any direct or indirect quantitative assay known to one of skill in the art. In certain instances, quantitative assays that determine, for example, the relative or absolute amount of RNA, mRNA, miRNA, protein, antibody, or activity are suitable for determining the level of each marker of interest. One skilled in the art will appreciate that any assay useful for determining the level of a marker is also useful for determining the presence or absence of the marker.

Analysis of marker mRNA levels using routine techniques such as Northern analysis, reverse-transcriptase polymerase chain reaction (e.g., qRT-PCR, RT-PCR), microarray analysis, Luminex MultiAnalyte Profiling (xMAP) technology or any other methods based on hybridization to a nucleic acid sequence that is complementary to a portion of the marker coding sequence (e.g., slot blot hybridization) are within the scope of the present invention. Applicable PCR amplification techniques are described in, e.g., Ausubel et al., Current Protocols in Molecular Biology, John Wiley & Sons, Inc. New York (1999), Chapter 7 and Supplement 47; Theophilus et al., “PCR Mutation Detection Protocols,” Humana Press, (2002); and Innis et al., PCR Protocols, San Diego, Academic Press, Inc. (1990). General nucleic acid hybridization methods are described in Anderson, “Nucleic Acid Hybridization,” BIOS Scientific Publishers, 1999. Amplification or hybridization of a plurality of transcribed nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of the genotype of a marker such as a genetic marker can be performed using techniques known in the art including, without limitation, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), and sequencing by hybridization (Chee et al., Science, 274:610-614 (1996); Drmanac et al., Science, 260:1649-1652 (1993); Drmanac et al., Nature Biotech., 16:54-58 (1998)). Non-limiting examples of electrophoretic analysis include slab gel electrophoresis such as agarose or polyacrylamide gel electrophoresis, capillary electrophoresis, and denaturing gradient gel electrophoresis. Other methods for genotyping an individual at a polymorphic site in a marker include, e.g., the INVADER® assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis.

As used herein, the term “antibody” includes a population of immunoglobulin molecules, which can be polyclonal or monoclonal and of any isotype, or an immunologically active fragment of an immunoglobulin molecule. Such an immunologically active fragment contains the heavy and light chain variable regions, which make up the portion of the antibody molecule that specifically binds an antigen. For example, an immunologically active fragment of an immunoglobulin molecule known in the art as Fab, Fab′ or F(ab′)₂ is included within the meaning of the term antibody.

Flow cytometry can be used to determine the presence or level of one or more markers in a sample. Such flow cytometric assays, including bead based immunoassays, can be used to determine, e.g., antibody marker levels in the same manner as described for detecting serum antibodies to Candida albicans and HIV proteins (see, e.g., Bishop and Davis, J. Immunol. Methods, 210:79-87 (1997); McHugh et al., J. Immunol. Methods, 116:213 (1989); Scillian et al., Blood, 73:2041 (1989)).

Phage display technology for expressing a recombinant antigen specific for a marker can also be used to determine the presence or level of one or more markers in a sample. Phage particles expressing an antigen specific for, e.g., an antibody marker can be anchored, if desired, to a multi-well plate using an antibody such as an anti-phage monoclonal antibody (Felici et al., “Phage-Displayed Peptides as Tools for Characterization of Human Sera” in Abelson (Ed.), Methods in Enzymol., 267, San Diego: Academic Press, Inc. (1996)).

A variety of immunoassay techniques, including competitive and non-competitive immunoassays, can be used to determine the presence or level of one or more markers in a sample (see, e.g., Self and Cook, Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing and Nashabeh, Electrophoresis, 18:2184-2193 (1997); Bao, J. Chromatogr. B. Biomed. Sci., 699:463-480 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, Calif.; Kit #449430) and can be performed using a Behring Nephelometer Analyzer (Fink et al., J. Clin. Chem. Clin. Biol. Chem., 27:261-276 (1989)).

Antigen capture ELISA can be useful for determining the presence or level of one or more markers in a sample. For example, in an antigen capture ELISA, an antibody directed to a marker of interest is bound to a solid phase and sample is added such that the marker is bound by the antibody. After unbound proteins are removed by washing, the amount of bound marker can be quantitated using, e.g., a radioimmunoassay (see, e.g., Harlow and Lane, Antibodies: A Laboratory Manual, Cold Spring Harbor Laboratory, New York, 1988)). Sandwich ELISA can also be suitable for use in the present invention. For example, in a two-antibody sandwich assay, a first antibody is bound to a solid support, and the marker of interest is allowed to bind to the first antibody. The amount of the marker is quantitated by measuring the amount of a second antibody that binds the marker. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A radioimmunoassay using, for example, an iodine-125 (¹²⁵ I) labeled secondary antibody (Harlow and Lane, supra) is also suitable for determining the presence or level of one or more markers in a sample. A secondary antibody labeled with a chemiluminescent marker can also be suitable for use in the present invention. A chemiluminescence assay using a chemiluminescent secondary antibody is suitable for sensitive, non-radioactive detection of marker levels. Such secondary antibodies can be obtained commercially from various sources, e.g., Amersham Lifesciences, Inc. (Arlington Heights, Ill.).

The immunoassays described above are particularly useful for determining the presence or level of one or more markers in a sample. As a non-limiting example, an ELISA using an IL-8-binding molecule such as an anti-IL-8 antibody or an extracellular IL-8-binding protein (e.g., IL-8 receptor) is useful for determining whether a sample is positive for IL-8 protein or for determining IL-8 protein levels in a sample. A fixed neutrophil ELISA is useful for determining whether a sample is positive for ANCA or for determining ANCA levels in a sample. Similarly, an ELISA using yeast cell wall phosphopeptidomannan is useful for determining whether a sample is positive for ASCA-IgA and/or ASCA-IgG, or for determining ASCA-IgA and/or ASCA-IgG levels in a sample. An ELISA using OmpC protein or a fragment thereof is useful for determining whether a sample is positive for anti-OmpC antibodies, or for determining anti-OmpC antibody levels in a sample. An ELISA using I2 protein or a fragment thereof is useful for determining whether a sample is positive for anti-I2 antibodies, or for determining anti-I2 antibody levels in a sample. An ELISA using flagellin protein (e.g., Cbir-1 flagellin) or a fragment thereof is useful for determining whether a sample is positive for anti-flagellin antibodies, or for determining anti-flagellin antibody levels in a sample. In addition, the immunoassays described above are particularly useful for determining the presence or level of other diagnostic markers in a sample.

Specific immunological binding of the antibody to the marker of interest can be detected directly or indirectly. Direct labels include fluorescent or luminescent tags, metals, dyes, radionuclides, and the like, attached to the antibody. An antibody labeled with iodine-125 (¹²⁵I) can be used for determining the levels of one or more markers in a sample. A chemiluminescence assay using a chemiluminescent antibody specific for the marker is suitable for sensitive, non-radioactive detection of marker levels. An antibody labeled with fluorochrome is also suitable for determining the levels of one or more markers in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)₂ anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat F(ab′)₂ anti-human IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

Quantitative western blotting can also be used to detect or determine the presence or level of one or more markers in a sample. Western blots can be quantitated by well-known methods such as scanning densitometry or phosphorimaging. As a non-limiting example, protein samples are electrophoresed on 10% SDS-PAGE Laemmli gels. Primary murine monoclonal antibodies are reacted with the blot, and antibody binding can be confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art as described, for example, in Parra et al., J. Vasc. Surg., 28:669-675 (1998).

Alternatively, a variety of immunohistochemical assay techniques can be used to determine the presence or level of one or more markers in a sample. The term immunohistochemical assay encompasses techniques that utilize the visual detection of fluorescent dyes or enzymes coupled (i.e., conjugated) to antibodies that react with the marker of interest using fluorescent microscopy or light microscopy and includes, without limitation, direct fluorescent antibody assay, indirect fluorescent antibody (IFA) assay, anticomplement immunofluorescence, avidin-biotin immunofluorescence, and immunoperoxidase assays. An IFA assay, for example, is useful for determining whether a sample is positive for ANCA, the level of ANCA in a sample, whether a sample is positive for pANCA, the level of pANCA in a sample, and/or an ANCA staining pattern (e.g., cANCA, pANCA, NSNA, and/or SAPPA staining pattern). The concentration of ANCA in a sample can be quantitated, e.g., through endpoint titration or through measuring the visual intensity of fluorescence compared to a known reference standard.

Alternatively, the presence or level of a marker of interest can be determined by detecting or quantifying the amount of the purified marker. Purification of the marker can be achieved, for example, by high pressure liquid chromatography (HPLC), alone or in combination with mass spectrometry (e.g., MALDI/MS, MALDI-TOF/MS, SELDI-TOF/MS, tandem MS, etc.). Qualitative or quantitative detection of a marker of interest can also be determined by well-known methods including, without limitation, Bradford assays, Coomassie blue staining, silver staining, assays for radiolabeled protein, and mass spectrometry.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA®, the CENTAUR® (Bayer), and the NICHOLS ADVANTAGE® (Nichols Institute) immunoassay systems. Preferred apparatuses or protein chips perform simultaneous assays of a plurality of markers on a single surface. Particularly useful physical formats comprise surfaces having a plurality of discrete, addressable locations for the detection of a plurality of different markers. Such formats include protein microarrays, or “protein chips” (see, e.g., Ng et al., J Cell Mol. Med., 6:329-340 (2002)) and certain capillary devices (see, e.g., U.S. Pat. No. 6,019,944). In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection. Yet another suitable format for performing simultaneous assays of a plurality of markers is the Luminex MultiAnalyte Profiling (xMAP) technology, previously known as FlowMetrix and LabMAP (Elshal and McCoy, 2006). This is a multiplex bead-based flow cytometric assay that utilizes polystyrene beads that are internally dyed with different intensities of red and infrared fluorophores. The beads can be bound by various capture reagents such as antibodies, oligonucleotides, and peptides, therefore facilitating the quantification of various RNA, mRNA, miRNA, proteins, ligands, and DNA (Fulton et al, 1997; Kingsmore, 2006; Nolan and Mandy, 2006, Vignali, 2000; Ray et al, 2005).

Several markers of interest may be combined into one test for efficient processing of a multiple of samples. In addition, one skilled in the art would recognize the value of testing multiple samples (e.g., at successive time points, etc.) from the same subject. Such testing of serial samples can allow the identification of changes in marker levels over time. Increases or decreases in marker levels, as well as the absence of change in marker levels, can also provide useful information to classify IBS or to rule out diseases and disorders associated with IBS-like symptoms.

A panel for measuring one or more of the markers described above may be constructed to provide relevant information related to the approach of the present invention for classifying a sample as being associated with IBS. Such a panel may be constructed to determine the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more individual markers. The analysis of a single marker or subsets of markers can also be carried out by one skilled in the art in various clinical settings. These include, but are not limited to, ambulatory, urgent care, critical care, intensive care, monitoring unit, inpatient, outpatient, physician office, medical clinic, and health screening settings.

The analysis of markers could be carried out in a variety of physical formats as well. For example, the use of microtiter plates or automation could be used to facilitate the processing of large numbers of test samples. Alternatively, single sample formats could be developed to facilitate treatment and diagnosis in a timely fashion.

### VI. Statistical Algorithms

In some aspects, the present invention provides methods, systems, and code for classifying whether a sample is associated with IBS using a statistical algorithm or process to classify the sample as an IBS sample or non-IBS sample. In other aspects, the present invention provides methods, systems, and code for classifying whether a sample is associated with IBS using a first statistical algorithm or process to classify the sample as a non-IBD sample or IBD sample (i.e., IBD rule-out step), followed by a second statistical algorithm or process to classify the non-IBD sample as an IBS sample or non-IBS sample (i.e., IBS rule-in step). Preferably, the statistical algorithms or processes independently comprise one or more learning statistical classifier systems. As described herein, a combination of learning statistical classifier systems advantageously provides improved sensitivity, specificity, negative predictive value, positive predictive value, and/or overall accuracy for classifying whether a sample is associated with IBS.

The term “statistical algorithm” or “statistical process” includes any of a variety of statistical analyses used to determine relationships between variables. In the present invention, the variables are the presence or level of at least one marker of interest and/or the presence or severity of at least one IBS-related symptom. Any number of markers and/or symptoms can be analyzed using a statistical algorithm described herein. For example, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more biomarkers and/or symptoms can be included in a statistical algorithm. In one embodiment, logistic regression is used. In another embodiment, linear regression is used. In certain instances, the statistical algorithms of the present invention can use a quantile measurement of a particular marker within a given population as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., tertiles, quartile, quintiles, etc.), or their cumulative indices (e.g., quartile sums of marker levels, etc.) as variables in the algorithms (just as with continuous variables).

Preferably, the statistical algorithms of the present invention comprise one or more learning statistical classifier systems. As used herein, the term “learning statistical classifier system” includes a machine learning algorithmic technique capable of adapting to complex data sets (e.g., panel of markers of interest and/or list of IBS-related symptoms) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and http://stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the CART software available from Salford Systems or the Statistical data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistical data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{light} software developed by Thorsten Joachims (Cornell University) or using the LIBSVM software developed by Chih-Chung Chang and Chih-Jen Lin (National Taiwan University).

The learning statistical classifier systems described herein can be trained and tested using a cohort of samples (e.g., serological samples) from healthy individuals, IBS patients, IBD patients, and/or Celiac disease patients. For example, samples from patients diagnosed by a physician, and preferably by a gastroenterologist as having IBD using a biopsy, colonoscopy, or an immunoassay as described in, e.g., U.S. Pat. No. 6,218,129, are suitable for use in training and testing the learning statistical classifier systems of the present invention. Samples from patients diagnosed with IBD can also be stratified into Crohn's disease or ulcerative colitis using an immunoassay as described in, e.g., U.S. Pat. Nos. 5,750,355 and 5,830,675. Samples from patients diagnosed with IBS can be stratified into IBS-constipation (IBS-C), IBS-diarrhea (IBS-D), IBS-mixed (IBS-M), IBS-alternating (IBS-A), or post-infectious IBS (IBS-PI). Samples from patients diagnosed with IBS using a published criteria such as the Manning, Rome I, Rome II, or Rome III diagnostic criteria are suitable for use in training and testing the learning statistical classifier systems of the present invention. Samples from healthy individuals can include those that were not identified as IBD and/or IBS samples. One skilled in the art will know of additional techniques and diagnostic criteria for obtaining a cohort of patient samples that can be used in training and testing the learning statistical classifier systems of the present invention.

As used herein, the term “sensitivity” refers to the probability that a diagnostic method, system, or code of the present invention gives a positive result when the sample is positive, e.g., having IBS. Sensitivity is calculated as the number of true positive results divided by the sum of the true positives and false negatives. Sensitivity essentially is a measure of how well a method, system, or code of the present invention correctly identifies those with IBS from those without the disease. The statistical algorithms can be selected such that the sensitivity of classifying IBS is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In preferred embodiments, the sensitivity of classifying IBS is at least about 90% when a combination of learning statistical classifier systems is used (see, Example 10 from US Patent Publication No. 2008/0085524, which is incorporated herein by reference in its entirety for all purposes) or at least about 85% when a single learning statistical classifier system is used (see, Example 11 from US Patent Publication No. 2008/0085524, which is incorporated herein by reference in its entirety for all purposes).

The term “specificity” refers to the probability that a diagnostic method, system, or code of the present invention gives a negative result when the sample is not positive, e.g., not having IBS. Specificity is calculated as the number of true negative results divided by the sum of the true negatives and false positives. Specificity essentially is a measure of how well a method, system, or code of the present invention excludes those who do not have IBS from those who have the disease. The statistical algorithms can be selected such that the specificity of classifying IBS is at least about 70%, for example, at least about 75%, 80%, 85%, 86%, 87%, 88%, 89%, 90%, 91%, 92%, 93%, 94%, 95%, 96%, 97%, 98%, or 99%. In preferred embodiments, the specificity of classifying IBS is at least about 86% when a combination of learning statistical classifier systems is used (see, Example 10 from US Patent Publication No. 2008/0085524, which is incorporated herein by reference in its entirety for all purposes) or at least about 84% when a single learning statistical classifier system is used (see, Example 11 from US Patent Publication No. 2008/0085524, which is incorporated herein by reference in its entirety for all purposes).

As used herein, the term “negative predictive value” or “NPV” refers to the probability that an individual identified as not having IBS actually does not have the disease. Negative predictive value can be calculated as the number of true negatives divided by the sum of the true negatives and false negatives. Negative predictive value is determined by the characteristics of the diagnostic method, system, or code as well as the prevalence of the disease in the population analyzed. The statistical algorithms can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In preferred embodiments, the negative predictive value of classifying IBS is at least about 87% when a combination of learning statistical classifier systems is used (see, Example 10 from US Patent Publication No. 2008/0085524, which is incorporated herein by reference in its entirety for all purposes).

The term “positive predictive value” or “PPV” refers to the probability that an individual identified as having IBS actually has the disease. Positive predictive value can be calculated as the number of true positives divided by the sum of the true positives and false positives. Positive predictive value is determined by the characteristics of the diagnostic method, system, or code as well as the prevalence of the disease in the population analyzed. The statistical algorithms can be selected such that the positive predictive value in a population having a disease prevalence is in the range of about 80% to about 99% and can be, for example, at least about 80%, 85%, 86%, 87%, 88%, 89%, 90%, 91%, 92%, 93%, 94%, 95%, 96%, 97%, 98%, or 99%. In preferred embodiments, the positive predictive value of classifying IBS is at least about 90% when a combination of learning statistical classifier systems is used (see, Example 10 from US Patent Publication No. 2008/0085524, which is incorporated herein by reference in its entirety for all purposes).

Predictive values, including negative and positive predictive values, are influenced by the prevalence of the disease in the population analyzed. In the methods, systems, and code of the present invention, the statistical algorithms can be selected to produce a desired clinical parameter for a clinical population with a particular IBS prevalence. For example, learning statistical classifier systems can be selected for an IBS prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as a gastroenterologist's office or a general practitioner's office.

As used herein, the term “overall agreement” or “overall accuracy” refers to the accuracy with which a method, system, or code of the present invention classifies a disease state. Overall accuracy is calculated as the sum of the true positives and true negatives divided by the total number of sample results and is affected by the prevalence of the disease in the population analyzed. For example, the statistical algorithms can be selected such that the overall accuracy in a patient population having a disease prevalence is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In preferred embodiments, the overall accuracy of classifying IBS is at least about 80% when a combination of learning statistical classifier systems is used (see, Example 10 from US Patent Publication No. 2008/0085524, which is incorporated herein by reference in its entirety for all purposes).

### VII. Disease Classification System

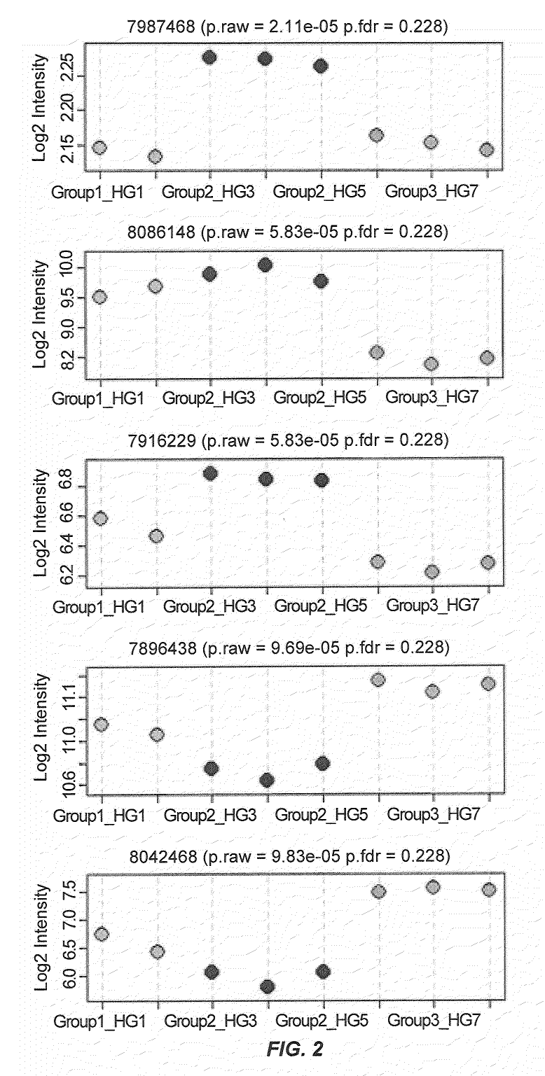

FIG. 2 from US Patent Publication No. 2008/0085524, which is incorporated herein by reference in its entirety for all purposes, illustrates a disease classification system (DCS) (200) according to one embodiment of the present invention. As shown therein, a DCS includes a DCS intelligence module (205), such as a computer, having a processor (215) and memory module (210). The intelligence module also includes communication modules (not shown) for transmitting and receiving information over one or more direct connections (e.g., USB, Firewire, or other interface) and one or more network connections (e.g., including a modem or other network interface device). The memory module may include internal memory devices and one or more external memory devices. The intelligence module also includes a display module (225), such as a monitor or printer. In one aspect, the intelligence module receives data such as patient test results from a data acquisition module such as a test system (250), either through a direct connection or over a network (240). For example, the test system may be configured to run multianalyte tests on one or more patient samples (255) and automatically provide the test results to the intelligence module. The data may also be provided to the intelligence module via direct input by a user or it may be downloaded from a portable medium such as a compact disk (CD) or a digital versatile disk (DVD). The test system may be integrated with the intelligence module, directly coupled to the intelligence module, or it may be remotely coupled with the intelligence module over the network. The intelligence module may also communicate data to and from one or more client systems (230) over the network as is well known. For example, a requesting physician or healthcare provider may obtain and view a report from the intelligence module, which may be resident in a laboratory or hospital, using a client system (230).

The network can be a LAN (local area network), WAN (wide area network), wireless network, point-to-point network, star network, token ring network, hub network, or other configuration. As the most common type of network in current use is a TCP/IP (Transfer Control Protocol and Internet Protocol) network such as the global internetwork of networks often referred to as the “Internet” with a capital “I,” that will be used in many of the examples herein, but it should be understood that the networks that the present invention might use are not so limited, although TCP/IP is the currently preferred protocol.

Several elements in the system shown in FIG. 2 from US Patent Publication No. 2008/0085524 may include conventional, well-known elements that need not be explained in detail here. For example, the intelligence module could be implemented as a desktop personal computer, workstation, mainframe, laptop, etc. Each client system could include a desktop personal computer, workstation, laptop, PDA, cell phone, or any WAP-enabled device or any other computing device capable of interfacing directly or indirectly to the Internet or other network connection. A client system typically runs an HTTP client, e.g., a browsing program, such as Microsoft's Internet Explorer browser, Netscape's Navigator browser, Opera's browser, or a WAP-enabled browser in the case of a cell phone, PDA or other wireless device, or the like, allowing a user of the client system to access, process, and view information and pages available to it from the intelligence module over the network. Each client system also typically includes one or more user interface devices, such as a keyboard, a mouse, touch screen, pen or the like, for interacting with a graphical user interface (GUI) provided by the browser on a display (e.g., monitor screen, LCD display, etc.) (235) in conjunction with pages, forms, and other information provided by the intelligence module. As discussed above, the present invention is suitable for use with the Internet, which refers to a specific global internetwork of networks. However, it should be understood that other networks can be used instead of the Internet, such as an intranet, an extranet, a virtual private network (VPN), a non-TCP/IP based network, any LAN or WAN, or the like.

According to one embodiment, each client system and all of its components are operator configurable using applications, such as a browser, including computer code run using a central processing unit such as an Intel® Pentium® processor or the like. Similarly, the intelligence module and all of its components might be operator configurable using application(s) including computer code run using a central processing unit (215) such as an Intel Pentium processor or the like, or multiple processor units. Computer code for operating and configuring the intelligence module to process data and test results as described herein is preferably downloaded and stored on a hard disk, but the entire program code, or portions thereof, may also be stored in any other volatile or non-volatile memory medium or device as is well known, such as a ROM or RAM, or provided on any other computer readable medium (260) capable of storing program code, such as a compact disk (CD) medium, digital versatile disk (DVD) medium, a floppy disk, ROM, RAM, and the like.

The computer code for implementing various aspects and embodiments of the present invention can be implemented in any programming language that can be executed on a computer system such as, for example, in C, C++, C#, HTML, Java, JavaScript, or any other scripting language, such as VBScript. Additionally, the entire program code, or portions thereof, may be embodied as a carrier signal, which may be transmitted and downloaded from a software source (e.g., server) over the Internet, or over any other conventional network connection as is well known (e.g., extranet, VPN, LAN, etc.) using any communication medium and protocols (e.g., TCP/IP, HTTP, HTTPS, Ethernet, etc.) as are well known.

According to one embodiment, the intelligence module implements a disease classification process for analyzing patient test results and/or questionnaire responses to determine whether a patient sample is associated with IBS. The data may be stored in one or more data tables or other logical data structures in memory (210) or in a separate storage or database system coupled with the intelligence module. One or more statistical processes are typically applied to a data set including test data for a particular patient. For example, the test data might include a diagnostic marker profile, which comprises data indicating the presence or level of at least one marker in a sample from the patient. The test data might also include a symptom profile, which comprises data indicating the presence or severity of at least one symptom associated with IBS that the patient is experiencing or has recently experienced. In one aspect, a statistical process produces a statistically derived decision classifying the patient sample as an IBS sample or non-IBS sample based upon the diagnostic marker profile and/or symptom profile. In another aspect, a first statistical process produces a first statistically derived decision classifying the patient sample as an IBD sample or non-IBD sample based upon the diagnostic marker profile and/or symptom profile. If the patient sample is classified as a non-IBD sample, a second statistical process is applied to the same or a different data set to produce a second statistically derived decision classifying the non-IBD sample as an IBS sample or non-IBS sample. The first and/or the second statistically derived decision may be displayed on a display device associated with or coupled to the intelligence module, or the decision(s) may be provided to and displayed at a separate system, e.g., a client system (230). The displayed results allow a physician to make a reasoned diagnosis or prognosis.

### VIII. Diseases and Disorders with IBS-like Symptoms

A variety of structural or metabolic diseases and disorders can cause signs or symptoms that are similar to IBS. As non-limiting examples, patients with diseases and disorders such as inflammatory bowel disease (IBD), Celiac disease (CD), acute inflammation, diverticulitis, ileal pouch-anal anastomosis, microscopic colitis, chronic infectious diarrhea, lactase deficiency, cancer (e.g., colorectal cancer), a mechanical obstruction of the small intestine or colon, an enteric infection, ischemia, maldigestion, malabsorption, endometriosis, and unidentified inflammatory disorders of the intestinal tract can present with abdominal discomfort associated with mild to moderate pain and a change in the consistency and/or frequency of stools that are similar to IBS. Additional IBS-like symptoms can include chronic diarrhea or constipation or an alternating form of each, weight loss, abdominal distention or bloating, and mucus in the stool.

Most IBD patients can be classified into one of two distinct clinical subtypes, Crohn's disease and ulcerative colitis. Crohn's disease is an inflammatory disease affecting the lower part of the ileum and often involving the colon and other regions of the intestinal tract. Ulcerative colitis is characterized by an inflammation localized mostly in the mucosa and submucosa of the large intestine. Patients suffering from these clinical subtypes of IBD typically have IBS-like symptoms such as, for example, abdominal pain, chronic diarrhea, weight loss, and cramping.

The clinical presentation of Celiac disease is also characterized by IBS-like symptoms such as abdominal discomfort associated with chronic diarrhea, weight loss, and abdominal distension. Celiac disease is an immune-mediated disorder of the intestinal mucosa that is typically associated with villous atrophy, crypt hyperplasia, and/or inflammation of the mucosal lining of the small intestine. In addition to the malabsorption of nutrients, individuals with Celiac disease are at risk for mineral deficiency, vitamin deficiency, osteoporosis, autoimmune diseases, and intestinal malignancies (e.g., lymphoma and carcinoma). It is thought that exposure to proteins such as gluten (e.g., glutenin and prolamine proteins which are present in wheat, rye, barley, oats, millet, triticale, spelt, and kamut), in the appropriate genetic and environmental context, is responsible for causing Celiac disease.

Other diseases and disorders characterized by intestinal inflammation that present with IBS-like symptoms include, for example, acute inflammation, diverticulitis, ileal pouch-anal anastomosis, microscopic colitis, and chronic infectious diarrhea, as well as unidentified inflammatory disorders of the intestinal tract. Patients experiencing episodes of acute inflammation typically have elevated C-reactive protein (CRP) levels in addition to IBS-like symptoms. CRP is produced by the liver during the acute phase of the inflammatory process and is usually released about 24 hours post-commencement of the inflammatory process. Patients suffering from diverticulitis, ileal pouch-anal anastomosis, microscopic colitis, and chronic infectious diarrhea typically have elevated fecal lactoferrin and/or calprotectin levels in addition to IBS-like symptoms. Lactoferrin is a glycoprotein secreted by mucosal membranes and is the major protein in the secondary granules of leukocytes. Leukocytes are commonly recruited to inflammatory sites where they are activated, releasing granule content to the surrounding area. This process increases the concentration of lactoferrin in the stool.

Increased lactoferrin levels are observed in patients with ileal pouch-anal anastomosis (i.e., a pouch is created following complete resection of colon in severe cases of Crohn's disease) when compared to other non-inflammatory conditions of the pouch, like irritable pouch syndrome. Elevated levels of lactoferrin are also observed in patients with diverticulitis, a condition in which bulging pouches (i.e., diverticula) in the digestive tract become inflamed and/or infected, causing severe abdominal pain, fever, nausea, and a marked change in bowel habits. Microscopic colitis is a chronic inflammatory disorder that is also associated with increased fecal lactoferrin levels. Microscopic colitis is characterized by persistent watery diarrhea (non-bloody), abdominal pain usually associated with weight loss, a normal mucosa during colonoscopy and radiological examination, and very specific histopathological changes. Microscopic colitis consists of two diseases, collagenous colitis and lymphocytic colitis. Collagenous colitis is of unknown etiology and is found in patients with long-term watery diarrhea and a normal colonoscopy examination. Both collagenous colitis and lymphocytic colitis are characterized by increased lymphocytes in the lining of the colon. Collagenous colitis is further characterized by a thickening of the sub-epithelial collagen layer of the colon. Chronic infectious diarrhea is an illness that is also associated with increased fecal lactoferrin levels. Chronic infectious diarrhea is usually caused by a bacterial, viral, or protozoan infection, with patients presenting with IBS-like symptoms such as diarrhea and abdominal pain. Increased lactoferrin levels are also observed in patients with IBD.

In addition to determining CRP and/or lactoferrin and/or calprotectin levels, diseases and disorders associated with intestinal inflammation can also be ruled out by detecting the presence of blood in the stool, such as fecal hemoglobin. Intestinal bleeding that occurs without the patient's knowledge is called occult or hidden bleeding. The presence of occult bleeding (e.g., fecal hemoglobin) is typically observed in a stool sample from the patient. Other conditions such as ulcers (e.g., gastric, duodenal), cancer (e.g., stomach cancer, colorectal cancer), and hemorrhoids can also present with IBS-like symptoms including abdominal pain and a change in the consistency and/or frequency of stools.

In addition, fecal calprotectin levels can also be assessed. Calprotectin is a calcium binding protein with antimicrobial activity derived predominantly from neutrophils and monocytes. Calprotectin has been found to have clinical relevance in cystic fibrosis, rheumatoid arthritis, IBD, colorectal cancer, HIV, and other inflammatory diseases. Its level has been measured in serum, plasma, oral, cerebrospinal and synovial fluids, urine, and feces. Advantages of fecal calprotectin in GI disorders have been recognized: stable for 3-7 days at room temperature enabling sample shipping through regular mail; correlated to fecal alpha 1-antitrypsin in patients with Crohn's disease; and elevated in a great majority of patients with gastrointestinal carcinomas and IBD. It was found that fecal calprotectin correlates well with endoscopic and histological gradings of disease activity in ulcerative colitis, and with fecal excretion of indium-111-labelled neutrophilic granulocytes, which is a standard of disease activity in IBD.

In view of the foregoing, it is clear that a wide array of diseases and disorders can cause IBS-like symptoms, thereby creating a substantial obstacle for definitively classifying a sample as an IBS sample. However, the present invention overcomes this limitation by classifying a sample from an individual as an IBS sample using, for example, a statistical algorithm, or by excluding (i.e., ruling out) those diseases and disorders that share a similar clinical presentation as IBS and identifying (i.e., ruling in) IBS in a sample using, for example, a combination of statistical algorithms.

### IX. Therapy and Therapeutic Monitoring

Once a sample from an individual has been classified as an IBS sample, the methods, systems, and code of the present invention can further comprise administering to the individual a therapeutically effective amount of a drug useful for treating one or more symptoms associated with IBS (i.e., an IBS drug). For therapeutic applications, the IBS drug can be administered alone or co-administered in combination with one or more additional IBS drugs and/or one or more drugs that reduce the side-effects associated with the IBS drug.

IBS drugs can be administered with a suitable pharmaceutical excipient as necessary and can be carried out via any of the accepted modes of administration. Thus, administration can be, for example, intravenous, topical, subcutaneous, transcutaneous, transdermal, intramuscular, oral, buccal, sublingual, gingival, palatal, intra-joint, parenteral, intra-arteriole, intradermal, intraventricular, intracranial, intraperitoneal, intralesional, intranasal, rectal, vaginal, or by inhalation. By “co-administer” it is meant that an IBS drug is administered at the same time, just prior to, or just after the administration of a second drug (e.g., another IBS drug, a drug useful for reducing the side-effects of the IBS drug, etc.).

A therapeutically effective amount of an IBS drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of an IBS drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the IBS drug.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an IBS drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An IBS drug can also be formulated into a suppository disposed, for example, in a polyethylene glycol (PEG) carrier.

Liquid dosage forms can be prepared by dissolving or dispersing an IBS drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. An IBS drug can also be formulated into a retention enema.

For topical administration, the therapeutically effective dose can be in the form of emulsions, lotions, gels, foams, creams, jellies, solutions, suspensions, ointments, and transdermal patches. For administration by inhalation, an IBS drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In therapeutic use for the treatment of IBS, an IBS drug can be administered at the initial dosage of from about 0.001 mg/kg to about 1000 mg/kg daily. A daily dose range of from about 0.01 mg/kg to about 500 mg/kg, from about 0.1 mg/kg to about 200 mg/kg, from about 1 mg/kg to about 100 mg/kg, or from about 10 mg/kg to about 50 mg/kg, can be used. The dosages, however, may be varied depending upon the requirements of the individual, the severity of IBS symptoms, and the IBS drug being employed. For example, dosages can be empirically determined considering the severity of IBS symptoms in an individual classified as having IBS according to the methods described herein. The dose administered to an individual, in the context of the present invention, should be sufficient to affect a beneficial therapeutic response in the individual over time. The size of the dose can also be determined by the existence, nature, and extent of any adverse side-effects that accompany the administration of a particular IBS drug in an individual. Determination of the proper dosage for a particular situation is within the skill of the practitioner. Generally, treatment is initiated with smaller dosages which are less than the optimum dose of the IBS drug. Thereafter, the dosage is increased by small increments until the optimum effect under circumstances is reached. For convenience, the total daily dosage may be divided and administered in portions during the day, if desired.

As used herein, the term “IBS drug” includes all pharmaceutically acceptable forms of a drug that is useful for treating one or more symptoms associated with IBS. For example, the IBS drug can be in a racemic or isomeric mixture, a solid complex bound to an ion exchange resin, or the like. In addition, the IBS drug can be in a solvated form. The term “IBS drug” is also intended to include all pharmaceutically acceptable salts, derivatives, and analogs of the IBS drug being described, as well as combinations thereof. For example, the pharmaceutically acceptable salts of an IBS drug include, without limitation, the tartrate, succinate, tartarate, bitartarate, dihydrochloride, salicylate, hemisuccinate, citrate, maleate, hydrochloride, carbamate, sulfate, nitrate, and benzoate salt forms thereof, as well as combinations thereof and the like. Any form of an IBS drug is suitable for use in the methods of the present invention, e.g., a pharmaceutically acceptable salt of an IBS drug, a free base of an IBS drug, or a mixture thereof.

Suitable drugs that are useful for treating one or more symptoms associated with IBS include, but are not limited to, serotonergic agents, antidepressants, chloride channel activators, chloride channel blockers, guanylate cyclase agonists, antibiotics, opioids, neurokinin antagonists, antispasmodic or anticholinergic agents, belladonna alkaloids, barbiturates, glucagon-like peptide-1 (GLP-1) analogs, corticotropin releasing factor (CRF) antagonists, probiotics, free bases thereof, pharmaceutically acceptable salts thereof, derivatives thereof, analogs thereof, and combinations thereof. Other IBS drugs include bulking agents, dopamine antagonists, carminatives, tranquilizers, dextofisopam, phenytoin, timolol, and diltiazem.

Serotonergic agents are useful for the treatment of IBS symptoms such as constipation, diarrhea, and/or alternating constipation and diarrhea. Non-limiting examples of serotonergic agents are described in Cash et al., Aliment. Pharmacol. Ther., 22:1047-1060 (2005), and include 5-HT₃ receptor agonists (e.g., MKC-733, etc.), 5-HT₄ receptor agonists (e.g., tegaserod (Zelnorm), prucalopride, AG1-001, etc.), 5-HT₃ receptor antagonists (e.g., alosetron (Lotronex®), cilansetron, ondansetron, granisetron, dolasetron, ramosetron, palonosetron, E-3620, DDP-225, DDP-733, etc.), mixed 5-HT₃ receptor antagonists/5-HT₄ receptor agonists (e.g., cisapride, mosapride, renzapride, etc.), free bases thereof, pharmaceutically acceptable salts thereof, derivatives thereof, analogs thereof, and combinations thereof. Additionally, amino acids like glutamine and glutamic acid which regulate intestinal permeability by affecting neuronal or glial cell signaling can be administered to treat patients with IBS.

Antidepressants such as selective serotonin reuptake inhibitor (SSRI) or tricyclic antidepressants are particularly useful for the treatment of IBS symptoms such as abdominal pain, constipation, and/or diarrhea. Non-limiting examples of SSRI antidepressants include citalopram, fluvoxamine, paroxetine, fluoxetine, sertraline, free bases thereof, pharmaceutically acceptable salts thereof, derivatives thereof, analogs thereof, and combinations thereof. Examples of tricyclic antidepressants include, but are not limited to, desipramine, nortriptyline, protriptyline, amitriptyline, clomipramine, doxepin, imipramine, trimipramine, maprotiline, amoxapine, clomipramine, free bases thereof, pharmaceutically acceptable salts thereof, derivatives thereof, analogs thereof, and combinations thereof.

Chloride channel activators are useful for the treatment of IBS symptoms such as constipation. A non-limiting example of a chloride channel activator is lubiprostone (Amitiza), a free base thereof, a pharmaceutically acceptable salt thereof, a derivative thereof, or an analog thereof. In addition, chloride channel blockers such as crofelemer are useful for the treatment of IBS symptoms such as diarrhea. Guanylate cyclase agonists such as MD-1100 are useful for the treatment of constipation associated with IBS (see, e.g., Bryant et al., Gastroenterol., 128: A-257 (2005)). Antibiotics such as neomycin can also be suitable for use in treating constipation associated with IBS (see, e.g., Park et al., Gastroenterol., 128: A-258 (2005)). Non-absorbable antibiotics like rifaximin (Xifaxan) are suitable to treat small bowel bacterial overgrowth and/or constipation associated with IBS (see, e.g., Sharara et al., Am. J. Gastroenterol., 101:326-333 (2006)).

Opioids such as kappa opiods (e.g., asimadoline) may be useful for treating pain and/or constipation associated with IBS. Neurokinin (NK) antagonists such as talnetant, saredutant, and other NK2 and/or NK3 antagonists may be useful for treating IBS symptoms such as oversensitivity of the muscles in the colon, constipation, and/or diarrhea. Antispasmodic or anticholinergic agents such as dicyclomine may be useful for treating IBS symptoms such as spasms in the muscles of the gut and bladder. Other antispasmodic or anticholinergic agents such as belladonna alkaloids (e.g., atropine, scopolamine, hyoscyamine, etc.) can be used in combination with barbiturates such as phenobarbital to reduce bowel spasms associated with IBS. GLP-1 analogs such as GTP-010 may be useful for treating IBS symptoms such as constipation. CRF antagonists such as astressin and probiotics such as VSL#3® may be useful for treating one or more IBS symptoms. One skilled in the art will know of additional IBS drugs currently in use or in development that are suitable for treating one or more symptoms associated with IBS.

An individual can also be monitored at periodic time intervals to assess the efficacy of a certain therapeutic regimen once a sample from the individual has been classified as an IBS sample. For example, the levels of certain markers change based on the therapeutic effect of a treatment such as a drug. The patient is monitored to assess response and understand the effects of certain drugs or treatments in an individualized approach. Additionally, patients may not respond to a drug, but the markers may change, suggesting that these patients belong to a special population (not responsive) that can be identified by their marker levels. These patients can be discontinued on their current therapy and alternative treatments prescribed.

### X. Examples

### A. Example 1

The present example demonstrates blood sample collection and RNA isolation there from. Briefly, blood samples were collected from three IBS-D patients, two IBS-C patients, and 3 healthy volunteers. All IBS patients met Rome III criteria and healthy volunteers had no history of IBS or other active co-morbidities. In this case, approximately 2.4 ml of whole blood was collected from each subject. The blood sample was divided into two aliquots, and one was processed according to the leukocyte protocol described above, while the other was collected in the PAXgene system (PreAnalytiX; Hombrechtikon, Switzerland) and processed accordingly.

Because the principal difference between the two techniques is the inclusion of RNA from the erythrocyte fraction, it was investigated whether an overabundance of hemoglobin mRNA might explain the differences in expression between whole blood and leukocyte generated samples. Additional RNA was isolated from whole blood from the healthy subjects using the PAXgene blood collection scheme. Degradation of multiple hemoglobin mRNA species in the samples was accomplished using RNase H and specifically designed primers for nine common hemoglobin genes (Feezor R J et al., Physiol Genomics 19:247-254 (2004)) on four donor samples. Briefly, 5 μg of total cellular RNA was incubated in 10 mM Tris·HCl, pH 7.6, 20 mM KCl with 10 μM of oligonucleotide primers at 70° C. for 5 min. The samples were cooled to 4° C. and 2 U of RNase H (New England Biolabs), along with 20 U of SUPERase Inhibitor (Ambion), was added. The buffer conditions were adjusted to 55 mM Tris·HCl, 85 mM KCl, 3 mM MgCl₂, and 10 mM dithiothreitol, and the samples were incubated at 37° C. for 15 min. Immediately following the incubation, the samples were again cooled to 4° C. and 1 μl of 0.5 M EDTA was added to stop the RNase H digestion. The samples were then repurified using the RNeasy Mini Protocol for RNA Cleanup (Qiagen), according to the manufacturer's specifications and including the optional DNase treatment.

### B. Example 2

The present example demonstrates hybridization of the extracted mRNA samples to an oligonucleotide array. For analysis of the IBD and control serum RNA samples, Affymetric human Gene 1.0 ST arrays (Affymatrix, Santa Clara, Calif.) were used. These arrays are an oligonucleotide-probe based gene array chip containing ˜35,000 transcripts, which provides a comprehensive coverage of the whole human genome.

To prepare the RNA samples for hybridization, eight micrograms of total RNA was used to synthesize cDNA. A T7 promoter sequence introduced during the first strand synthesis was then used to direct cRNA synthesis, which was labeled with biotinylated deoxynucleotide triphosphate, following the manufacturer's protocol (Affymatrix, San Diego, Calif.). After fragmentation, the biotinylated cRNA was hybridized to the gene chip array at 45° C. for 16 h. The chip was washed, stained with phycoerytherin-streptavidin, and scanned with the Gene Chip Scanner 3000. After background correction, preliminary data analysis was done in the Microarray Suite 5.0 software (MAS 5.0, Stratagene, La Jolla, Calif.). For primary analysis PLIER was used as recommended in the work flow of software Gene Spring GX10.0 (Agilent Technologies, Santa. Clara).

### Example 3

The present example describes the data analysis of the microarrays performed, as described in the previous examples. The RNA integrity number (RIN) of each sample and performance of each microarray experiment were analyzed for quality control purposes. The results are given in Table 3.

TABLE 3
RNA sample and microarray quality control matrix.

| | | | Array | Hybridization | Overall |
|---|---|---|---|---|---|
| Array # | Disease | RIN | data QC | control | Notes |
| HG1 | IBS-C | 7.1 | Pass | Pass | Pass |
| HG2 | IBS-C | 5.7 | Pass | Pass | Pass |
| HG3 | IBS-D | 7.1 | Pass | Pass | Pass |
| HG4 | IBS-D | 7.1 | Pass | Pass | Pass |
| HG5 | IBS-D | 7.4 | Pass | Pass | Pass |
| HG6 | HV | 7.9 | Pass | Pass | Pass |
| HG7 | HV | 7.1 | Pass | Pass | Pass |
| HG8 | HV | 8.3 | Pass | Pass | Pass |

Fluorescence intensities for each probe set were uploaded to the Array Assist 6.5 and Gene Spring GX10.0 (Agilent Technologies, Santa Clara) software. Data was normalized by quantitative normalization, and then transferred logarithmically for further analysis to determine changes in a particular gene in IBS patients. In order to compare the changes in gene expression, the data was further normalized by using the 50 RFU fluorescence value as threshold, and statistical analysis showing fold changes was determined (p≦0.05). The top 72 markers that were identified using 2 log2 fold change as a cutoff are shown in Table 4.

TABLE 4
The top 72 gene markers identified using 2 log2 fold changes.

| | | | Log2 | | | FDR | | |
|---|---|---|---|---|---|---|---|---|
| Entrez | Mean | Mean | Fold | t- | Raw | adjusted | | Gene |
| GeneID | Group 2 | Group 3 | Change | statistic | p-value | p-value | Gene Symbol | Descriptions |
| 27022 | 6.71 | 3.98 | −2.73 | −8.51 | 0.00105 | 0.205 | FOXD3 | forkhead box D3 |
| 55361 | 6.63 | 4.06 | −2.57 | −6.63 | 0.00268 | 0.246 | PI4K2A | phosphatidylinositol 4-kinase type 2 |
| | | | | | | | | alpha |
| 84557 | 8.92 | 6.44 | −2.48 | −10.8 | 0.000417 | 0.198 | MAP1LC3A | microtubule-associated protein 1 |
| | | | | | | | | light chain 3 alpha |
| 55902 | 8.92 | 6.44 | −2.48 | −10.8 | 0.000417 | 0.198 | ACSS2 | acyl-CoA synthetase short-chain |
| | | | | | | | | family member 2 |
| 434 | 6.65 | 4.22 | −2.43 | −6.66 | 0.00264 | 0.246 | ASIP | agouti signaling protein; nonagouti |
| | | | | | | | | homolog (mouse) |
| 391190 | 6.13 | 8.47 | 2.34 | 6.56 | 0.00279 | 0.248 | OR2L8 | olfactory receptor; family 2; |
| | | | | | | | | subfamily L; member 8 |
| 57121 | 9.07 | 6.74 | −2.33 | −7.9 | 0.00139 | 0.216 | LPAR5 | lysophosphatidic acid receptor 5 |
| 10765 | 5.48 | 3.19 | −2.29 | −9.01 | 0.00084 | 0.203 | JARID1B | jumonji; AT rich interactive domain |
| | | | | | | | | 1B |
| 1028 | 6.34 | 4.07 | −2.27 | −7.21 | 0.00196 | 0.232 | CDKN1C | cyclin-dependent kinase inhibitor |
| | | | | | | | | 1C (p57; Kip2) |
| *8137081 | 7.61 | 5.53 | −2.08 | −6.49 | 0.00291 | 0.248 | NA | NA |
| 159686 | 5.39 | 7.32 | 1.93 | 9.63 | 0.00065 | 0.198 | CCDC147 | coiled-coil domain containing 147 |
| *8121330 | 5.52 | 7.43 | 1.91 | 6.64 | 0.00267 | 0.246 | NA | NA |
| 441871 | 8.06 | 6.22 | −1.84 | −8.15 | 0.00123 | 0.216 | PRAMEF7 | PRAME family member 7 |
| 2785 | 11 | 9.17 | −1.83 | −9.66 | 0.000642 | 0.198 | GNG3 | guanine nucleotide binding protein |
| | | | | | | | | (G protein); gamma 3 |
| 55160 | 9.78 | 8 | −1.78 | −7.16 | 0.00201 | 0.232 | ARHGEF10L | Rho guanine nucleotide exchange |
| | | | | | | | | factor (GEF) 10-like |
| 440738 | 10.4 | 8.71 | −1.69 | −7.98 | 0.00134 | 0.216 | MAP1LC3C | microtubule-associated protein 1 |
| | | | | | | | | light chain 3 gamma |
| 128861 | 6.38 | 4.69 | −1.69 | −6.4 | 0.00306 | 0.248 | C20orf71 | chromosome 20 open reading frame |
| | | | | | | | | 71 |
| 92747 | 6.38 | 4.69 | −1.69 | −6.4 | 0.00306 | 0.248 | C20orf114 | chromosome 20 open reading frame |
| | | | | | | | | 114 |
| 55061 | 5.82 | 7.5 | 1.68 | 6.91 | 0.0023 | 0.235 | SUSD4 | sushi domain containing 4 |
| 7582 | 7.23 | 8.91 | 1.68 | 7.49 | 0.0017 | 0.223 | ZNF33B | zinc finger protein 33B |
| 81341 | 2.35 | 3.96 | 1.61 | 8.77 | 0.000932 | 0.204 | OR10W1 | olfactory receptor; family 10; |
| | | | | | | | | subfamily W; member 1 |
| 92241 | 10.9 | 9.32 | −1.58 | −8.54 | 0.00103 | 0.205 | RCSD1 | RCSD domain containing 1 |
| 9720 | 9.27 | 7.69 | −1.58 | −6.75 | 0.00251 | 0.244 | CCDC144A | coiled-coil domain containing 144A |
| 5380 | 5.96 | 7.52 | 1.56 | 18.1 | 5.48E−05 | 0.152 | PMS2L2 | postmeiotic segregation increased 2- |
| | | | | | | | | like 2 pseudogene |
| 26033 | 10.9 | 9.37 | −1.53 | −17.3 | 6.55E−05 | 0.152 | ATRNL1 | attractin-like 1 |
| 143503 | 7.43 | 8.96 | 1.53 | 8.21 | 0.0012 | 0.216 | OR51E1 | olfactory receptor; family 51; |
| | | | | | | | | subfamily E; member 1 |
| *8161024 | 10.3 | 8.77 | −1.53 | −9.35 | 0.000729 | 0.2 | NA | NA |
| 391112 | 7.18 | 8.7 | 1.52 | 8.65 | 0.000983 | 0.204 | OR6Y1 | olfactory receptor; family 6; |
| | | | | | | | | subfamily Y; member 1 |
| 3375 | 4.98 | 3.47 | −1.51 | −6.86 | 0.00236 | 0.24 | IAPP | islet amyloid polypeptide |
| *8110666 | 8.94 | 7.45 | −1.49 | −6.95 | 0.00225 | 0.235 | NA | NA |
| 474354 | 11.4 | 9.92 | −1.48 | −8.56 | 0.00102 | 0.205 | LRRC18 | leucine rich repeat containing 18 |
| 692197 | 10.8 | 9.33 | −1.47 | −8.86 | 0.000896 | 0.204 | SNORD77 | small nucleolar RNA; C/D box 77 |
| 9926 | 8.37 | 9.83 | 1.46 | 6.51 | 0.00287 | 0.248 | LPGAT1 | lysophosphatidylglycerol |
| | | | | | | | | acyltransferase 1 |
| 79102 | 7.12 | 8.58 | 1.46 | 10.2 | 0.000521 | 0.198 | RNF26 | ring finger protein 26 |
| 2703 | 9.34 | 7.91 | −1.43 | −6.43 | 0.00301 | 0.248 | GJA8 | gap junction protein; alpha 8; 50 kDa |
| *8086148 | 9.88 | 8.46 | −1.42 | −14.9 | 0.000118 | 0.189 | NA | NA |
| 221914 | 7.55 | 8.95 | 1.4 | 6.42 | 0.00303 | 0.248 | GPC2 | glypican 2 |
| 183 | 11 | 9.63 | −1.37 | −6.96 | 0.00224 | 0.235 | AGT | angiotensinogen (serpin peptidase |
| | | | | | | | | inhibitor; clade A; member 8) |
| 26052 | 4.04 | 2.7 | −1.34 | −6.4 | 0.00306 | 0.248 | DNM3 | dynamin 3 |
| 644083 | 12.2 | 10.9 | −1.3 | −6.93 | 0.00228 | 0.235 | LOC644083 | hypothetical LOC644083 |
| *8133215 | 6.77 | 5.48 | −1.29 | −13.3 | 0.000185 | 0.198 | NA | NA |
| 27341 | 6.51 | 7.79 | 1.28 | 10.1 | 0.000541 | 0.198 | RRP7A | ribosomal RNA processing 7 |
| | | | | | | | | homolog A (S. cerevisiae) |
| 63926 | 9.03 | 10.3 | 1.27 | 6.83 | 0.0024 | 0.24 | ANKRD5 | ankyrin repeat domain 5 |
| *8102619 | 9.07 | 10.3 | 1.23 | 7.52 | 0.00167 | 0.223 | NA | NA |
| 10223 | 9.19 | 10.4 | 1.21 | 12.8 | 0.000215 | 0.198 | GPA33 | glycoprotein A33 (transmembrane) |
| 100129455 | 9.14 | 7.93 | −1.21 | −8.65 | 0.000983 | 0.204 | LOC100129455 | hypothetical LOC100129455 |
| 23623 | 9.7 | 10.9 | 1.2 | 7.38 | 0.0018 | 0.226 | RUSC1 | RUN and SH3 domain containing 1 |
| 8872 | 8.78 | 9.98 | 1.2 | 7.12 | 0.00206 | 0.232 | CDC123 | cell division cycle 123 homolog |
| | | | | | | | | (S. cerevisiae) |
| *8164100 | 11.7 | 10.5 | −1.2 | −10.4 | 0.000483 | 0.198 | NA | NA |
| 387695 | 8.38 | 7.19 | −1.19 | −10.3 | 0.000501 | 0.198 | C10orf99 | chromosome 10 open reading frame |
| | | | | | | | | 99 |
| 7433 | 4.28 | 3.09 | −1.19 | −7.2 | 0.00197 | 0.232 | VIPR1 | vasoactive intestinal peptide |
| | | | | | | | | receptor 1 |
| 55747 | 9.82 | 11 | 1.18 | 10.7 | 0.000432 | 0.198 | FAM21B | family with sequence similarity 21; |
| | | | | | | | | member B |
| *8125835 | 10.9 | 9.72 | −1.18 | −10.1 | 0.000541 | 0.198 | NA | NA |
| 9219 | 8.87 | 7.7 | −1.17 | −7.89 | 0.0014 | 0.216 | MTA2 | metastasis associated 1 family; |
| | | | | | | | | member 2 |
| *8084945 | 8.6 | 7.44 | −1.16 | −10.6 | 0.000448 | 0.198 | NA | NA |
| *8180355 | 10.7 | 9.55 | −1.15 | −10.2 | 0.000521 | 0.198 | NA | NA |
| 149041 | 9.56 | 10.7 | 1.14 | 7.31 | 0.00186 | 0.229 | RC3H1 | ring finger and CCCH-type zinc |
| | | | | | | | | finger domains 1 |
| 23065 | 6.16 | 7.28 | 1.12 | 7.48 | 0.00171 | 0.223 | KIAA0090 | KIAA0090 |
| 8894 | 10.7 | 9.58 | −1.12 | −11.1 | 0.000375 | 0.198 | EIF2S2 | eukaryotic translation initiation |
| | | | | | | | | factor 2; subunit 2 beta; 38 kDa |
| *8163629 | 8.87 | 7.75 | −1.12 | −7.95 | 0.00136 | 0.216 | NA | NA |
| 79929 | 5.72 | 4.61 | −1.11 | −11.7 | 0.000305 | 0.198 | MAP6D1 | MAP6 domain containing 1 |
| 391196 | 11.9 | 10.8 | −1.1 | −7.64 | 0.00158 | 0.22 | OR2M7 | olfactory receptor; family 2; |
| | | | | | | | | subfamily M; member 7 |
| 728118 | 10.2 | 11.3 | 1.1 | 6.54 | 0.00282 | 0.248 | FAM22A | family with sequence similarity 22; |
| | | | | | | | | member A |
| *8178811 | 8.6 | 9.7 | 1.1 | 7.18 | 0.00199 | 0.232 | NA | NA |
| 53836 | 11.6 | 10.5 | −1.1 | −7.56 | 0.00164 | 0.222 | GPR87 | G protein-coupled receptor 87 |
| *8180003 | 8.48 | 9.58 | 1.1 | 7.22 | 0.00195 | 0.232 | NA | NA |
| 55720 | 11.7 | 10.6 | −1.1 | −7.02 | 0.00217 | 0.234 | TSR1 | TSR1; 20S rRNA accumulation; |
| | | | | | | | | homolog (S. cerevisiae) |
| 8756 | 5.69 | 6.75 | 1.06 | 6.45 | 0.00297 | 0.248 | ADAM7 | ADAM metallopeptidase domain 7 |
| 3735 | 6.15 | 7.21 | 1.06 | 6.68 | 0.00261 | 0.246 | KARS | lysyl-tRNA synthetase |
| 339390 | 8.12 | 7.1 | −1.02 | −7.35 | 0.00182 | 0.227 | CLEC4G | C-type lectin superfamily 4; member G |
| 5314 | 7.5 | 6.49 | −1.01 | −9.22 | 0.000769 | 0.2 | PKHD1 | polycystic kidney and hepatic |
| | | | | | | | | disease 1 (autosomal recessive) |
| *8118824 | 7.64 | 6.63 | −1.01 | −7.59 | 0.00162 | 0.222 | NA | NA |

*ProbeSet ID number; refers to the identity of the probe used in the Affymetric human Gene 1.0 ST array.

Genes, which qualified in the stringent statistical tests, were used for gene ontology and pathway analysis. Expression data sets containing gene identifier and their corresponding expression values, as fold-changes, were uploaded as a tab-delimited text file to the Ingenuity pathway Analysis (IPA) software (Ingenuity systems, Mountain view, Calif.). Genes, which mapped to the ingenuity pathway database, were categorized based on molecular functions, gene ontology and biological processes. Each class was grouped based on their p-value. The identified genes named as focused genes were also mapped to genetic networks in the IPA database and ranked by score. The calculated probability score represented whether a collection of genes in a network could be found by chance alone.

### MASS Algorithm

Microarray Analysis Suite 5.0 (MAS 5.0) algorithm was developed by Affymetrix to measure the relative intensities from microarray experiments using the Affymetrix GeneChip arrays. The signal is calculated using the One-Step Tukey's Biweight Estimate, which yields a robust weighted mean that is relatively insensitive to outliers. The Tukey's Biweight method gives an estimate of the amount of variation in the data, exactly as standard deviation measures the amount of variation for an average. MAS 5.0 subtracts a “stray signal” estimate from the PM signal that is based on the intensity of the MM signal. However, in cases where the MM signal outweighs the PM signal, an adjusted value is used. These adjustments will eliminate negative values.

### RMA Algorithm

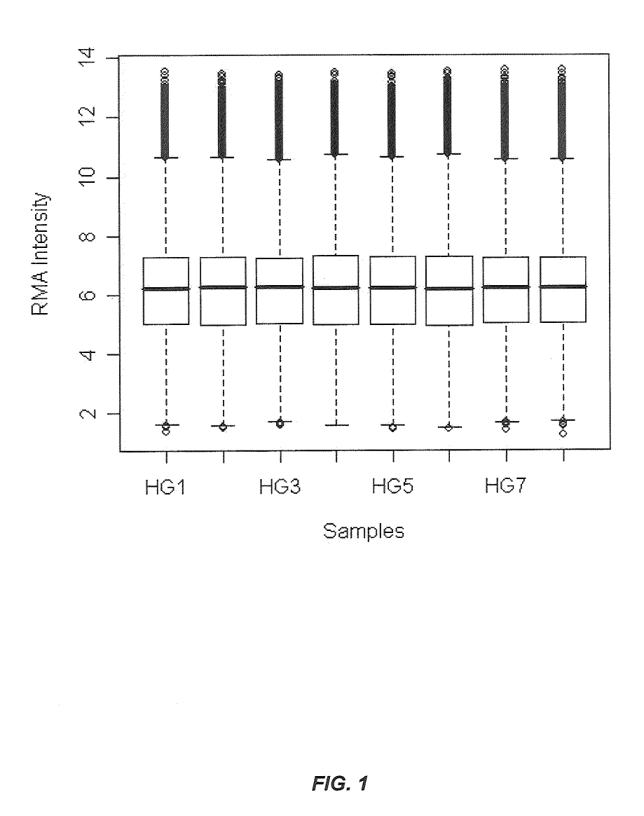

The data from the eight microarrays was also pre-processed using the RMA Algorithm (Irizarry, R A et al., Biostatistics, 4, 249-264 (2003)). The output of the algorithm is raw gene expression intensities expressed in log2 scale. A plot of the intensities of all of the samples is shown in FIG. 1.

### ANOVA Test

Analysis of variance (ANOVA) is a collection of statistical models, and their associated procedures, in which the observed variance is partitioned into components due to different explanatory variables. ANOVA is commonly used to compare the means of more than 2 groups.

An analysis of variance (ANOVA) was preformed on each probe set. The test is designed to detect differentially expressed genes (DEGs) between any pair of groups. The p-values were adjusted to control the false discovery rate (FDR) in multiple hypothesis tests (Benjamini & Hochberg, 1995). Table 4 shows a number of DEGs at various raw p-value and FDR-adjusted p-value thresholds. Using a threshold of p.fdr <0.25, there are 228 differentially expressed genes (DEGs) cumulatively. The gene expression levels of the top 5 DEGs are plotted in FIG. 2.

### Multiple Hypothesis Test

In a hypothesis test, an acceptable maximum probability of rejecting the null hypothesis when it is true, thus committing a Type I error, is typically specified. In a microarray study, a large number of hypothesis tests are performed. When many hypotheses are tested, and each test has a specified Type I error probability, the probability that at least some Type I errors are committed increase, often sharply, with the number of hypotheses. To control the overall Type I error, an adjustment on statistical test p-values is applied to control the overall false discovery rate or FDR (Benjamini & Hochberg, 1995). Other available multiple hypothesis correction methods include Bonferroni correction in which the p-values are multiplied by the number of comparisons, Holm correction (Holm, 1979), Hochberg correction (Hochberg, 1988), and Hommel correction (Hommel, 1988).

### Hierarchical Clustering Analysis

Hierarchical clustering analysis is a statistical method for finding relatively homogeneous clusters of cases based on measured characteristics. It starts with each case in a separate cluster and then combines the clusters sequentially, reducing the number of clusters at each step until only one cluster is left. When there are N cases, this involves N−1 clustering steps, or fusions. A heatmap with two dimension hierarchical clustering results are frequently used in the microarray analysis to demonstrate the sample and gene clustering structure based on gene expression profiles.

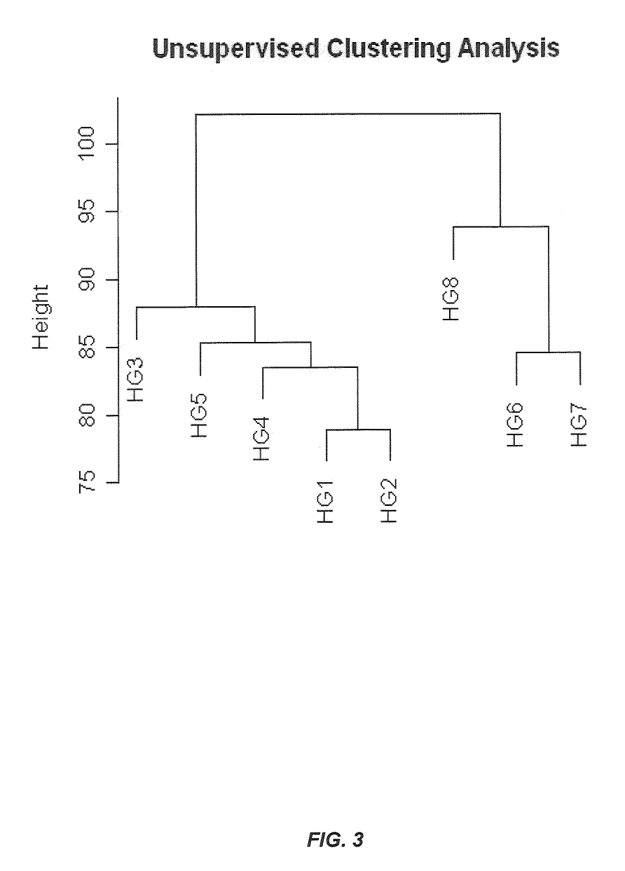

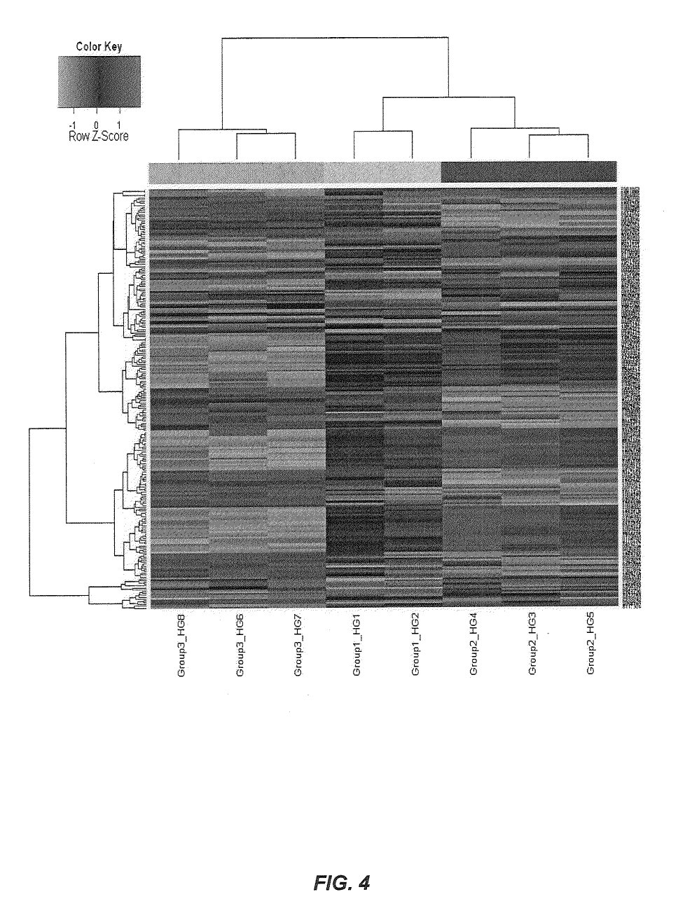

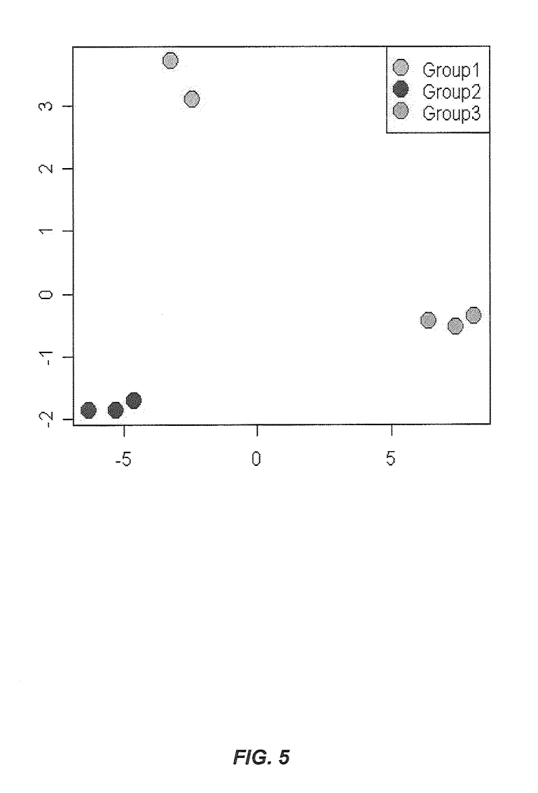

Hierarchical clustering analysis was performed to explore whether the gene expression profiles of the DEGs can separate the IBS and control samples into distinct classes. All unmasked probe sets were used in this analysis. FIG. 3 shows the clustering results and FIG. 4 provides a heatmap illustrating the differential expression of a set of genes the include the selected 72-gene subset shown in Table 4. The IBS-C (group 1; HG1 and 2), IBS-D (group 2; HG3, 4, and 5), and control (group 3; HG6, 7, and 8) groups are completely separated by the gene expression profiles of the DGEs, which are indicated by the color panel on the top of the heatmap.

### Multidimensional Scaling

Multidimensional scaling (MDS) is a set of related statistical techniques often used in information visualization for exploring similarities or dissimilarities in data. MDS is a special case of ordination. An MDS algorithm starts with a matrix of item—item similarities, then assigns a location of each item in a low-dimensional space, suitable for 2D or 3D visualization. A plot of the separation among samples based on the gene expression profiles of all unmasked probe sets is shown in FIG. 5.

### Principal Component Analysis

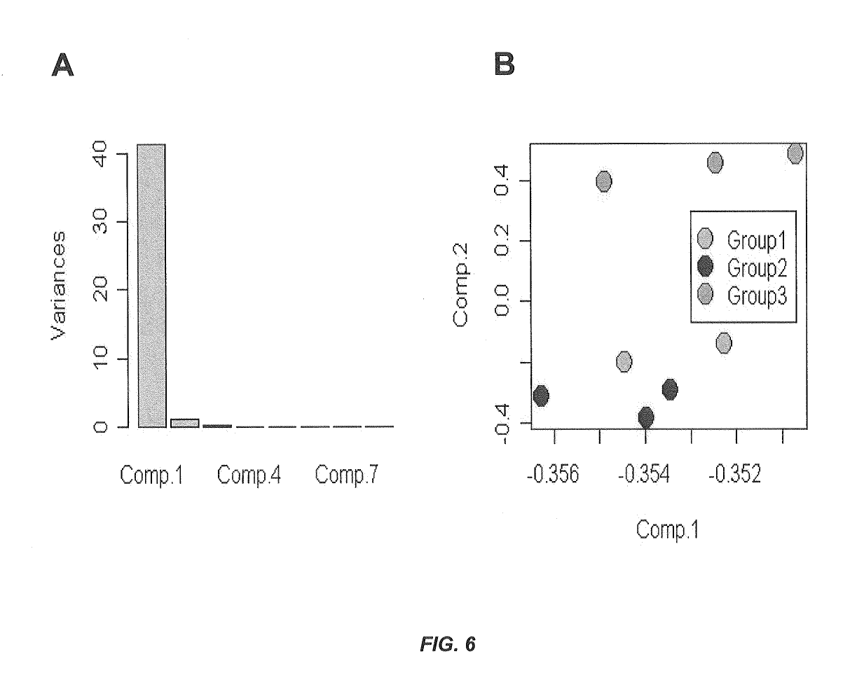

Principal component analysis (PCA) involves a mathematical procedure that transforms a number of (possibly) correlated variables into a (smaller) number of uncorrelated variables called principal components. The first principal component accounts for as much of the variability in the data as possible, and each succeeding component accounts for as much of the remaining variability as possible. FIG. 6A illustrates the variation that can be explained by each of the top principal components. FIG. 6B illustrates the separation of the samples by the top 2 principal components.

T-Test

The t-test assesses whether the means of two groups are statistically different from each other. This analysis allows for comparison of the means of two groups. A pair-wise t-test was performed between each pair of groups. Fold change, p-value and FDR-adjusted p-value (Benjamini & Hochberg, 1995) were computed for each probe set on the array in each comparison. Differentially expressed genes (DEGs) were defined as those genes that have a FDR-adjusted p-value <0.25 and a 2 log2 fold change >2. For example, Table 4 shows 72 DEGs between Group 2 and Group 3 ordered by fold change.

### Volcano Plot

Volcano plot arranges genes along dimensions of biological and statistical significance. The first (horizontal) dimension is the fold change between the two groups (on a log scale, so that up and down regulation appear symmetric), and the second (vertical) axis represents the p-value for a t-test of differences between samples (most conveniently on a negative log scale, such that smaller p-values appear higher up). The first axis indicates biological impact of the change; the second indicates the statistical evidence, or reliability of the change.

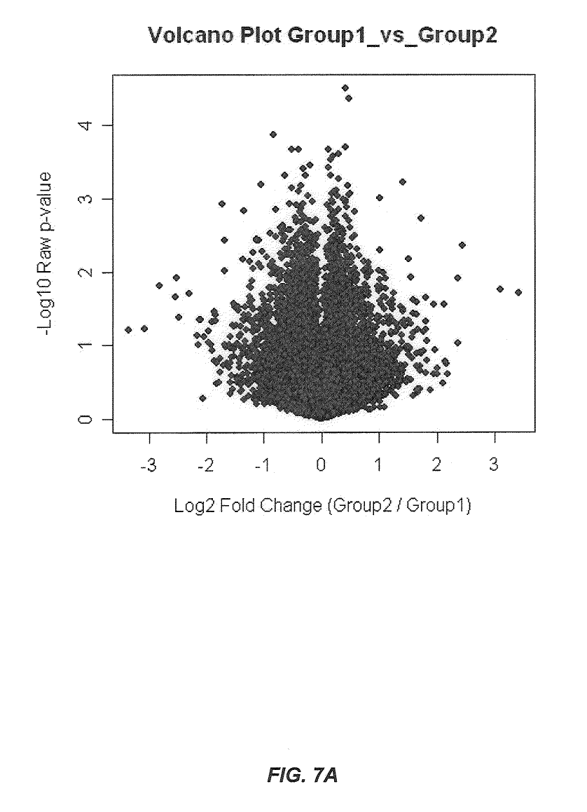

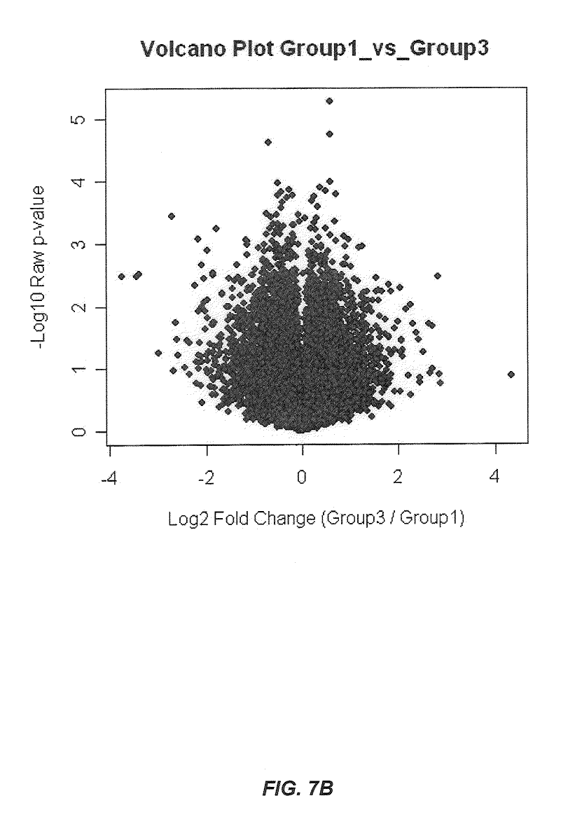

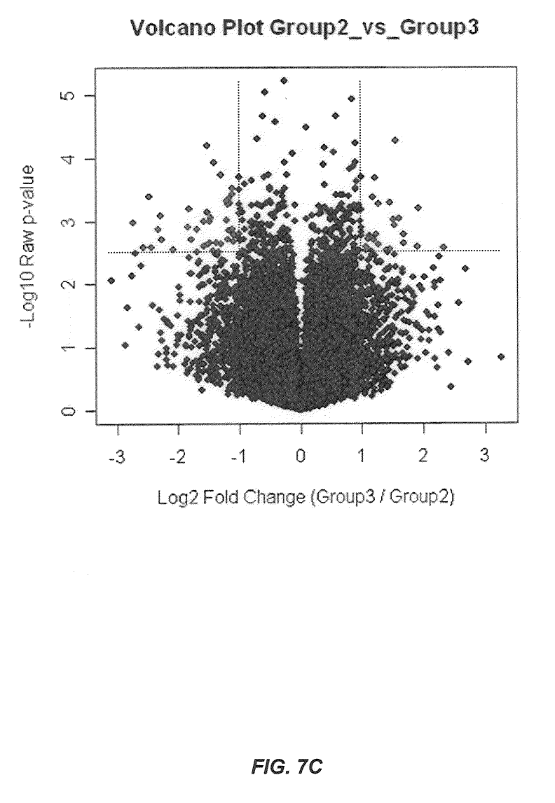

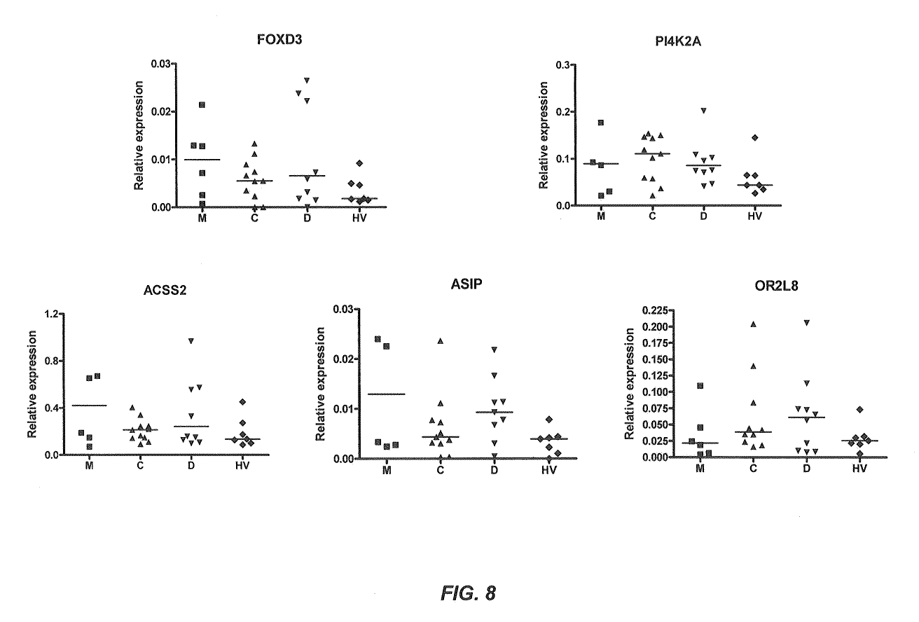

Volcano plots show the relationship between biological significance (fold change) and statistical significance (p-value). FIGS. 7A-C show volcano plots of the comparison between each pair of groups ((A) IBS-C vs IBS-D groups, (B) IBS-C vs control groups, and (C) IBS-D vs control groups). Although the raw p-values are plotted on Y-axis, DEGs were determined by a threshold of FDR-adjusted p-value <0.25 and fold change >2, the boundaries of which are marked with a dashed line in FIG. 7C. DEGs are highlighted in red color.

### Fisher Exact Test

The Fisher exact test is a statistical test used to determine if there are nonrandom associations between two categorical variables. The Fisher Exact Test looks at a contingency table which displays how the first variable affects the second variable or in reverse. Its null hypothesis is that the two are independent.

### C. Example 3

In order to verify the utility of the IBS markers identified by the microarray experiments, the mRNA expression levels of five identified genes were validated using quantitative reverse transcript-polymerase chain reaction (qRT-PCR) analysis. Briefly, cDNA was synthesized from RNA samples from 12 IBS-M, 22 IBS-C, 12 IBS-D, and 21 control subjects by PCR RNA core kit (Applied Biosystems, Bedford, Mass.). Real time quantitative reverse transcript-polymerase chain reaction (qRT-PCR) with SYBER Green, using gene-specific PCR primers, was performed to verify the microarray data. Five genes (FOXD3, PI4K2A, ACSS2, ASIP, and OR2L8), having Log2 fold changes >2 and FDR adjusted p-values <0.25, were selected and primers used to amplify each gene were generated. Samples were run in triplicate, and PCR was performed by an ABI 7700 thermocycler (Applied Biosystems, Bedford, Mass.). Results of the qRT-PCR analysis is shown in FIG. 8.

### D. Example 4

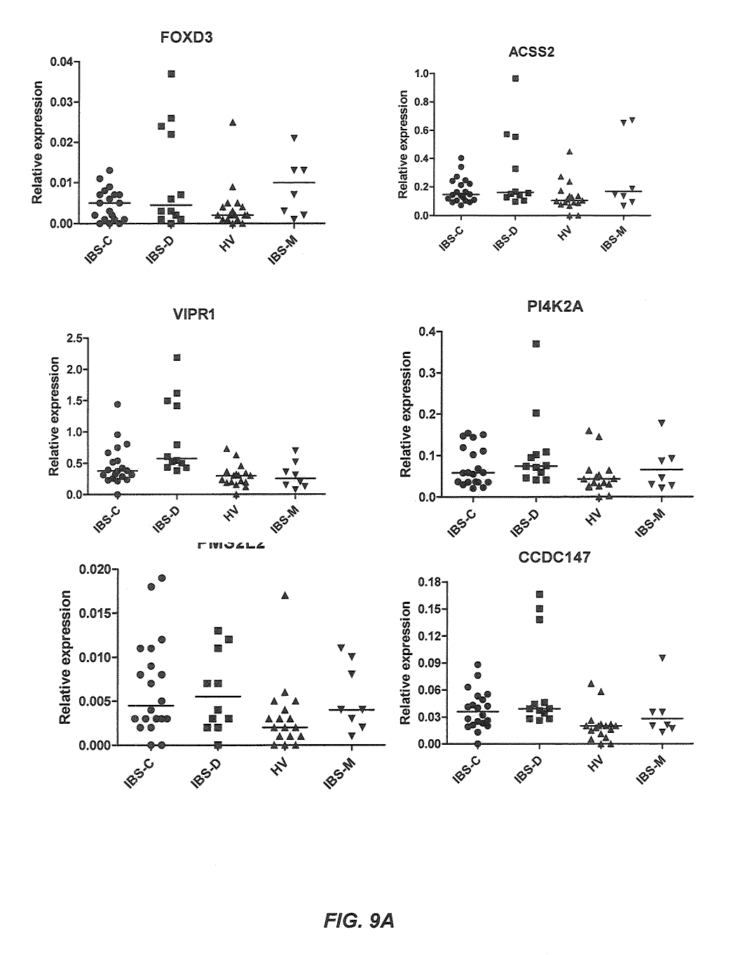

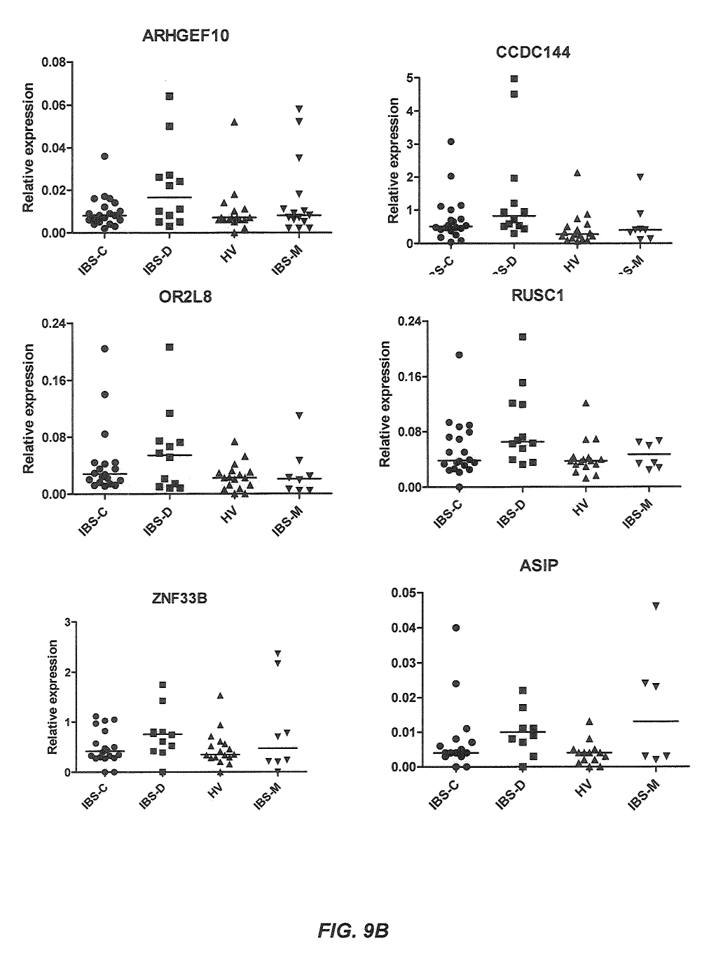

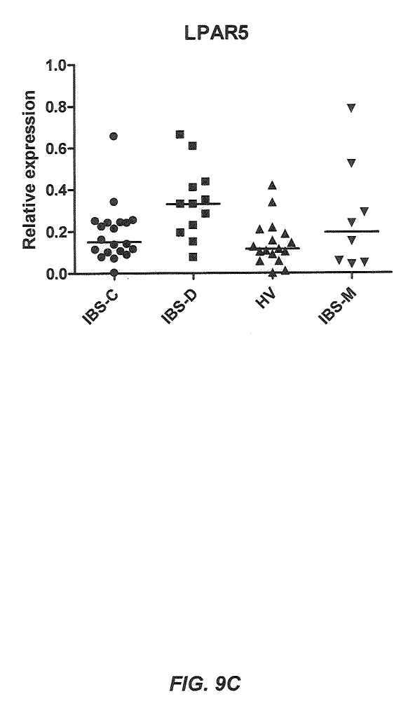

To further validate the utility of the novel gene expression IBS markers identified above, the expression levels of 14 of the top 72 discovery phase genes, as determined by Log2-fold change, were assayed in a clinical study of independently ascertained, consecutively enrolled, prospective cohorts of 98 patients with IBS. Each patient was diagnosed by a board-certified gastroenterologist; IBS was confirmed by biopsy and IBS met Rome III criteria. All protocols were IRB approved; informed consent was obtained and peripheral blood samples and clinical data were collected from all patients. Expression data was obtained from peripheral whole blood samples by isolating total mRNAs, synthesizing cDNAs, and performing real-time quantitative PCR. Expression levels of the candidate biomarker genes were assayed on each patient specimen and normalized to a within-patient reference gene. The expression levels of the selected biomarkers is shown in FIGS. 9A-C.

cDNA was synthesized from RNA samples by PCR RNA core kit (Applied Biosystems, Bedford, Mass.). Real time quantitative reverse transcript-polymerase chain reaction (qRT-PCR) with SYBER Green, using gene-specific PCR primers, was performed to verify the microarray data. Samples were run in triplicate, and PCR was performed by an ABI 7700 thermocycler (Applied Biosystems, Bedford, Mass.). Expression of the house keeping gene β-actin was determined for normalization, following the geNorm method. A linear regression analysis was performed and the coefficient of variation was calculated to assess a correlation between the RT-PCR and gene array results of these selected genes. Log2-fold changes and p-values for the expression of the candidate genes is shown in Table 5.

TABLE 5
Log2-fold changes and calculated p-values for the 14 selected
candidate IBS marker genes validated by qRT-PCR.

| | | P |
|---|---|---|
| GENE | SCORE | VALUE |
| CCDC147 | 6.425 | 0.011 |
| VIPR1 | 4.818 | 0.028 |
| LPAR5 | 3.881 | 0.049 |
| CCDC144A | 2.944 | 0.086 |
| GNG3 | 2.901 | 0.089 |
| ACSS2 | 2.516 | 0.113 |
| ZNF33B | 1.902 | 0.168 |
| PMS2L2 | 1.629 | 0.202 |
| RUSC1 | 1.384 | 0.239 |
| ARHGE | 1.226 | 0.268 |
| ASIP | 1.119 | 0.29 |
| OR2L8 | 1.094 | 0.296 |
| PI4K2A | 0.578 | 0.447 |
| FOXD3 | 0.553 | 0.457 |

### E. Example 5

In order to further determine the gene expression patterns most predictive of IBS, the raw gene expression data obtained in Example 2 was analyzed by using analysis of variance (ANOVA) to compare the means of hybridization signals in all three groups. IBS-D and healthy volunteer groups were compared using t-test. Genes that are statistically different in the two groups were assesed. The analysis software used are Affymetrix Command Console, Affymetrix Expression Console, and R.

### Network, Gene Ontology and Canonical Pathways Analysis

Genes, which qualified in the stringent statistical tests, were used for gene ontology and pathway analysis. Expression data sets containing gene identifier and their corresponding expression values, as fold-changes, were uploaded as a tab-delimited text file to the Ingenuity pathway Analysis (IPA) software (Ingenuity systems, Mountain view, Calif.). Genes, which mapped to the ingenuity pathway database, were categorized based on molecular functions, gene ontology and biological processes. Each class was grouped based on their p-value. The identified genes named as focused genes were also mapped to genetic networks in the IPA database and ranked by score. The calculated probability score represented whether a collection of genes in a network could be found by chance alone.

mRNA Expression Assay by Quantitative Reverse Transcript-Polymerase Chain Reaction (qRT-PCR)

66 selected genes were further validated by qRT-PCR. cDNA was synthesized from RNA samples by PCR RNA core kit (Applied Biosystems, Bedford, Mass.). Real time quantitative reverse transcript-polymerase chain reaction (qRT-PCR) with SYBER Green, using gene-specific PCR primers, was performed to verify the microarray data. Eleven genes were selected randomly and the primers used to amplify each gene are listed in the Additional File-1. Samples were run in triplicate, and PCR was performed by an ABI 7700 thermocycler (Applied Biosystems, Bedford, Mass.). Expression of multiple house keeping genes (GNB, β-actin, GAPDH and tubulin) were simultaneously determined for normalization, following the geNorm method. A linear regression analysis was performed and the coefficient of variation was calculated to assess a correlation between the RT-PCR and gene array results of these 11 randomly selected genes.

Identification of Differential Expressed Genes from the Affymetrix Chip Study.

Briefly, an analysis of variance (ANOVA) was performed on each probe set. The test is designed to detect differentially expressed genes between any pair of groups. The p-values were adjusted to control the false discovery rate (FDR) in multiple hypothesis tests (Benjamini & Hochberg, 1995). Using a threshold of p.fdr <0.25, 228 differentially expressed genes (DEGs) were identified cumulatively. A hierarchical clustering analysis was then performed to explore whether the gene expression profiles of the DEGs can separate samples into distinct classes. All unmasked probe sets were used in this analysis. FIG. 4 shows the clustering results. Three groups are completely separated by the gene expression profiles of the DGEs, which indicated by the color panel on the top of the heatmap (FIG. 4). The separation among samples was further visualized based on the gene expression profiles of all unmasked probe sets using a multidimensional scaling plot. (FIG. 5).

In order to select differentially expressed genes, a pair-wise t-test was performed between each pair of groups. Fold change, p value and FDR-adjusted p-value (Benjamini & Hochberg, 1995) were computed for each probe set on the array in each comparison. Differentially expressed genes (DEGs) were defined as those genes that have a FDR-adjusted p-value <0.25 and a fold change >2. For example, Table 6 shows 40 DEGs between IBS-D and healthy volunteers ordered by fold change. Complete list oft-test DEGs between each pair of groups are conducted. In order to identify genes which can be used for both IBS-C and IBS-D subgroup diagnosis, selected 26 genes were further selected which are up regulated in both groups based on fold changes and P values (Table 7).

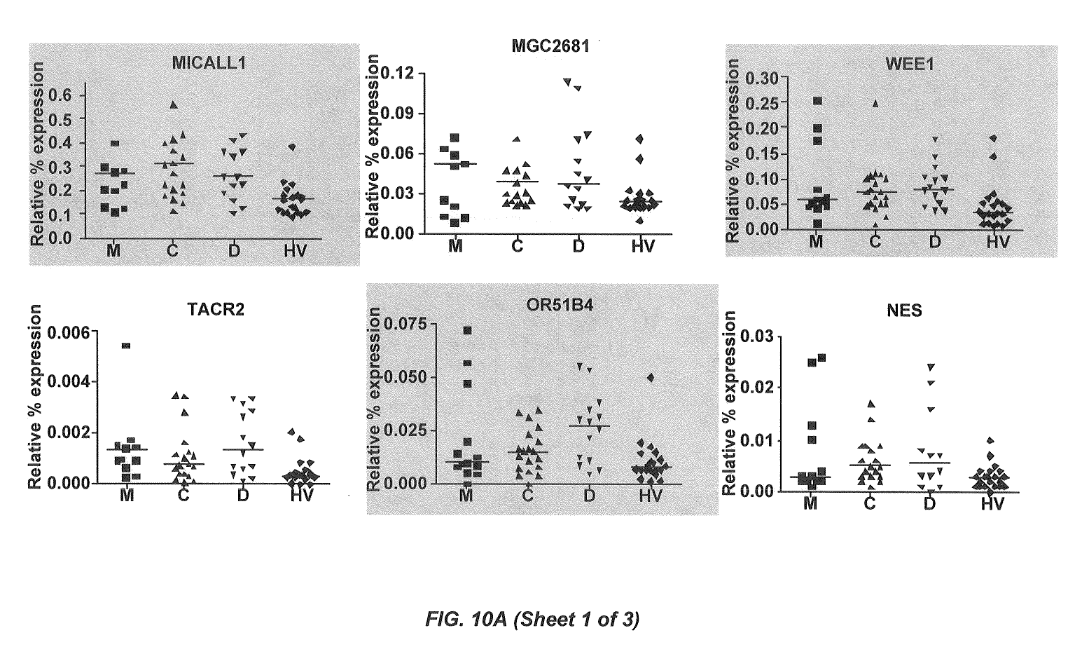

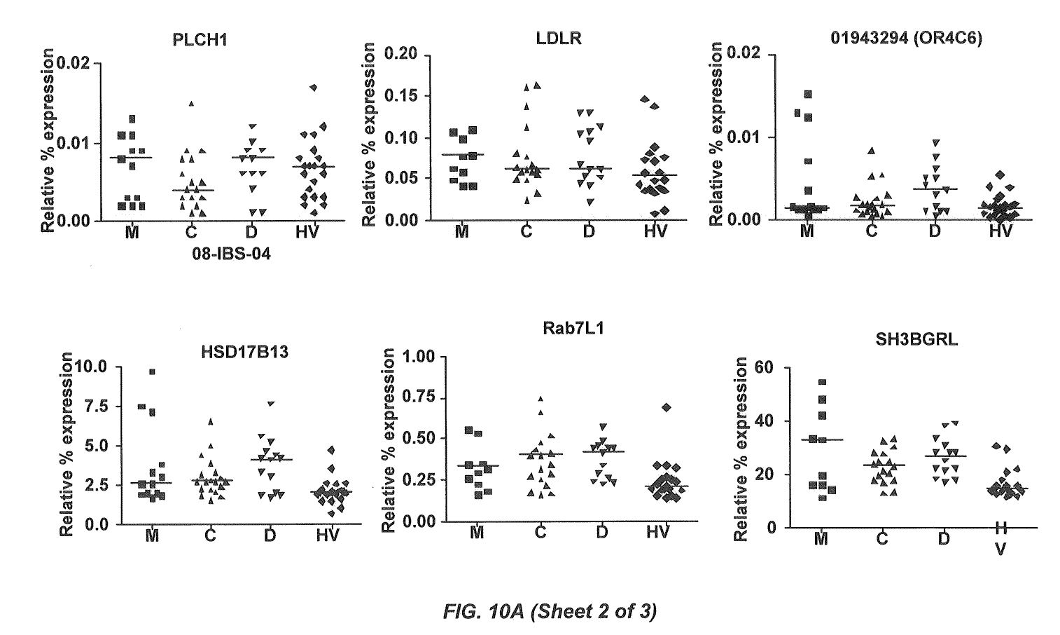

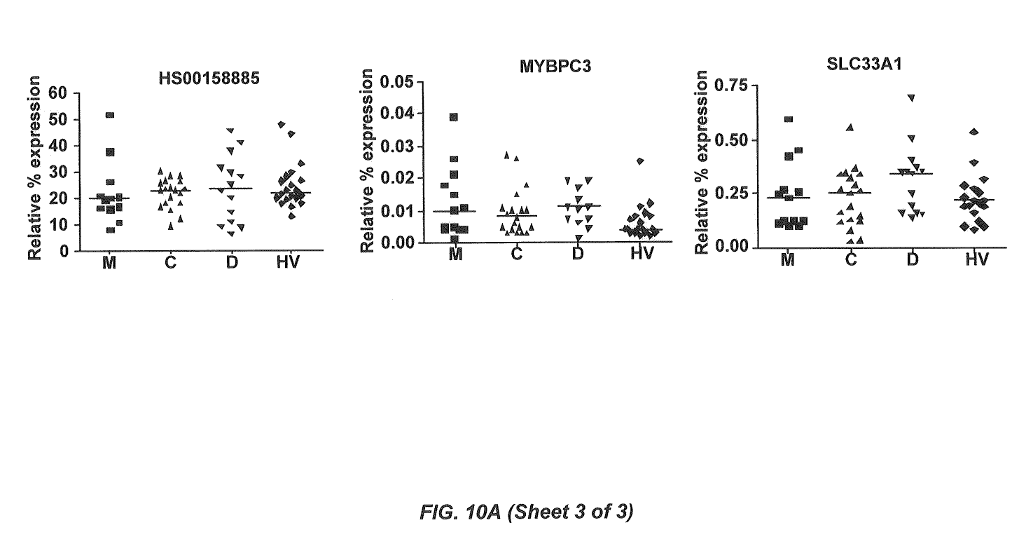

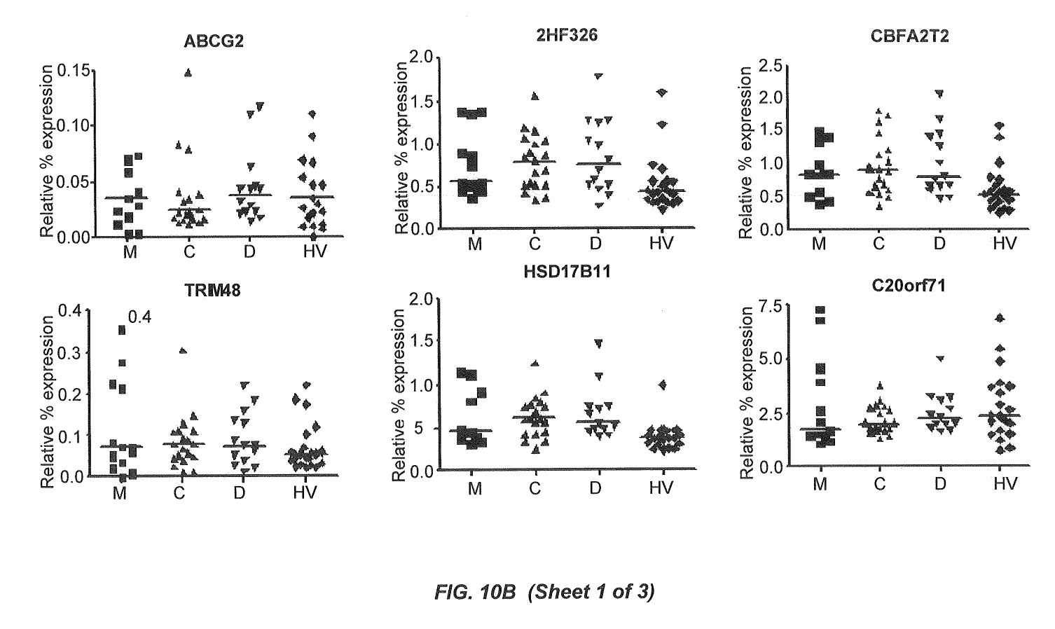

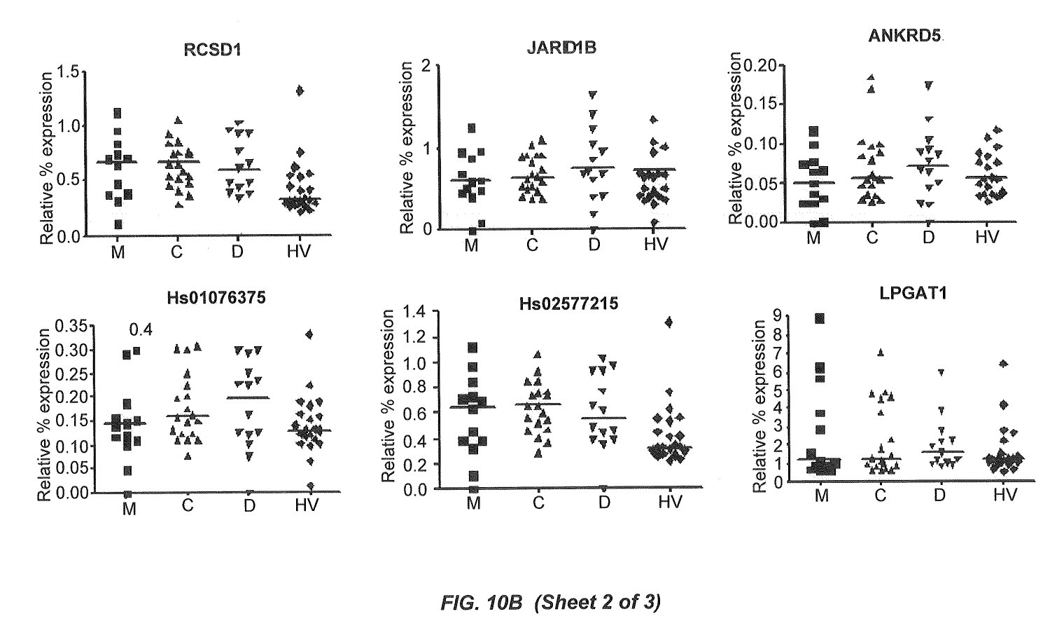

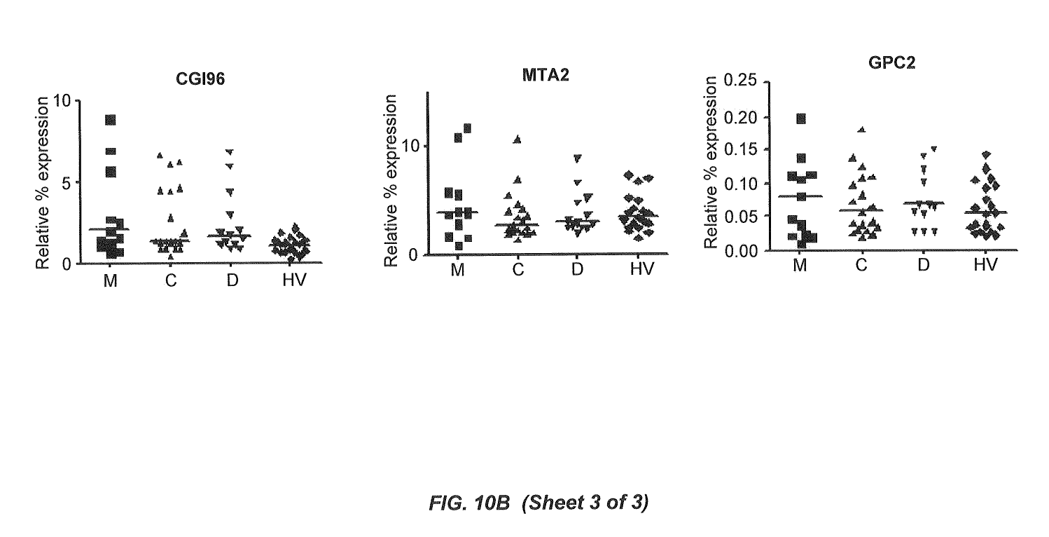

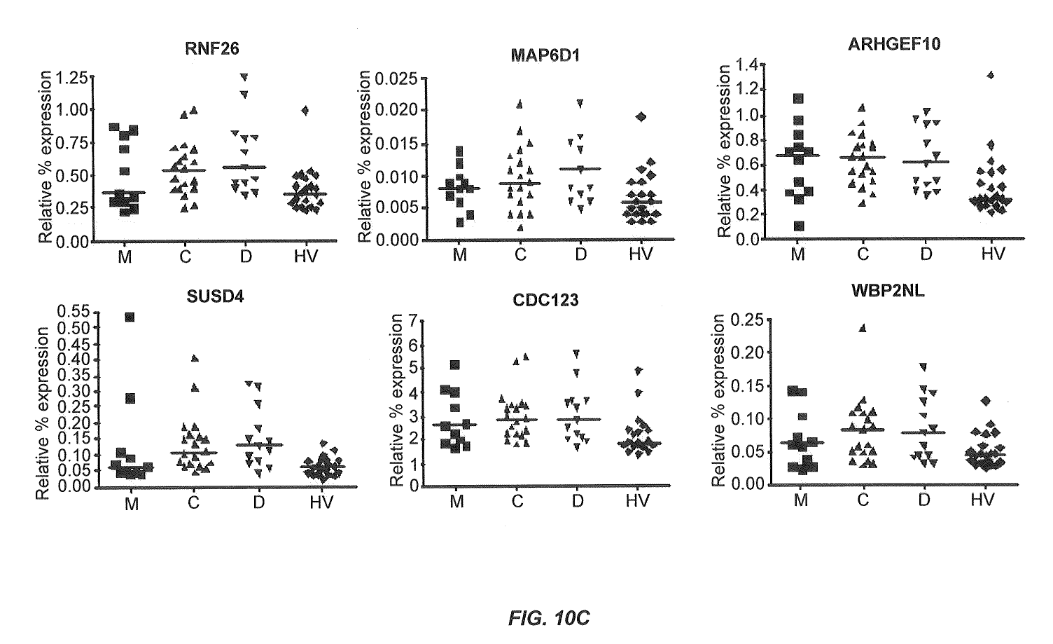

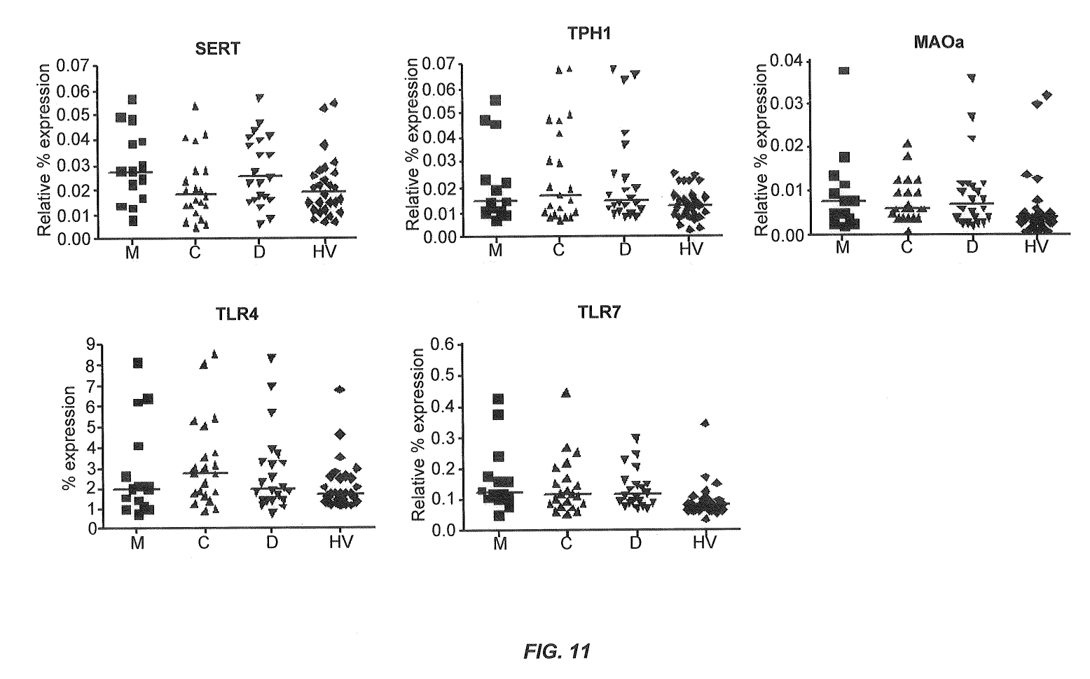

Real time quantitative PCR validation of selected differently expressed genes (DEGs). The 66 selected genes that identified from the microarray data analysis were further validated by q-PCR using the probes purchased from Applied Biosystems (Table 8). Relative expression of the selected genes were measured by standardizing expression of each gene with beta-actin in Paxgene blood samples from 27 healthy volunteers, 19 IBS-C, 22 IBS-D, and 17 IBS-M patients. Among the 66 selected genes, 16 genes were not detectable by RT q-PCR. The relative expression in the remaining 50 genes largely confirmed the microarray results with reference to fold change levels of individual IBS patients. Data for 36 of these q-PCR reactions is shown in FIG. 10. In addition, qRT-PCR data was also obtained for 5 targeted genes (SERT, TPH1, MAO-A, TLR2, and TLR4), which were not differentially expressed in IBS-C, IBS-D, and IBS-M patients (FIG. 11).

TABLE 6
Top 40 genes that differentially expressed in IBS-D vs. healthy volunteers.

| | | | Fold | t | | |
|---|---|---|---|---|---|---|
| Gene | IBS-D | HV | Change | statistic | P value | Gene Descriptions |
| CCDC147 | 5.39 | 7.32 | 6.8895 | 9.63 | 0.0007 | coiled-coil domain containing 147 |
| PI4K2A | 6.63 | 4.06 | 13.066 | −6.63 | 0.0027 | phosphatidylinositol 4-kinase type 2 alpha |
| ACSS2 | 8.92 | 6.44 | 11.941 | −10.8 | 0.0004 | acyl-CoA synthetase short-chain family member 2 |
| ASIP | 6.65 | 4.22 | 11.359 | −6.66 | 0.0026 | agouti signaling protein; nonagouti homolog (mouse) |
| OR2L8 | 6.13 | 8.47 | 10.381 | 6.56 | 0.0028 | olfactory receptor; family 2; subfamily L; member 8 |
| LPAR5 | 9.07 | 6.74 | 10.278 | −7.9 | 0.0014 | lysophosphatidic acid receptor 5 |
| JARID1B | 5.48 | 3.19 | 9.8749 | −9.01 | 0.0008 | jumonji; AT rich interactive domain 1B |
| CDKN1C | 6.34 | 4.07 | 9.6794 | −7.21 | 0.0020 | cyclin-dependent kinase inhibitor 1C (p57; Kip2) |
| MAP1LC3A | 8.92 | 6.44 | 11.941 | −10.8 | 0.0004 | microtubule-associated protein 1 light chain 3 alpha |
| FOXD3 | 6.71 | 3.98 | 15.333 | −8.51 | 0.0011 | forkhead box D3 |
| MAP1LC3A | 8.92 | 6.44 | 11.941 | −10.8 | 0.0004 | microtubule-associated protein 1 light chain 3 alpha |
| PRAMEF7 | 8.06 | 6.22 | 6.2965 | −8.15 | 0.0012 | PRAME family member 7 |
| GNG3 | 11 | 9.17 | 6.2339 | −9.66 | 0.0006 | guanine nucleotide binding protein (G protein); g3 |
| ARHGEF10L | 9.78 | 8 | 5.9299 | −7.16 | 0.0020 | Rho guanine nucleotide exchange factor (GEF) 10L |
| MAP1LC3C | 10.4 | 8.71 | 5.4195 | −7.98 | 0.0013 | microtubule-associated protein 1 light chain 3 |
| | | | | | | gamma |
| C20orf71 | 6.38 | 4.69 | 5.4195 | −6.4 | 0.0031 | chromosome 20 open reading frame 71 |
| C20orf114 | 6.38 | 4.69 | 5.4195 | −6.4 | 0.0031 | chromosome 20 open reading frame 114 |
| SUSD4 | 5.82 | 7.5 | 5.3656 | 6.91 | 0.0023 | sushi domain containing 4 |
| ZNF33B | 7.23 | 8.91 | 5.3656 | 7.49 | 0.0017 | zinc finger protein 33B |
| OR10W1 | 2.35 | 3.96 | 5.0028 | 8.77 | 0.0009 | olfactory receptor; family 10; subfamily W; member 1 |
| RCSD1 | 10.9 | 9.32 | 4.855 | −8.54 | 0.0010 | RCSD domain containing 1 |
| CCDC144A | 9.27 | 7.69 | 4.855 | −6.75 | 0.0025 | coiled-coil domain containing 144A |
| PMS2L2 | 5.96 | 7.52 | 4.7588 | 18.1 | 0.0001 | postmeiotic segregation increased 2-like 2 |
| ATRNL1 | 10.9 | 9.37 | 4.6182 | −17.3 | 0.0001 | attractin-like 1 |
| OR51E1 | 7.43 | 8.96 | 4.6182 | 8.21 | 0.0012 | olfactory receptor; family 51; subfamily E; member 1 |
| OR6Y1 | 7.18 | 8.7 | 4.5722 | 8.65 | 0.0010 | olfactory receptor; family 6; subfamily Y; member 1 |
| IAPP | 4.98 | 3.47 | 4.5267 | −6.86 | 0.0024 | islet amyloid polypeptide |
| LRRC18 | 11.4 | 9.92 | 4.3929 | −8.56 | 0.0010 | leucine rich repeat containing 18 |
| SNORD77 | 10.8 | 9.33 | 4.3492 | −8.86 | 0.0009 | small nucleolar RNA; C/D box 77 |
| LPGAT1 | 8.37 | 9.83 | 4.306 | 6.51 | 0.0029 | lysophosphatidylglycerol acyltransferase 1 |
| RNF26 | 7.12 | 8.58 | 4.306 | 10.2 | 0.0005 | ring finger protein 26 |
| GJA8 | 9.34 | 7.91 | 4.1787 | −6.43 | 0.0030 | gap junction protein; alpha 8; 50 kDa |
| GPC2 | 7.55 | 8.95 | 4.0552 | 6.42 | 0.0030 | glypican 2 |
| AGT | 11 | 9.63 | 3.9354 | −6.96 | 0.0022 | angiotensinogen (serpin peptidase inhibitor) |
| DNM3 | 4.04 | 2.7 | 3.819 | −6.4 | 0.0031 | dynamin 3 |
| LOC644083 | 12.2 | 10.9 | 3.6693 | −6.93 | 0.0023 | hypothetical LOC644083 |
| RRP7A | 6.51 | 7.79 | 3.5966 | 10.1 | 0.0005 | ribosomal RNA processing 7 homolog A (S. cerevisiae) |
| ANKRD5 | 9.03 | 10.3 | 3.5609 | 6.83 | 0.0024 | ankyrin repeat domain 5 |
| GPA33 | 9.19 | 10.4 | 3.3535 | 12.8 | 0.0002 | glycoprotein A33 (transmembrane) |
| LOC100129455 | 9.14 | 7.93 | 3.3535 | −8.65 | 0.0010 | hypothetical LOC100129455 |
| RUSC1 | 9.7 | 10.9 | 3.3201 | 7.38 | 0.0018 | RUN and SH3 domain containing 1 |

TABLE 7
26 genes which are up regulated in both groups (IBS-C and IBS-D) based on fold
changes and P values.

| | | | | Log2 fold | | | | | | |
|---|---|---|---|---|---|---|---|---|---|---|
| | | | | change | | t statistic | | p-value | | |
| Gene | | | | IBS-C | IBS-D | IBS-C | IBS-D | IBS-C | IBS-D | |
| Symbol | IBS-C | IBS-D | HV | vs HV | vs HV | vs HV | vs HV | vs HV | vs HV | Gene Descriptions |
| LOC399898 | 9.79 | 10.6 | 7.91 | 6.55 | 14.73 | −4.59 | −6.32 | 0.019 | 0.003 | hypothetical gene supported |
| | | | | | | | | | | by AK128188 |
| C20orf70 | 7.67 | 7.64 | 5.81 | 6.42 | 6.23 | −8.94 | −6.06 | 0.003 | 0.004 | chromosome 20 open |
| | | | | | | | | | | reading frame 70 |
| CHRNB2 | 8.37 | 8.73 | 7.06 | 3.71 | 5.31 | −4.3 | −5.7 | 0.023 | 0.005 | cholinergic receptor; |
| | | | | | | | | | | nicotinic; beta 2 (neuronal) |
| OR51B4 | 8.13 | 8.91 | 6.3 | 6.23 | 13.6 | −4.71 | −5.57 | 0.018 | 0.005 | olfactory receptor; family |
| | | | | | | | | | | 51; subfamily B |
| ZNF326 | 11.1 | 11.2 | 9.64 | 4.31 | 4.76 | −5.08 | −5.4 | 0.015 | 0.006 | zinc finger protein 326 |
| CBFA2T2 | 6.85 | 6.47 | 4.68 | 8.76 | 5.99 | −13.7 | −5.14 | 0.001 | 0.007 | core-binding factor; runt |
| | | | | | | | | | | domain; alpha subunit 2 |
| TACR2 | 11.7 | 12 | 10.2 | 4.48 | 6.05 | −4.25 | −5.13 | 0.024 | 0.007 | tachykinin receptor 2 |
| OR4C6 | 8.23 | 8.19 | 6.22 | 7.46 | 7.17 | −4.29 | −4.09 | 0.023 | 0.015 | olfactory receptor; family 4; |
| | | | | | | | | | | subfamily C; member 6 |
| MYBPC3 | 7.98 | 7.87 | 6.44 | 4.66 | 4.18 | −4.88 | −3.67 | 0.017 | 0.021 | myosin binding protein C; |
| | | | | | | | | | | cardiac |
| SCGB1C1 | 6.42 | 5.05 | 3.81 | 13.6 | 3.46 | −3.69 | −3.19 | 0.035 | 0.033 | secretoglobin; family 1C; |
| | | | | | | | | | | member 1 |
| HSD17B11 | 8.17 | 8.29 | 6.79 | 3.97 | 4.48 | −4.97 | −3.07 | 0.016 | 0.037 | hydroxysteroid (17-beta) |
| | | | | | | | | | | dehydrogenase 11 |
| SLC33A1 | 8.17 | 8.29 | 6.79 | 3.97 | 4.48 | −4.97 | −3.07 | 0.016 | 0.037 | solute carrier family 33 |
| | | | | | | | | | | (acetyl-CoA transporter); |
| ABCG2 | 8.17 | 8.29 | 6.79 | 3.97 | 4.48 | −4.97 | −3.07 | 0.016 | 0.037 | ATP-binding cassette; sub- |
| | | | | | | | | | | family G (WHITE); |
| HSD17B13 | 8.17 | 8.29 | 6.79 | 3.97 | 4.48 | −4.97 | −3.07 | 0.016 | 0.037 | hydroxysteroid (17-beta) |
| | | | | | | | | | | dehydrogenase 13 |
| PLCH1 | 8.17 | 8.29 | 6.79 | 3.97 | 4.48 | −4.97 | −3.07 | 0.016 | 0.037 | phospholipase C; eta 1 |
| LSP1 | 11.1 | 11.2 | 9.52 | 4.85 | 5.37 | −2.79 | −3 | 0.068 | 0.04 | lymphocyte-specific protein 1 |
| MBL2 | 6.84 | 7.19 | 5.01 | 6.23 | 8.85 | −3.35 | −2.97 | 0.044 | 0.041 | mannose-binding lectin |
| | | | | | | | | | | (protein C) 2; soluble |
| CCDC65 | 10.5 | 9.59 | 8.26 | 9.39 | 3.78 | −7.64 | −2.95 | 0.005 | 0.042 | coiled-coil domain |
| | | | | | | | | | | containing 65 |
| NES | 6.8 | 6.12 | 4.64 | 8.67 | 4.39 | −5.48 | −2.94 | 0.012 | 0.042 | nestin |
| MICALL1 | 8.28 | 8.09 | 6.58 | 5.47 | 4.53 | −3.01 | −2.9 | 0.057 | 0.044 | MICAL-like 1 |
| WBP2NL | 9.7 | 9.23 | 8.03 | 5.31 | 3.32 | −6.87 | −2.75 | 0.006 | 0.051 | WBP2 N-terminal like |
| TRIM48 | 4.96 | 5.08 | 3.64 | 3.74 | 4.22 | −4.45 | −2.75 | 0.021 | 0.051 | tripartite motif-containing |
| | | | | | | | | | | 48 |
| SH3BGRL3 | 6.76 | 5.59 | 3.31 | 31.5 | 9.78 | −8.61 | −2.63 | 0.003 | 0.058 | SH3 domain binding |
| | | | | | | | | | | glutamic acid-rich protein |
| | | | | | | | | | | like 3 |
| LDLR | 9.23 | 9.04 | 7.35 | 6.55 | 5.42 | −3.04 | −2.63 | 0.056 | 0.058 | low density lipoprotein |
| | | | | | | | | | | receptor |
| RAB7L1 | 5.88 | 4.3 | 2.48 | 29.96 | 6.17 | −8.88 | −2.54 | 0.003 | 0.064 | RAB7; member RAS |
| | | | | | | | | | | oncogene family-like 1 |
| WEE1 | 6.69 | 4.87 | 3.71 | 19.69 | 3.19 | −3.01 | −2.36 | 0.057 | 0.078 | WEE1 homolog (S. pombe) |

TABLE 8
66 differentially expressed genes selected for qRT-PCR validation.

| Gene Symbol | Gene Name | Assay ID |
|---|---|---|
| FOXD3 | forkhead box D3 | Hs00255287_s1 |
| PI4K2A | phosphatidylinositol 4-kinase type 2 alpha | Hs00218300_m1 |
| ACSS2 | acyl-CoA synthetase short-chain family member 2 | Hs00218766_m1 |
| ASIP | agouti signaling protein, nonagouti homolog (mouse) | Hs00181770_m1 |
| OR2L8 | olfactory receptor, family 2, subfamily L, member 8 | Hs02338632_g1 |
| LPAR5 | lysophosphatidic acid receptor 5 | Hs01051307_m1 |
| JARID1B | jumonji, AT rich interactive domain 1B | Hs00981910_m1 |
| CDKN1C | cyclin-dependent kinase inhibitor 1C (p57, Kip2) | Hs00175938_m1 |
| CCDC147 | coiled-coil domain containing 147 | Hs01001247_m1 |
| GNG3 | guanine nucleotide binding protein (G protein), gamma 3 | Hs00360009_g1 |
| ARHGEF10 | Rho guanine nucleotide exchange factor (GEF) 10 | Hs00744267_s1 |
| C20orf71 | chromosome 20 open reading frame 71 | Hs00420455_m1 |
| C20orf114 | chromosome 20 open reading frame 114 | Hs01113243_m1 |
| SUSD4 | sushi domain containing 4 | Hs00215864_m1 |
| ZNF33B | zinc finger protein 33B | Hs00300609_s1 |
| OR10W1 | olfactory receptor, family 10, subfamily W, member 1 | Hs01398519_s1 |
| RCSD1 | RCSD domain containing 1 | Hs00364590_m1 |
| CCDC144A | coiled-coil domain containing 144 family | Hs00417617_m1 |
| PMS2L2 | postmeiotic segregation increased 2-like 2 pseudogene | Hs02379621_u1 |
| ATRNL1 | attractin-like 1 | Hs00390459_m1 |
| OR51E1 | olfactory receptor, family 51, subfamily E, member 1 | Hs00379183_m1 |
| IAPP | islet amyloid polypeptide | Hs00169095_m1 |
| LRRC18 | leucine rich repeat containing 18 | Hs00736427_m1 |
| SNORD77 | lysophosphatidylglycerol acyltransferase 1 | Hs00360353_m1 |
| RNF26 | ring finger protein 26 | Hs00259249_s1 |
| GJA8 | gap junction protein, alpha 8, 50 kDa | Hs01102028_m1 |
| GPC2 | glypican 2 | Hs00415099_m1 |
| AGT | angiotensinogen (serpin peptidase inhibitor) | Hs00174854_m1 |
| DNM3 | dynamin 3 | Hs00399015_m1 |
| RRP7A | ribosomal RNA processing 7 homolog A (S. cerevisiae) | Hs00414229_m1 |
| ANKRD5 | ankyrin repeat domain 5 | Hs00223080_m1 |
| GPA33 | glycoprotein A33 (transmembrane) | Hs00170690_m1 |
| RUSC1 | RUN and SH3 domain containing 1 | Hs00204904_m1 |
| CDC123 | cell division cycle 123 homolog (S. cerevisiae) | Hs00195709_m1 |
| VIPR1 | vasoactive intestinal peptide receptor 1 | Hs00270351_m1 |
| MTA2 | metastasis associated 1 family, member 2 | Hs00191018_m1 |
| RC3H1 | ring finger and CCCH-type zinc finger domains 1 | Hs02577215_m1 |
| KIAA0090 | KIAA0090 | Hs01076375_m1 |
| GPR87 | G protein-coupled receptor 87 | Hs00225057_m1 |
| MAP6D1 | MAP6 domain containing 1 | Hs00227533_m1 |
| LOC399898 | hypothetical gene supported by AK128188 | Hs02385591_s1 |
| c20orf70 | chromosome 20 open reading frame 70 | Hs00395980_m1 |
| CHRNB2 | cholinergic receptor, nicotinic, beta 2 (neuronal) | Hs00181267_m1 |
| OR51B4 | olfactory receptor, family 51, subfamily B, member 4 | Hs00264159_s1 |
| ZNF326 | zinc finger protein 326 | Hs00299025_m1 |
| CBFA2T2 | core-binding factor, runt domain, alpha subunit 2; | Hs00955778_m1 |
| | translocated to, 2 | |
| TACR2 | tachykinin receptor 2 | Hs00169052_m1 |
| OR4C6 | olfactory receptor, family 4, subfamily C, member 6 | Hs01943294_s1 |
| MYBPC3 | myosin binding protein C, cardiac | Hs00165232_m1 |
| SCGB1C1 | secretoglobin, family 1C, member 1; secretoglobin, | Hs00377337_m1 |
| | family 1C, member 1-like | |
| HSD17B11 | hydroxysteroid (17-beta) dehydrogenase 11 | Hs00212226_m1 |
| SLC33A1 | solute carrier family 33 (acetyl-CoA transporter), | Hs00270469_m1 |
| | member 1 | |
| ABCG2 | ATP-binding cassette, sub-family G (WHITE), member 2 | Hs01053790_m1 |
| HSD17B13 | hydroxysteroid (17-beta) dehydrogenase 13 | Hs00418210_m1 |
| PLCH1 | phospholipase C, eta 1 | Hs00324566_m1 |
| LSP1 | lymphocyte-specific protein 1 | Hs00158885_m1 |
| MBL2 | mannose-binding lectin (protein C) 2, soluble | Hs00175093_m1 |
| CCDC65 | coiled-coil domain containing 65 | Hs00276995_m1 |
| NES | nestin | Hs00707120_s1 |
| MICALL1 | MICAL-like 1 | Hs00411017_m1 |
| WBP2NL | WBP2 N-terminal like | Hs00379258_m1 |
| TRIM48 | tripartite motif-containing 48 | Hs02520296_g1 |
| SH3BGRL3 | SH3 domain binding glutamic acid-rich protein like 3 | Hs00606773_g1 |
| LDLR | low density lipoprotein receptor | Hs00181192_m1 |
| RAB7L1 | RAB7, member RAS oncogene family-like 1 | Hs00187510_m1 |
| WEE1 | WEE1 homolog (S. pombe) | Hs00268721_m1 |

### F. Example 6

### Classification of IBS and Normal Status Using Patterns of Expression in Peripheral Blood

The results found in Example 5 were then applied to determine the ability of minimal gene sets to classify IBS verse normal status using expression patterns in peripheral blood. All data analysis was performed using R version 2.7.2. Raw data was log transformed to achieve a distribution closer to Gaussian distribution. The 0 value was replaced by 50% of the minimum of detected values for each gene. After removing one sample (#3) due to missing values, a 62-by-28 data matrix was formed. IBS patient samples were combined together as label “1” and healthy volunteers were labeled of “0”. A standard t-test was performed between disease and healthy stages for each gene and the p-value and difference between means are listed in Table 7. Genes were selected based on a combined criteria of p.value <0.01 and abs (difference.mean)>0.5. Prediction was performed in R using 4 different machine learning algorithms were tested to build a model to predict disease stage from healthy stage. Table 9 shows the accuracy of prediction of IBS when different models were established.

TABLE 9
Prediction of IBS by gene expression analysis
using four different prediction models.

| Prediction Models | All genes | Selected genes |
|---|---|---|
| Shrunken Centroid (PAM) | 79% | |
| Random Forest | 80% | 82% |
| Support Vector Machine Model | 74% | 82% |
| Neural Network model | 71% | 77% |

### Shrunken Centroid (PAM) Model

The Shrunken Centroid (PAM) model was built based on shrunken centroid algorithm implemented in the “pamr” package in R. The model is consisted of 24 genes and the leave-one-out accuracy was 79%.

### Random Forest Model

The second prediction model we used was based on the random forest algorithm implemented in the “randomForest” package in R. The first model was based on the entire gene set and the second model was based on the 7 genes selected from the t-test. The leave-one-out accuracies of the two models are 80% and 82% respectively.

### Support Vector Machine Model:

Two models were built based on the support vector machine algorithm implemented in the “svm” package in R. The first model was based on the entire gene set and the second model was based on the 7 genes selected from the t-test. The leave-one-out accuracies of the two models are 74% and 82% respectively.

### Neural Network Model:

Two models were built based on the neural network algorithm implemented in the “nnet” package in R. The first model was based on the entire gene set and the second model was based on the 7 genes selected from the t-test. The leave-one-out accuracies of the two models are 71% and 77% respectively.

As shown in Table 10, gene ontology analysis of the 66 DEGs identified in Example 5 reveal that 6 gene ontologies are significantly associated with the DEGs between IBS-D and healthy volunteers, including ribosome, protein biosynthesis, RNA binding, intracellular, signal transduction, and protein binding ontologies. The biological functions of 7 particularly useful genes for the diagnosis and prognosis of IBS are outlined in Table 11.

TABLE 10
Gene ontology enrichment analysis of 66 DEGs. 6 gene ontologies are
significantly associated with the DEGs between IBS-D and healthy volunteers.

| DB_Term | DB_category | p_value | odds_ratio | 95% CI |
|---|---|---|---|---|
| ribosome | cellular_component | 2.70E−07 | 49.52 | 13.31-156.96 |
| protein biosynthesis | biological_process | 2.00E−06 | 32.69 | 8.83-103.07 |
| RNA binding | molecular_function | 5.40E−06 | 18.88 | 5.62-57.69 |
| intracellular | cellular_component | 0.0062 | 4.69 | 1.40-14.27 |
| signal transduction | biological_process | 0.02 | 3.92 | 1.07-12.25 |
| protein binding | molecular_function | 0.021 | 3.33 | 1.10-10.52 |

TABLE 11
Biological functions of selected genes and their biological relevance to IBS.

| Gene | Function | Biological role |
|---|---|---|
| TACR2 (NK2) | receptor for the tachykinin | Mediate pain response, an |
| | neuropeptide substance K (neurokinin | antagonist of this receptor is |
| | A). It is associated with G proteins | under development for treating |
| | that activate a phosphatidylinositol- | IBS, which is in phase II clinical |
| | calcium second messenger system. | trial |
| VIPR1 | receptor for VIP, the activity is | VIP is a gut hormone which has |
| | mediated by G proteins which activate | been reported to be associated |
| | adenylyl cyclase | with IBS |
| MICALL1 | a cytoskeletal regulator, binds to Rab | It participates in the assembly and |
| | 13 | the activity of tight junctions. |
| Rab7L1 | GTP binding protein with GTPase | |
| | activity, involved in protein binding | |
| SH3BGRL | Belongs to the SH3BGR family, binds | |
| | to SH3 domain and has SH3/SH2 | |
| | adaptor activity | |
| GPC 2 | Cell surface proteoglycan that bears | |
| | heparan sulfate, belongs to the | |
| | glypican family | |
| CCDC147 | unknown | unclear |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for diagnosing Irritable Bowel Syndrome (IBS) in a subject in need thereof, the method comprising:
(a) isolating and/or amplifying RNA from a biological sample taken from the subject;
(b) contacting the isolated and/or amplified RNA with a detection reagent under conditions suitable to transform the detection reagent into a complex comprising the detection reagent and an IBS RNA biomarker;
(c) detecting the level of the complex; and
(d) determining if the level of the complex more closely resembles a first reference level associated with IBS or a second reference level associated with an absence of IBS, thereby diagnosing IBS in the subject,
wherein the biomarker is an RNA from a gene selected from the group consisting of those found in Table 4 such as CCDC147.

2. The method of claim 1, wherein said method comprises detecting the level of at least two IBS biomarkers selected from the group consisting of those found in Table 4 such as CCDC147 and VIPR1.

3. The method of claim 2, wherein said method comprises detecting the level of at least five IBS biomarkers selected from the group consisting of those found in Table 4.

4. The method of claim 3, wherein the biomarkers are CCDC147, VIPR1, LPAR5, CCDC144A, and GNG3.

5. The method of claim 1, wherein the biomarkers are selected from those found in Table 1.

6. The method of claim 1, wherein the biomarker is selected from the group consisting of CCDC147, VIPR1, LPAR5, CCDC144A, GNG3, ACSS2, ZNF33B, PMS2L2, RUSC1, ARHGE, ASIP, OR2L8, PI4K2A, and FOXD3.

7-11. (canceled)

12. The method of claim 1, wherein the biomarker is a mRNA molecule encoding a protein having an amino acid sequence of any one of SEQ ID NOS:1 to 75 and 154 to 162.

13. The method of claim 1, wherein the biomarker is an RNA molecule comprising a nucleic acid sequence of any one of SEQ ID NOS:76 to 162.

14. The method of claim 1, wherein said detection reagent comprises an oligonucleotide.

15. The method of claim 14, wherein the step of detecting the level of the complex comprises oligonucleotide hybridization.

16. The method of claim 15, wherein the method comprises microarray or bead-based hybridization.

17. The method of claim 14, wherein the step of detecting the level of the complex comprises nucleic acid amplification.

18. The method of claim 17, wherein the method comprises qPCR or mass spectrometry.

19. The method of claim 2, wherein the step of detecting the level of the complexes comprises quantitating the levels of a plurality of biomarkers, thereby determining a biomarker profile.

20. The method of claim 19, wherein the step of determining if the level of the complex more closely resembles a first or second reference level comprises the use of an algorithm to determine if the biomarker profile more closely resembles a first reference profile associated with IBS or a second reference profile associated with the absence of IBS.

21. (canceled)

22. (canceled)

23. (canceled)

24. (canceled)

25. The method of claim 1, wherein the biological sample is selected from the group consisting of serum, plasma, whole blood, and stool.

26. The method of claim 1, wherein the method further comprises the detection of a biomarker selected from the group consisting of a cytokine, a growth factor, an anti-neutrophil antibody, an anti-Saccharomyces cerevisiae antibody (ASCA), an antimicrobial antibody, mast cell marker, stress marker, gastrointestinal hormone, serotonin metabolite, serotonin pathway marker, carbohydrate deficient transferrin (CDT), lactoferrin, an anti-tissue transglutaminase (tTG) antibody, a lipocalin, a matrix metalloproteinase (MMP), a complex of lipocalin and MMP, a tissue inhibitor of metalloproteinases (TIMPs), a globulin (e.g., an alpha-globulin), an actin-severing protein, an S100 protein, a fibrinopeptide, calcitonin gene-related peptide (CGRP), a tachykinin, ghrelin, neurotensin, corticotropin-releasing hormone (CRH), elastase, C-reactive protein (CRP), lactoferrin, an anti-lactoferrin antibody, calprotectin, hemoglobin, NOD2/CARD15, serotonin reuptake transporter (SERT), tryptophan hydroxylase-1, 5-hydroxytryptamine (5-HT), lactulose, serine protease, prostaglandin, histamine, and a combination thereof.

27-38. (canceled)

39. The method of claim 1, wherein the method further comprises determining a symptom profile, wherein said symptom profile is determined by identifying the presence or severity of at least one symptom in said individual; and classifying said sample as an IBS sample or non-IBS sample using an algorithm based upon said diagnostic marker profile and said symptom profile.

40-47. (canceled)

48. A method for monitoring the progression or regression of Irritable Bowel Syndrome (IBS) in a subject, said method comprising:
(a) determining a first biomarker profile from a first biological sample taken from the subject at a first point in time;
(b) determining a second biomarker profile from a second biological sample taken from the subject at a second point in time; and
(c) comparing said first and said second biomarker profiles to (i) determine which biomarker profile most resembles or least resembles a first reference profile associated with IBS, (ii) determine which biomarker profile least resembles or most resembles a second reference profile associated with the absence of IBS, or (iii) determining at least 2 of the foregoing resemblances,
wherein said biomarker profiles comprise information about the expression of at least 2 biomarkers found in Table 4,
thereby monitoring progression or regression of IBS in said subject.

49. (canceled)

50. (canceled)

51. (canceled)

52. (canceled)

53. A method for assigning therapy for IBS to a subject in need thereof, the method comprising:
(a) isolating and/or amplifying RNA from a biological sample taken from the subject;
(b) contacting the isolated and/or amplified RNA with a detection reagent under conditions suitable to transform the detection reagent into a complex comprising the detection reagent and an IBS RNA biomarker;
(c) detecting the level of the complex;
(d) determining if the level of the complex more closely resembles a first reference level associated with IBS or a second reference level associated with an absence of IBS; and
(e) assigning therapy for IBS if said level more closely resembles said first reference level associated with IBS, wherein the IBS RNA biomarker is selected from the group consisting of those found in Table 4.

54-62. (canceled)
